(19) United States
(12) Patent Application Publication
(10) Pub. No.: US 2005/0065178 A1
(43) Pub. Date: Mar. 24, 2005

(54) Substituted diazabicycloakane derivatives

(76) Inventors: Anwer Basha, Lake Forest, IL (US); William H. Bunnelle, Mundelein, IL (US); Michael J. Dart, Highland Park, IL (US); Megan E. Gallagher, Chicago, IL (US); Jianguo Ji, Libertyville, IL (US); Tao Li, Grayslake, IL (US); Jennifer M. Pace, Grayslake, IL (US); Keith B. Ryther, Round Lake Park, IL (US); Karin R. Tietje, Mundelein, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/666,884

(22) Filed: Sep. 19, 2003

(51) Int. Cl. C07D 471/02; A61K 31/4745

(52) U.S. Cl. 514/300; 546/113

(57) ABSTRACT

Compounds of formula (I)
Z-Ar₁—Ar₂ (I)
wherein Z is a diazabicyclic amine, Ar₁ is a 5- or 6-membered aromatic ring, and Ar₂ is selected from an unsubstituted or substituted 5-membered heteroaryl ring; an unsubstituted or substituted 6-membered heteroaryl ring; 3,4-(methylenedioxy)phenyl; and phenyl substituted with 0, 1, 2, or 3 substituents in the meta- or para-positions. The compounds are useful in treating conditions or disorders prevented by or ameliorated by α7 nAChR ligands. Also disclosed are pharmaceutical compositions comprising compounds of formula (I) and methods for using such compounds and compositions.

### BACKGROUND OF THE INVENTION

1. Technical Field

The invention relates to diazabicycloalkane derivatives, compositions comprising such compounds, and methods of treating conditions and disorders using such compounds and compositions.

2. Description of Related Technology

Nicotinic acetylcholine receptors (nAChRs) are widely distributed throughout the central (CNS) and peripheral (PNS) nervous systems. Such receptors play an important role in regulating CNS function, particularly by modulating release of a wide range of neurotransmitters, including, but not necessarily limited to acetylcholine, norepinephrine, dopamine, serotonin and GABA. Consequently, nicotinic receptors mediate a very wide range of physiological effects, and have been targeted for therapeutic treatment of disorders relating to cognitive function, learning and memory, neurodegeneration, pain and inflammation, psychosis and sensory gating, mood and emotion, among others.

Many subtypes of the nAChR exist in the CNS and periphery. Each subtype has a different effect on regulating the overall physiological function. Typically, nAChRs are ion channels that are constructed from a pentameric assembly of subunit proteins. At least 12 subunit proteins, α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition (α4)₂(β2)₃ (the α4β2 subtype), while another major population of receptors is comprised of the homomeric (α7)₅ (the α7 subtype).

Certain compounds, like the plant alkaloid nicotine, interact with all subtypes of the nAChRs, accounting for the profound physiological effects of this compound. While nicotine has been demonstrated to have many beneficial properties, not all of the effects mediated by nicotine are desirable. For example, nicotine exerts gastrointestinal and cardiovascular side effects that interfere at therapeutic doses, and its addictive nature and acute toxicity are well-known. Ligands that are selective for interaction with only certain subtypes of the nAChR offer potential for achieving beneficial therapeutic effects with an improved margin for safety.

The α7 nAChRs have been shown to play a significant role in enhancing cognitive function, including aspects of leaming, memory and attention (Levin, E. D., J. Neurobiol. 53: 633-640, 2002). For example, α7 nAChRs have been linked to conditions and disorders related to attention deficit disorder, attention deficit hyperactivity disorder (ADHD), Alzheimer's disease (AD), mild cognitive impairment, senile dementia, dementia associated with Lewy bodies, dementia associated with Down's syndrome, AIDS dementia, Pick's Disease, as well as cognitive deficits associated with schizophrenia, among other systemic activities. The activity at the α7 nAChRs can be modified or regulated by the administration of α7 nAChR ligands. The ligands can exhibit antagonist, agonist, partial agonist, or inverse agonist properties. Thus, α7 ligands have potential in treatment of various cognitive disorders.

Although various classes of compounds demonstrating α7 nAChR-modulating activity exist, it would be beneficial to provide additional compounds demonstrating activity at the α7 nAChRs that can be incorporated into pharmaceutical compositions useful for therapeutic methods. Specifically, it would be beneficial to provide compounds that interact selectively with α7-containing neuronal nAChRs compared to other subtypes.

### SUMMARY OF THE INVENTION

The invention is directed to diazabicycloalkane derivative compounds as well as compositions comprising such compounds, and method of using the same. Compounds of the invention have the formula:
Z-Ar₁—Ar₂ (I)
or a pharmaceutically acceptable salt, ester, amide, or prodrug thereof, wherein:
Z is a diazabicyclic amine of the formula: 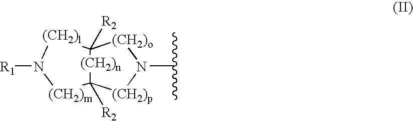
Ar₁ is a 5- or 6-membered aromatic ring of the formula: 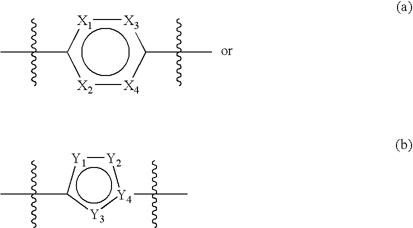
Ar₂ is selected from the group consisting of an unsubstituted or substituted 5- or 6-membered heteroaryl ring; unsubstituted or substituted bicyclic heteroaryl ring; 3,4-(methylenedioxy)phenyl; and phenyl substituted with 0, 1, 2, or 3 substituents in the meta- or para-positions; provided that when Y₁ is O or S, Y₂ is N, Y₃ is —CR₃ and R₃ is hydrogen, and Y₄ is C, then Ar₂ is not 5-tetrazolyl;
X₁, X₂, X₃, and X₄ are each independently selected from the group consisting of N and —CR₃, provided that R₃ is not hydrogen at least in one occurrence when X₁, X₂, X₃, and X₄ are all —CR₃;
Y₁, Y₂, and Y₃ are each independently selected from the group consisting of N, O, S, and —CR₃;
Y₄ is C or N, provided that when Y₄ is C at least one of Y₁, Y₂, and Y₃, is other than —CR₃;
l, m, n, o, and p are each independently selected from 0, 1, or 2, provided that the sum total of l, m, n, o, and p is 3, 4, or 5;
R₁ is independently selected from the group consisting of hydrogen, alkyl, and alkoxycarbonyl;
R₂ at each occurrence is independently selected from the group consisting of hydrogen and alkyl; and
R₃ at each occurrence is independently selected from the group consisting of hydrogen and alkyl.

Another aspect of the invention relates to pharmaceutical compositions comprising compounds of the invention. Such compositions can be administered in accordance with a method of the invention, typically as part of a therapeutic regimen for treatment or prevention of conditions and disorders related to nAChR activity, and more particularly α7 nAChR activity.

Yet another aspect of the invention relates to a method of selectively modulating to nAChR activity, for example α7 nAChR activity. The method is useful for treating and/or preventing conditions and disorders related to α7 nAChR activity modulation in mammals. More particularly, the method is useful for conditions and disorders related to attention deficit disorder, attention deficit hyperactivity disorder (ADHD), Alzheimer's disease (AD), mild cognitive impairment, senile dementia, AIDS dementia, Pick's Disease, dementia associated with Lewy bodies, and dementia associated with Down's syndrome, as well as cognitive deficits associated with schizophrenia, among other systemic activities.

The compounds, compositions comprising the compounds, and methods for treating or preventing conditions and disorders by administering the compounds are further described herein.

### DETAILED DESCRIPTION OF THE INVENTION

Definition of Terms

Certain terms as used in the specification are intended to refer to the following definitions, as detailed below.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” means an alkoxy group, as defined herein, appended to the parent molecular moiety through a carbonyl group, represented by —C(O)—, as defined herein. Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” means a straight or branched chain hydrocarbon containing from 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicylic aromatic rings. For example, bicyclic aromatic rings containing heteroatoms in a hydrocarbon ring structure are referred to as bicyclic heteroaryl rings.

The term “carbonyl” as used herein, means a —C(O)— group.

The term “carboxy” as used herein, means a —CO₂H group.

The term “cyano” as used herein, means a —CN group.

The term “formyl” as used herein, means a —C(O)H group.

The term “halo” or “halogen” means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl” means an aromatic five- or six-membered ring containing 1, 2, 3, or 4 heteroatoms independently selected from nitrogen, oxygen, or sulfur. The heteroaryl groups are connected to the parent molecular moiety through a carbon or nitrogen atom. Representative examples of heteroaryl include, but are not limited to, furyl, imidazolyl, isoxazolyl, isothiazolyl, oxadiazolyl, oxazolyl, pyrazinyl, pyrazolyl, pyridazinyl, pyridinyl, pyrimidinyl, pyrrolyl, tetrazolyl, thiadiazolyl, thiazolyl, thienyl, triazinyl, and triazolyl.

The heteroaryl groups of the invention are substituted with 0, 1, 2, or 3 substituents independently selected from alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, halo, hydroxy, hydroxyalkyl, mercapto, nitro, —NR_{A}R_{B}, (NR_{A}R_{B})alkyl, (NR_{A}R_{B})alkoxy, (NR_{A}R_{B})carbonyl, and (NR_{A}R_{B})sulfonyl.

The term “bicyclic heteroaryl” refers to fused aromatic nine- and ten-membered bicyclic rings containing 1, 2, 3, or 4 heteroatoms independently selected from nitrogen, oxygen, or sulfur, or a tautomer thereof. The bicyclic heteroaryl groups are connected to the parent molecular moiety through a carbon or nitrogen atom. Representative examples of bicyclic heteroaryl rings include, but are not limited to, indolyl, benzothiazolyl, benzofuranyl, isoquinolinyl, and quinolinyl. Bicyclic heteroaryl groups of the invention are substituted with 0, 1, 2, or 3 substituents independently selected from alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, halo, hydroxy, hydroxyalkyl, mercapto, nitro, —NR_{A}R_{B}, (NR_{A}R_{B})alkyl, (NR_{A}R_{B})alkoxy, (NR_{A}R_{B})carbonyl, and (NR_{A}R_{B})sulfonyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “—NR_{A}R_{B}” as used herein, means two groups, R_{A} and R_{B}, which are appended to the parent molecular moiety through a nitrogen atom. R_{A} and R_{B} are each independently hydrogen, alkyl, alkylcarbonyl, or formyl. Representative examples of —NR_{A}R_{B} include, but are not limited to, amino, methylamino, acetylamino, and acetylmethylamino.

The term “(NR_{A}R_{B})alkyl” as used herein, means a —NR_{A}R_{B} group, as defined herein, appended to the parent molecular moiety through an alkyl group, as defined herein. Representative examples of (NR_{A}R_{B})alkyl include, but are not limited to, (amino)methyl, (dimethylamino)methyl, and (ethylamino)methyl.

The term “(NR_{A}R_{B})alkoxy” as used herein, means a —NR_{A}R_{B} group, as defined herein, appended to the parent molecular moiety through an alkoxy group, as defined herein. Representative examples of (NR_{A}R_{B})alkoxy include, but are not limited to, (amino)methoxy, (dimethylamino)methoxy, and (diethylamino)ethoxy.

The term “(NR_{A}R_{B})carbonyl” as used herein, means a —NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, and (ethylmethylamino)carbonyl.

The term “(NR_{A}R_{B})sulfonyl” as used herein, means a —NR_{A}R_{B} group, as defined herein, appended to the parent molecular moiety through a sulfonyl group, as defined herein. Representative examples of (NR_{A}R_{B})sulfonyl include, but are not limited to, aminosulfonyl, (methylamino)sulfonyl, (dimethylamino)sulfonyl, and (ethylmethylamino)sulfonyl.

Although typically it may be recognized that an asterisk is used to indicate that the exact subunit composition of a receptor is uncertain, for example α3β4* indicates a receptor that contains the α3 and β4 proteins in combination with other subunits, the term α7 as used herein is intended to include receptors wherein the exact subunit composition is both certain and uncertain. For example, as used herein α7 includes homomeric (α7)₅ receptors and α7* receptors, which denote a nAChR containing at least one α7 subunit.

Compounds of the Invention

Compounds of the invention can have the formula (I) as described above. More particularly, compounds of formula (I):
Z-Ar₁—Ar₂ (I)
are those wherein Z is a moiety of the formula (II): 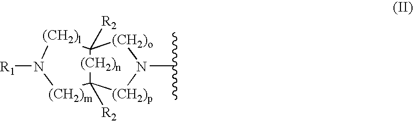
wherein R₁, R₂, Ar₁, Ar₂, l, m, n, o, and p are as previously defined. The variables l, m, n, o, and p denote numbers that are each independently selected from 0, 1, or 2, provided that the sum total of l, m, n, o, and p is 3, 4, or 5, such that the group represented by Z is a 7-, 8-, or 9-membered diazabicycloalkane, respectively. Preferably, Z is an 8-membered ring. In one particular embodiment, n is zero, such that Z is a fused bicyclic ring.
Z can have substituents represented by R₁ and R₂. Examples of moieties suitable for Z can include, but are not limited to: 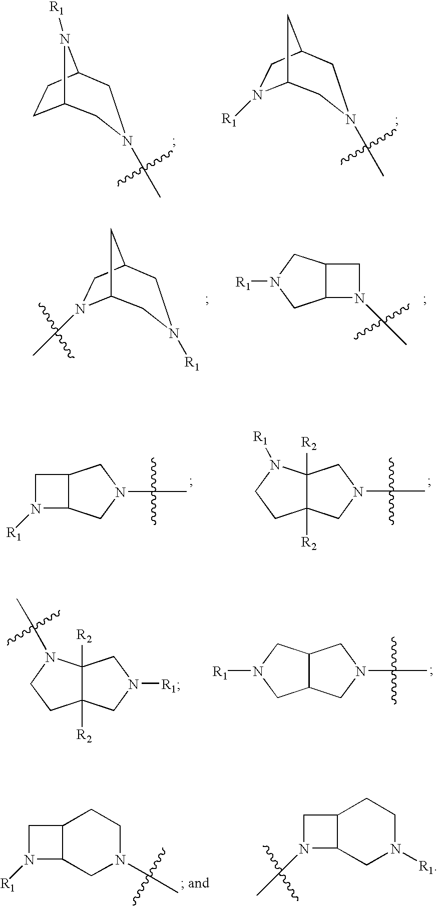
   The substituent represented by R₁ can be selected from hydrogen, alkyl, and alkoxycarbonyl. As previously described, R₂ can be selected from hydrogen and alkyl, particularly methyl.

The Ar₁ moiety can be selected independently of the moiety selected for Z. Suitable moieties for Ar₁ are those represented by a 5- or 6-membered aromatic ring of the formula:

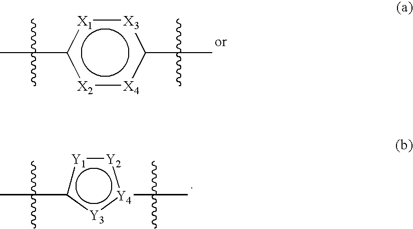

In such moieties, X₁, X₂, X₃, and X₄ are each independently selected from the group consisting of N and —CR₃, provided that R₃ is not hydrogen at least in one occurrence when X₁, X₂, X₃, and X₄ all are —CR₃, such that a phenyl group contains at least one substituent. The moiety is attached to the diazabicyclic amine and the Ar₂ moiety by 1,4-substitution or para-attachment. Preferably, the moiety represented by formula (a) contains at least one heteroatom, particularly when Ar₂ is a phenyl group.

Formula (b) represents a five-membered ring wherein Y₁, Y₂, and Y₃ are each independently selected from the group consisting of N, O, S, and —CR₃. Y₄ is selected from C or N. When Y₄ is C at least one of the substituents represented by Y₁, Y₂, and Y₃, is other than —CR₃, such that the moiety represented by formula (b) contains at least one heteroatom. The moiety generally is attached to the diazabicyclic amine and the Ar₂ moiety by 1,3-substitution.

Examples of specific rings suitable for Ar₁ include, but are not limited to, isoxazolyl, oxadiazolyl, pyrazolyl, pyridazinyl, pyridinyl, pyrimidinyl, thiadiazolyl, thiazolyl, thienyl, and phenyl substituted with 0 or 1 alkoxy substitutent. More particularly, the rings represented by Ar₁ are, for example, 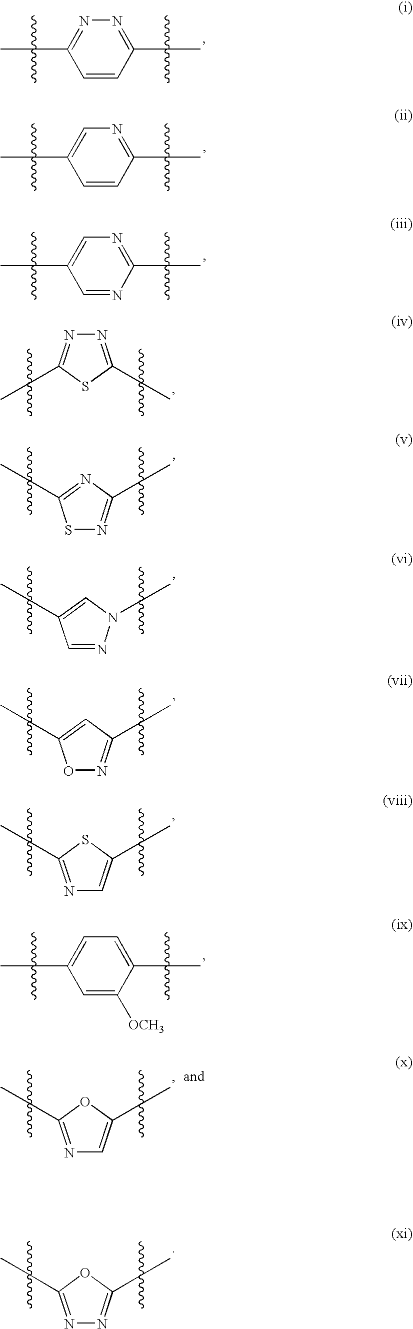
The preferred ring is pyridazinyl, for example as identified by (i). Other preferred rings for Ar₁ also are pyridinyl, for example as identified by (ii); thiadiazolyl, for example as identified by (iv); isoxazolyl, for example as identified by (vii); thiazolyl, for example as identified by (viii); oxazolyl, for example as identified by (x); and oxadiazolyl, for example as identified by (xi).

Ar₂ generally can be independently selected regardless of the moiety selected for Z or Ar₁. When Ar₂ is phenyl or substituted phenyl, Ar₁ preferably contains at least one heteroatom. Moieties suitable for Ar₂ can be an unsubstituted or substituted 5- or 6-membered heteroaryl ring; an unsubstituted or substituted bicyclic heteroaryl ring; 3,4-(methylenedioxy)phenyl; or phenyl substituted with 0, 1, 2, or 3 substituents in the meta- or para-position.

Examples of heteroaryl or bicyclic heteroaryl rings are, for example, furyl, imidazolyl, isoxazolyl, isothiazolyl, oxadiazolyl, oxazolyl, pyrazinyl, pyrazolyl, pyridazinyl, pyridinyl, pyrimidinyl, pyrrolyl, tetrazolyl, thiadiazolyl, thiazolyl, thienyl, triazinyl, triazolyl, indolyl, benzothiazolyl, benzofuranyl, isoquinolinyl, and quinolinyl. Suitable substituents for the heteroaryl and bicyclic heteroaryl ring include, but are not limited to, alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, halogen, hydroxy, hydroxyalkyl, mercapto, nitro, —NR_{A}R_{B}, wherein R_{A} and R_{B} are each independently selected from hydrogen, alkyl, alkylcarbonyl, or formyl, (NR_{A}R_{B})alkyl, (NR_{A}R_{B})alkoxy, (NR_{A}R_{B})carbonyl, and (NR_{A}R_{B})sulfonyl. More particularly, Ar₂ is selected from furyl, thienyl, pyridyl, and benzothienyl.

Phenyl and substituted phenyl groups, for example benzodioxolyl and 3,4-(methylenedioxy)phenyl, also are suitable for Ar₂. Additional suitable substituents for the phenyl ring can include, but are not limited to, alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, halogen, hydroxy, hydroxyalkyl, mercapto, nitro, —NR_{A}R_{B}, wherein R_{A} and R_{B} are each independently selected from hydrogen, alkyl, alkylcarbonyl, or formyl, (NR_{A}R_{B})alkyl, (NR_{A}R_{B})alkoxy, (NR_{A}R_{B})carbonyl, (NR_{A}R_{B})sulfonyl, and phenyl. For example, Ar₂ can be phenyl substituted with 0, 1, or 2 substituents, such as alkoxy, alkoxycarbonyl, alkyl, alkylcarbonyl, carboxy, halogen, haloalkyl, —NR_{A}R_{B}, (NR_{A}R_{B})alkyl, (NR_{A}R_{B})alkoxy, and phenyl. More specific examples of moieties suitable for Ar₂ include, but are not limited to: 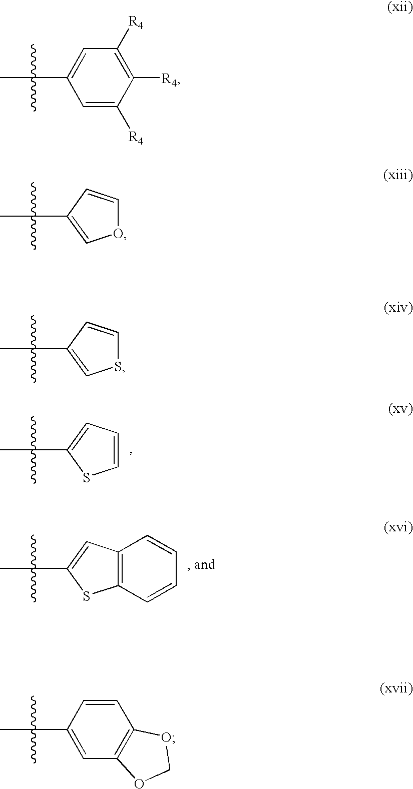
wherein R₄ at each occurrence is independently selected and represents a substituent selected from the group consisting of hydrogen, alkoxy, alkoxycarbonyl, alkyl, alkylcarbonyl, carboxy, halogen, haloalkyl, —NR_{A}R_{B}, (NR_{A}R_{B})alkyl, (NR_{A}R_{B})alkoxy, and phenyl. Preferably, the substituent represented by R₄ is selected from the group consisting of hydrogen, alkoxy, alkoxycarbonyl, alkyl, alkylcarbonyl, —NR_{A}R_{B}, and haloalkyl. Preferred moieties for Ar₂, particularly when Ar₁ is heteroaryl, are phenyl, m-methylphenyl, p-methoxyphenyl, m-trifluoromethylphenyl, m-aminophenyl, and the like. When ring of formula (b) is defined by Y₁ is O or S, Y₂ is N, Y₃ is —CR₃ and R₃ is hydrogen, and Y₄ is C, then Ar₂ is not 5-tetrazolyl.

One example of a particular embodiment of the compounds for the invention is wherein Z is a seven-membered bicyclic ring, for example 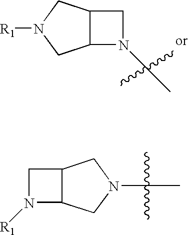
Ar₁ is pyridazinyl or pyridinyl, and Ar₂ is as described, either generally or particularly, and more particularly Ar₂ is 3,4-(methylenedioxy)phenyl, phenyl, or phenyl substituted with 0, 1, or 2 substituents selected from alkyl and alkylcarbonyl.

Another example of a particular embodiment of the compounds for the invention is wherein Z is an eight-membered bicyclic ring, for example 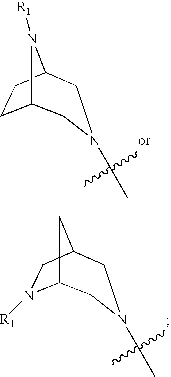
Ar₁ is pyridazinyl, and Ar₂ is as described, either generally or particularly, and more particularly Ar₂ is phenyl or phenyl substituted with a substituent selected from the group consisting of alkyl, alkoxy, haloalkyl, —NR_{A}R_{B}, and phenyl.

Yet another example of a particular embodiment of the compounds for the invention is wherein Z is 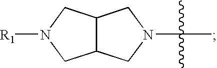
Ar₁ is pyridazinyl; and Ar₂ is as described, or more particularly, phenyl or phenyl substituted with a substituent selected from the group consisting of alkyl, alkoxy, haloalkyl, —NR_{A}R_{B}, and phenyl.

Still yet another example of a particular embodiment of the compounds for the invention is wherein Z is an eight-membered bicyclic ring, for example, 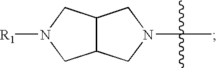
Ar₁ is pyridinyl; and Ar₂ is as described, or more particularly, furyl, benzothiophenyl, phenyl, or phenyl substituted with 0, 1, or 2 substituents selected from the group consisting of alkyl, alkoxy, haloalkyl, —NR_{A}R_{B}, and phenyl. Particularly in this embodiment, Ar₂ preferably is heteroaryl or bicyclic heteroaryl when Ar₁ is pyridinyl, provided that Ar₂ is not 1-pyrrolyl or 1-indolyl.

Yet another example of a particular embodiment of the compounds for the invention is wherein Z is an eight-membered bicyclic ring, for example, 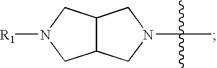
Ar₁ is either isoxazolyl, oxadiazolyl, pyrazolyl, pyrimidinyl, thiadiazolyl, or thiazolyl; and Ar₂ is as described, or more particularly, phenyl or phenyl substituted with 0, 1, or 2 substituents selected from the group consisting of alkyl, alkoxy, haloalkyl, —NR_{A}R_{B}, and phenyl.

Another example of a particular embodiment of the compounds for the invention is a nine-membered bicyclic ring, for example wherein Z is 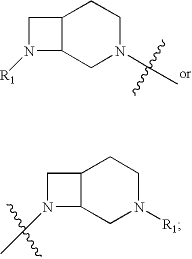
Ar₁ is pyridazinyl, pyrimidinyl, or thiazolyl; and Ar₂ is as described, or more particularly, phenyl, phenyl substituted with alkylcarbonyl, or 3,4-(methylenedioxy)phenyl.

Specific embodiments contemplated include, but are not limited to, compounds of formula (I), as defined, wherein:
3-(6-phenyl-pyridazin-3-yl)-3,8-diaza-bicyclo[3.2.1]octane;
8-methyl-3-(6-phenyl-pyridazin-3-yl)-3,8-diaza-bicyclo[3.2.1]octane;
6-methyl-3-(6-phenyl-pyridazin-3-yl)-3,6-diaza-bicyclo[3.2.1]octane;
3-(6-phenyl-pyridazin-3-yl)-3,8-diaza-bicyclo[4.2.0]octane;
8-methyl-3-(6-phenyl-pyridazin-3-yl)-3,8-diaza-bicyclo[4.2.0]octane;
2-(6-phenyl-pyridazin-3-yl)-octahydro-pyrrolo[3,4-c]pyrrole;
2-methyl-5-(6-phenyl-pyridazin-3-yl)-octahydro-pyrrolo[3,4-c]pyrrole;
2-(6-m-tolyl-pyridazin-3-yl)-octahydro-pyrrolo[3,4-c]pyrrole;
2-methyl-5-(6-m-tolyl-pyridazin-3-yl)-octahydro-pyrrolo[3,4-c]pyrrole;
2-[6-(4-methoxy-phenyl)-pyridazin-3-yl]-octahydro-pyrrolo[3,4-c]pyrrole;
2-(6-biphenyl-3-yl-pyridin-3-yl)-octahydro-pyrrolo[3,4-c]pyrrole;
2-(6-biphenyl-3-yl-pyridin-3-yl)-5-methyl-octahydro-pyrrolo[3,4-c]pyrrole;
2-[6-(3-trifluoromethyl-phenyl)-pyridin-3-yl]-octahydro-pyrrolo[3,4-c]pyrrole;
2-methyl-5-[6-(3-trifluoromethyl-phenyl)-pyridin-3-yl]-octahydro-pyrrolo[3,4-c]pyrrole;
3-[5-(hexahydro-pyrrolo[3,4-c]pyrrol-2-yl)-pyridin-2-yl]-phenylamine;
5-(6-furan-3-yl-pyridin-3-yl)-hexahydro-pyrrolo[3,4-c]pyrrole;
2-(6-furan-3-yl-pyridin-3-yl)-5-methyl-octahydro-pyrrolo[3,4-c]pyrrole;
2-(6-benzo[b]thiophen-2-yl-pyridin-3-yl)-octahydro-pyrrolo[3,4-c]pyrrole;
2-(6-benzo[b]thiophen-2-yl-pyridin-3-yl)-5-methyl-octahydro-pyrrolo[3,4-c]pyrrole;
2-(5-phenyl-pyridin-2-yl)-octahydro-pyrrolo[3,4-c]pyrrole;
2-methyl-5-(5-phenyl-pyridin-2-yl)-octahydro-pyrrolo[3,4-c]pyrrole;
2-(2-phenyl-pyrimidin-5-yl)-octahydro-pyrrolo[3,4-c]pyrrole;
2-methyl-5-(2-phenyl-pyrimidin-5-yl)-octahydro-pyrrolo[3,4-c]pyrrole;
diethyl-(2-{3-[6-(hexahydro-pyrrolo[3,4-c]pyrrol-2-yl)-pyridazin-3-yl]-phenoxy}-ethyl)-amine;
diethyl-(2-{3-[6-(5-methyl-hexahydro-pyrrolo[3,4-c]pyrrol-2-yl)-pyridazin-3-yl]-phenoxy}-ethyl)-amine;
2-(5-phenyl-[1,3,4]thiadiazol-2-yl)-octahydro-pyrrolo[3,4-c]pyrrole;
2-(3-phenyl-[1,2,4]thiadiazol-5-yl)-octahydro-pyrrolo[3,4-c]pyrrole;
2-methyl-5-(3-phenyl-[1,2,4]thiadiazol-5-yl)-octahydro-pyrrolo[3,4-c]pyrrole;
2-(1-phenyl-1H-pyrazol-4-yl)-octahydro-pyrrolo[3,4-c]pyrrole;
2-(2-methoxy-biphenyl-4-yl)-octahydro-pyrrolo[3,4-c]pyrrole;
2-(2-methoxy-biphenyl-4-yl)-5-methyl-octahydro-pyrrolo[3,4-c]pyrrole;
2-methyl-5-(3-phenyl-isoxazol-5-yl)-octahydro-pyrrolo[3,4-c]pyrrole;
(1S, 5S)-3-(6-phenyl-pyridazin-3-yl)-3,6-diaza-bicyclo[3.2.0]heptane;
(1S, 5S)-6-methyl-3-(6-phenyl-pyridazin-3-yl)-3,6-diaza-bicyclo[3.2.0]heptane;
(1R, 5S)-6-(6-phenyl-pyridazin-3-yl)-3,6-diaza-bicyclo[3.2.0]heptane;
(1R, 5S)-3-methyl-6-(6-phenyl-pyridazin-3-yl)-3,6-diaza-bicyclo[3.2.0]heptane;
(1R, 5R)-3-(6-phenyl-pyridazin-3-yl)-3,6-diaza-bicyclo[3.2.0]heptane;
(1R, 5R)-6-methyl-3-(6-phenyl-pyridazin-3-yl)-3,6-diaza-bicyclo[3.2.0]heptane;
(1R, 5R)-3-(6-benzo[1,3]dioxol-5-yl-pyridazin-3-yl)-3,6-diaza-bicyclo[3.2.0]heptane;
(1R, 5R)-3-(6-benzo[1,3]dioxol-5-yl-pyridazin-3-yl)-6-methyl-3,6-diaza-bicyclo[3.2.0]heptane;
(1R, 5R)-1-{4-[5-(3,6-diaza-bicyclo[3.2.0]hept-3-yl)-pyridin-2-yl]-phenyl}-ethanone;
(1R, 5R)-1-{4-[5-(6-methyl-3,6-diaza-bicyclo[3.2.0]hept-3-yl)-pyridin-2-yl]-phenyl}-ethanone;
6a-methyl-5-(6-m-tolyl-pyridin-3-yl)-octahydro-pyrrolo[3,4-b]pyrrole;
2-(5-phenyl-thiazol-2-yl)-octahydro-pyrrolo[3,4-c]pyrrole; and
2-methyl-5-(5-phenyl-thiazol-2-yl)-octahydro-pyrrolo[3,4-c]pyrrole;
   or pharmaceutically acceptable salts, esters, amides, and prodrugs thereof.

Compound names are assigned by using AUTONOM naming software, which is provided by MDL Information Systems GmbH (formerly known as Beilstein Informationssysteme) of Frankfurt, Germany, and is part of the CHEMDRAW® ULTRA v. 6.0.2 software suite.

Compounds of the invention may exist as stereoisomers wherein, asymmetric or chiral centers are present. These stereoisomers are “R” or “S” depending on the configuration of substituents around the chiral element. The terms “R” and “S” used herein are configurations as defined in IUPAC 1974 Recommendations for Section E, Fundamental Stereochemistry, Pure Appl. Chem., 1976, 45: 13-30. The invention contemplates various stereoisomers and mixtures thereof and are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Methods for Preparing Compounds of the Invention

As used in the descriptions of the schemes and the examples, certain abbreviations are intended to have the following meanings: Ac for acetyl; Bu for n-butyl; BINAP for 2,2′-bis(diphenylphosphino)-1,1′-binaphthyl; DBU for 1,8-diazabicyclo[5.4.0]undec-7-ene; DMSO for dimethylsulfoxide; EtOAc for ethyl acetate; EtOH for ethanol; Et₃N for triethylamine; Et₂O for diethyl ether; HPLC for high pressure liquid chromatography; i-Pr for isopropyl; MeOH for methanol; NBS for N-bromosuccinimide; OAc for acetate; Ph for phenyl; t-Bu for tert-butyl; and THF for tetrahydrofuran.

The reactions exemplified in the schemes are performed in a solvent appropriate to the reagents and materials employed and suitable for the transformations being effected. The described transformations may require modifying the order of the synthetic steps or selecting one particular process scheme over another in order to obtain a desired compound of the invention, depending on the functionality present on the molecule.

Nitrogen protecting groups can be used for protecting amine groups present in the described compounds. Such methods, and some suitable nitrogen protecting groups, are described in Greene and Wuts (Protective Groups In Organic Synthesis, Wiley and Sons, 1999). For example, suitable nitrogen protecting groups include, but are not limited to, tert-butoxycarbonyl (BOC), benzyloxycarbonyl (CBZ), benzyl (Bn), acetyl, and trifluoracetyl. More particularly, the BOC protecting group may be removed by treatment with an acid such as trifluoroacetic acid or hydrochloric acid. The CBZ and Bn protecting groups may be removed by catalytic hydrogenation. The acetyl and trifluoracetyl protecting groups may be removed by a hydroxide ion.

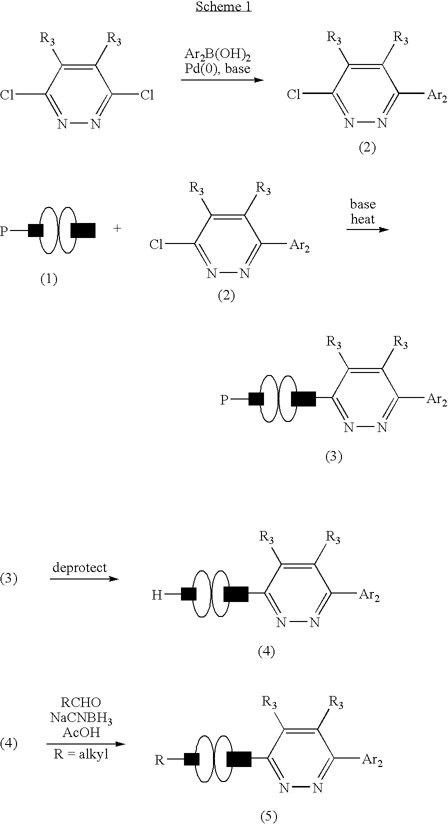

Pyridazines of general formula (4) and (5), wherein Ar₂ and R₃ are as defined in formula (I), can be prepared as described in Scheme 1. 3,6-Dichloropyridazines can be treated with a boronic acid, palladium (0), and a base to provide monochloropyridazines of general formula (2). Monochloropyridazines of general formula (2) can be treated with diazabicycles of the present invention and a base to provide pyridazines of general formula (3), wherein P is a nitrogen protecting group. Pyridazines of general formula (3) can be deprotected to provide pyridazines of general formula (4). Pyridazines of general formula (4) can be alkylated using reductive amination methods well-known to those of skill in the art to provide pyridazines of general formula (5) wherein R is alkyl.

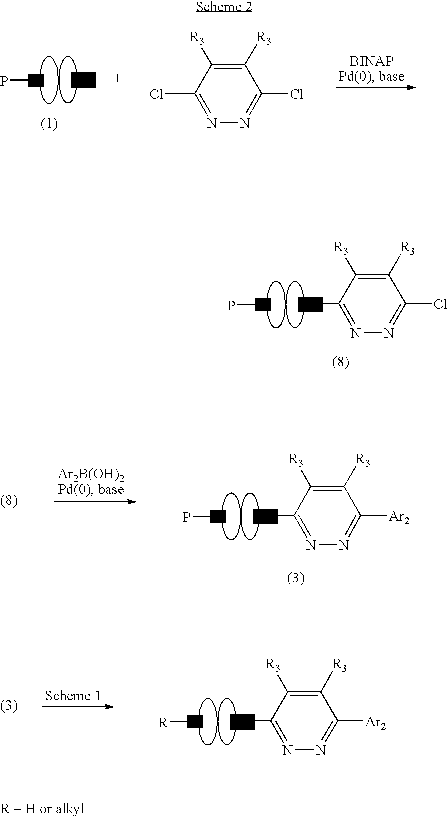

An alternative procedure for preparing pyridazines of general formula (4) and (5), wherein Ar₂ and R₃ are as defined in formula (I), is exemplified in Scheme 2. 3,6-Dichloropyridazines can be treated with diazabicycles of the present invention, palladium (0), BINAP, and a base to provide pyridazines of general formula (8), wherein P is a nitrogen protecting group. Pyridazines of general formula (8) can be treated with a boronic acid, palladium (0), and a base to provide pyridazines of general formula (3). Pyridazines of general formula (3) can be processed as described in Scheme 1 to provide pyridazines of general formula (4) and (5).

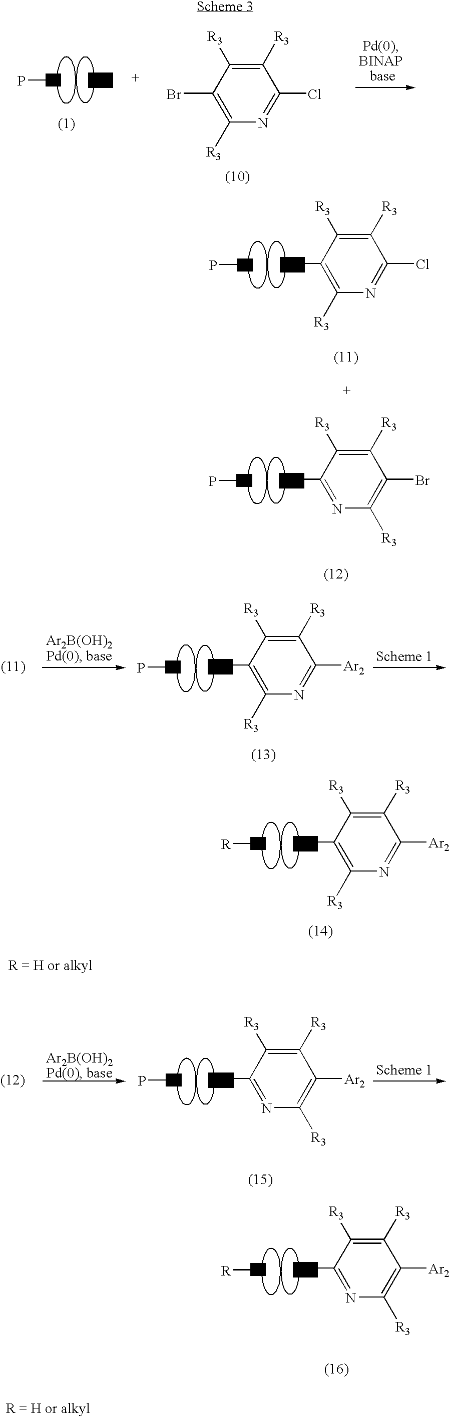

Pyridines of general formula (14) and (16), wherein Ar₂ and R₃ are as defined in formula (I), can be prepared as described in Scheme 3. 2,5-Dihalopyridines can be treated with palladium (0), BINAP, a base, and diazabicycles of the present invention wherein P is a nitrogen protecting group to provide 5-diazabicyclo-2-halopyridines of general formula (11) and 2-diazabicyclo-5-halopyridines of general formula (12). 5-Diazabicyclo-2-halopyridines of general formula (11) and 2-diazabicyclo-5-halopyridines of general formula (12) can be processed as described in Scheme 1 to provide pyridines of general formula (14) and (16).

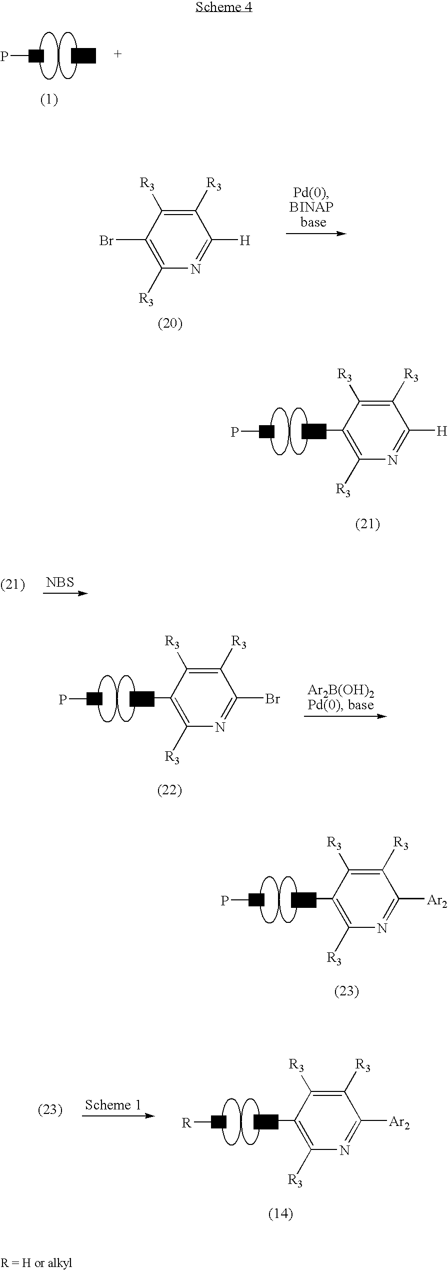

An alternative procedure for preparing pyridines of general formula (14), wherein Ar₂ and R₃ are as defined in formula (I), is exemplified in Scheme 4. Diazabicycles of the present invention, wherein P is a nitrogen protecting group, can be treated with 5-bromopyridine, BINAP, palladium (0), and a base to provide pyridines of general formula (21). Pyridines of general formula (21) can be treated with N-bromosuccinimide to provide bromides of general formula (22). Bromides of general formula (22) can be treated with a boronic acid, palladium (0), and a base to provide biarylcompounds of general formula (23). Biarylcompounds of general formula (23) can be processed as described in Scheme 1 to provide pyridines of general formula (14).

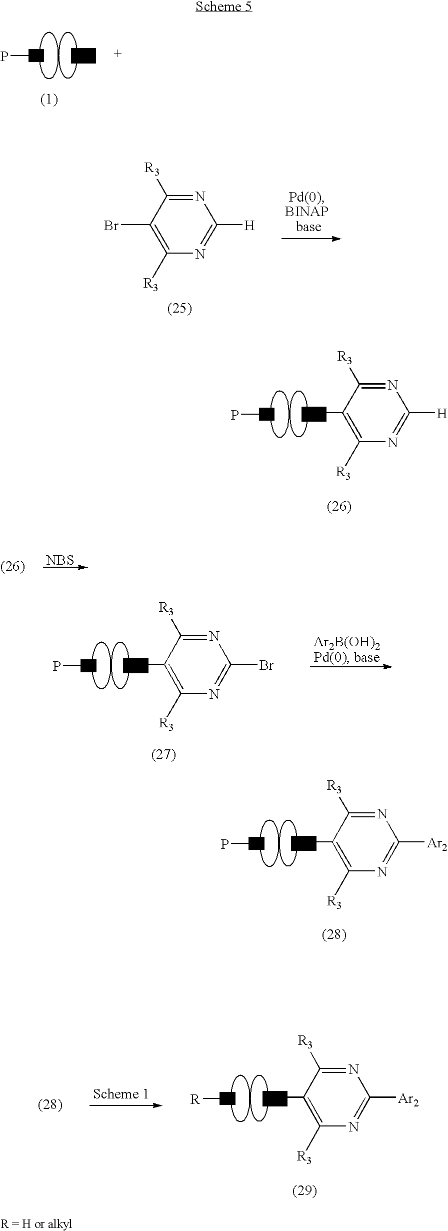

Pyrimidines of general formula (29), wherein Ar₂ and R₃ are as defined in formula (1), can be prepared as described in Scheme 5. Diazabicycles of the present invention, wherein P is a nitrogen protecting group, can be treated with 5-bromopyrimidines of general formula (25), BINAP, palladium (0), and a base to provide pyrimidines of general formula (26). Pyrimidines of general formula (26) can be treated with N-bromosuccinimide to provide bromides of general formula (27). Bromides of general formula (27) can be treated with a boronic acid, palladium (0), and a base to provide biarylcompounds of general formula (28). Biarylcompounds of general formula (28) can be processed as described in Scheme 1 to provide pyrimidines of general formula (29).

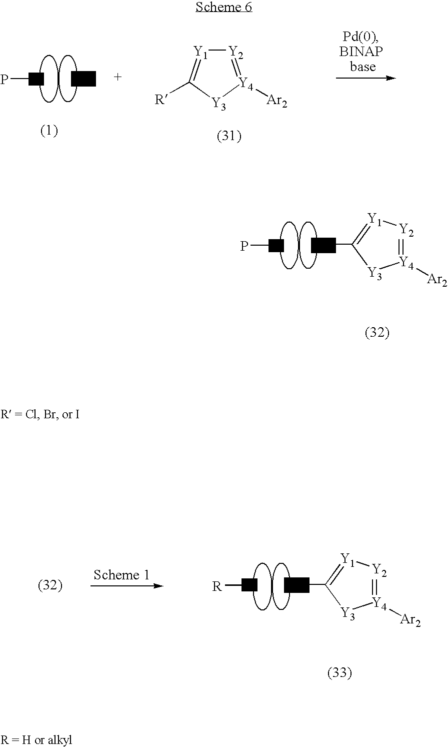

Compounds of general formula (33), wherein Ar₂, Y₁, Y₂, Y₃, and Y₄ are as defined in formula (I) can be prepared as described in Scheme 6. Diazabicyclic compounds of general formula (1), can be treated with 5-membered aromatic heteroaryls of general formula (31), purchased commercially or prepared using methodology well-known to those in the art, preferably in the presence of palladium (0), BINAP, and a base to provide compounds of general formula (32). Compounds of general formula (32) can be processed as described in Scheme 1 to provide compounds of general formula (33).

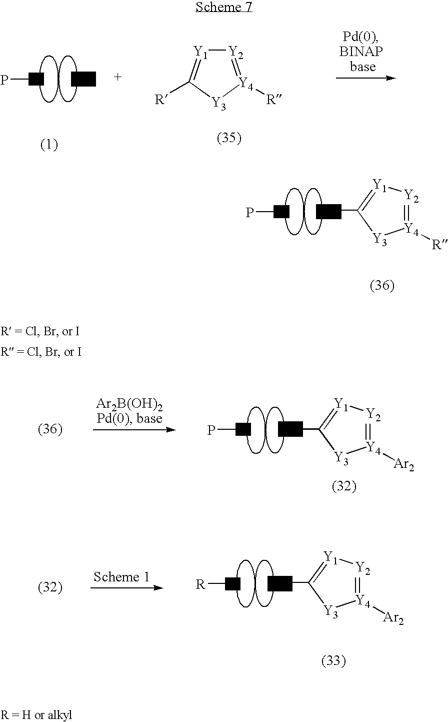

An alternate method of preparing compounds of general formula (33), wherein Ar₂, Y₁, Y₂, Y₃, and Y₄ are as defined in formula (I), is described in Scheme 7. Diazabicyclic compounds of general formula (1) can be treated with dihalo-5-membered aromatic heteroaryls of general formula (35), purchased commercially or prepared using methodology well-known to those in the art, in the presence of palladium (0), BINAP, and a base to provide monohalo compounds of general formula (36). Monohalo compounds of general formula (36) can be treated with boronic acids, palladium (0), and a base to provide compounds of general formula (32). Compounds of general formula (32) can be processed as described in Scheme 1 to provide compounds of general formula (33).

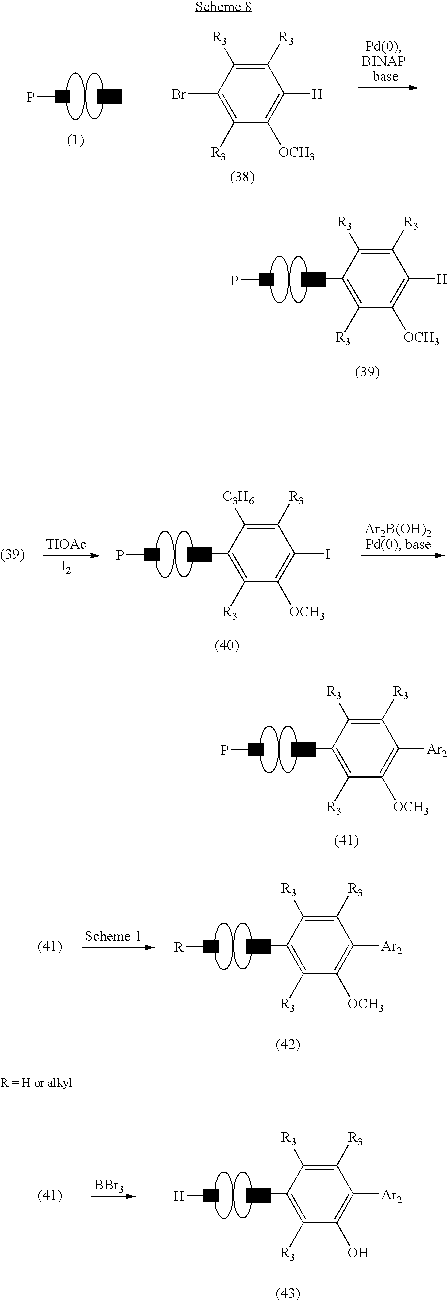

Compounds of general formula (42) and (43), wherein Ar₂ and R₃ are as defined in formula (I), can be prepared as described in Scheme 8. Diazabicycles of the present invention, wherein P is a nitrogen protecting group, can be treated with bromides of general formula (38), BINAP, palladium (0), and a base to provide compounds of general formula (39). Compounds of general formula (39) can be treated with iodine and thallium acetate to provide iodo compounds of general formula (40). Iodo compounds of general formula (40) can be treated with a boronic acid, palladium (0), and a base to provide biarylcompounds of general formula (41). Biarylcompounds of general formula (41) can be processed as described in Scheme 1 to provide compounds of general formula (42) and (43).

The compounds and intermediates of the invention may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

The compounds of the invention have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, carbonic, fumaric, gluconic, acetic, propionic, salicylic, hydrochloric, hydrobromic, phosphoric, sulfuric, citric, or hydroxybutyric acid, camphorsulfonic, malic, phenylacetic, aspartic, glutamic, and the like.

Compositions of the Invention

The invention also provides pharmaceutical compositions comprising a therapeutically effective amount of a compound of formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The compounds of the invention can be used in the form of pharmaceutically acceptable salts, esters, or amides derived from inorganic or organic acids. The term “pharmaceutically acceptable salts, esters and amides,” as used herein, include salts, zwitterions, esters and amides of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term “pharmaceutically acceptable ester,” as used herein, refers to esters of compounds of the invention which hydrolyze in vivo and include those that break down readily in the human body to leave the parent compound or a salt thereof. Examples of pharmaceutically acceptable, non-toxic esters of the invention include C₁-to-C₆ alkyl esters and C₅-to-C₇ cycloalkyl esters, although C₁-to-C₄ alkyl esters are preferred. Esters of the compounds of formula (I) can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl trifilate, for example with methyl iodide, benzyl iodide, cyclopentyl iodide. They also can be prepared by reaction of the compound with an acid such as hydrochloric acid and an alkylcarboxylic acid such as acetic acid, or with acid and an arylcarboxylic acid such as benzoic acid.

The term “pharmaceutically acceptable amide,” as used herein, refers to non-toxic amides of the invention derived from ammonia, primary C₁-to-C₆ alkyl amines and secondary C₁-to-C₆ dialkyl amines. In the case of secondary amines, the amine can also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I) can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also can be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds of formula (I).

Methods of the Invention

Compounds and compositions of the invention are useful for modulating the effects of nAChRs, and more particularly α7 nAChRs. In particular, the compounds and compositions of the invention can be used for treating and preventing disorders modulated by α7 nAChRs. Typically, such disorders can be ameliorated by selectively modulating the α7 nAChRs in a mammal, preferably by administering a compound or composition of the invention, either alone or in combination with another active agent, for example, as part of a therapeutic regimen.

The compounds of the invention, including but not limited to those specified in the examples, possess an affinity for nAChRs, and more particularly α7 nAChRs. As α7 nAChRs ligands, the compounds of the invention can be useful for the treatment and prevention of a number of α7 nAChR-mediated diseases or conditions.

For example, α7 nAChRs have been shown to play a significant role in enhancing cognitive function, including aspects of learning, memory and attention (Levin, E. D., J. Neurobiol. 53: 633-640, 2002). As such, α7 ligands are suitable for the treatment of cognitive disorders including, for example, attention deficit disorder, attention deficit hyperactivity disorder (ADHD), Alzheimer's disease (AD), mild cognitive impairment, senile dementia, AIDS dementia, Pick's Disease, dementia associated with Lewy bodies, and dementia associated with Down's syndrome, as well as cognitive deficits associated with schizophrenia.

In addition, α7-containing nAChRs have been shown to be involved in the neuroprotective effects of nicotine both in vitro (Jonnala, R. B. and Buccafusco, J. J., J. Neurosci. Res. 66: 565-572, 2001) and in vivo (Shimohama, S. et al., Brain Res. 779: 359-363, 1998). More particularly, neurodegeneration underlies several progressive CNS disorders, including, but not limited to, Alzheimer's disease, Parkinson's disease, amyotrophic lateral sclerosis, Huntington's disease, dementia with Lewy bodies, as well as diminished CNS function resulting from traumatic brain injury. For example, the impaired function of α7 nAChRs by β-amyloid peptides linked to Alzheimer's disease has been implicated as a key factor in development of the cognitive deficits associated with the disease (Liu, Q.-S., Kawai, H., Berg, D. K., PNAS 98: 4734-4739, 2001). The activation of α7 nAChRs has been shown to block this neurotoxicity (Kihara, T. et al., J. Biol. Chem. 276: 13541-13546, 2001). As such, selective ligands that enhance α7 activity can counter the deficits of Alzheimers and other neurodegenerative diseases.

Schizophrenia is a complex disease that is characterized by abnormalities in perception, cognition, and emotions. Significant evidence implicates the involvement of α7 nAChRs in this disease, including a measured deficit of these receptors in post-mortem patients (Leonard, S. Eur. J. Pharmacol. 393: 237-242, 2000). Deficits in sensory processing (gating) are one of the hallmarks of schizophrenia. These deficits can be normalized by nicotinic ligands that operate at the α7 nAChR (Adler L. E. et al., Schizophrenia Bull. 24: 189-202, 1998; Stevens, K. E. et al., Psychopharmacology 136: 320-327, 1998). Thus, α7 ligands demonstrate potential in the treatment schizophrenia.

Angiogenesis, a process involved in the growth of new blood vessels, is important in beneficial systemic functions, such as wound healing, vascularization of skin grafts, and enhancement of circulation, for example, increased circulation around a vascular occlusion. Non-selective nAChR agonists like nicotine have been shown to stimulate angiogenesis (Heeschen, C. et al., Nature Medicine 7: 833-839, 2001). Improved angiogenesis has been shown to involve activation of the α7 nAChR (Heeschen, C. et al, J. Clin. Invest. 110: 527-536, 2002). Therefore, nAChR ligands that are selective for the α7 subtype offer improved potential for stimulating angiogenesis with an improved side effect profile.

A population of α7 nAChRs in the spinal cord modulate serotonergic transmission that have been associated with the pain-relieving effects of nicotinic compounds (Cordero-Erausquin, M. and Changeus, J.-P. PNAS 98:2803-2807, 2001). The α7 nAChR ligands demonstrate therapeutic potential for the treatment of pain states, including acute pain, post-surgical pain, as well as chronic pain states including inflammatory pain and neuropathic pain. Moreover, α7 nAChRs are expressed on the surface of primary macrophages that are involved in the inflammation response, and that activation of the α7 receptor inhibits release of TNF and other cytokines that trigger the inflammation response (Wang, H. et al Nature 421: 384-388, 2003). Therefore, selective α7 ligands demonstrate potential for treating conditions involving inflammation and pain.

The mammalian sperm acrosome reaction is an exocytosis process important in fertilization of the ovum by sperm. Activation of an α7 nAChR on the sperm cell has been shown to be essential for the acrosome reaction (Son, J.-H. and Meizel, S. Biol. Reproduct. 68: 1348-1353 2003). Consequently, selective α7 agents demonstrate utility for treating fertility disorders.

Compounds of the invention are particularly useful for treating and preventing a condition or disorder affecting cognition, neurodegeneration, and schizophrenia.

Cognitive impairment associated with schizophrenia often limits the ability of patients to function normally, a symptom not adequately treated by commonly available treatments, for example, treatment with an atypical antipsychotic. (Rowley, M. et al., J. Med. Chem. 44: 477-501, 2001). Such cognitive deficit has been linked to dysfunction of the nicotinic cholinergic system, in particular with decreased activity at α7 receptors. (Friedman, J. I. et al., Biol Psychiatry, 51: 349-357, 2002). Thus, activators of α7 receptors can provide useful treatment for enhancing cognitive function in schizophrenic patients who are being treated with atypical antipsychotics. Accordingly, the combination of an α7 nAChR ligand and an atypical antipsychotic would offer improved therapeutic utility. Specific examples of suitable atypical antipsychotics include, but are not limited to, clozapine, risperidone, olanzapine, quietapine, ziprasidone, zotepine, iloperidone, and the like.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal range from about 0.10 mg/kg body weight to about 1 g/kg body weight. More preferable doses can be in the range of from about 0.10 mg/kg body weight to about 100 mg/kg body weight. If desired, the effective daily dose can be divided into multiple doses for purposes of administration. Consequently, single dose compositions may contain such amounts or submultiples thereof to make up the daily dose.

The compounds and processes of the invention will be better understood by reference to the following examples and reference examples, which are intended as an illustration of and not a limitation upon the scope of the invention.

### EXAMPLE 1

### 3-(6-Phenyl-pyridazin-3-yl)-3,8-diaza-bicyclo[3.2.1]octane bis-p-toluenesulfonate

### EXAMPLE 1A

### 5-Oxo-pyrrolidine-2-carboxylic Acid Methyl Ester

To a solution of DL-pyroglutamic acid (50 g, 0.387 mol) in 157 mL CH₃OH (3.87 mol) and 100 mL toluene was added concentrated H₂SO₄ (2.5 mL). This mixture was warmed to reflux and allowed to stir for 16 h. Since starting material remained, another 4 mL concentrated H₂SO₄ was added and the mixture stirred at reflux for an additional 24 h then was cooled to ambient temperature and 20% aqueous NaOH was added until the pH of the solution was ˜6. The mixture was concentrated under reduced pressure and the residue was dissolved in CH₂Cl₂, filtered through Celite®) diatomaceous earth, concentrated and purified via Kugelrohr distillation. The resulting material was carried on directly to the next reaction.

### EXAMPLE 1B

### 1-Benzyl-5-oxo-pyrrolidine-2-carboxylic Acid Methyl Ester

To a slurry of NaH (22 g of 60% NaH in mineral oil, 0.55 mol) in 400 mL benzene was added the product of Example 1A (0.387 mol) in 100 mL benzene dropwise via addition funnel. The mixture stirred for 30 minutes after the addition was complete, then was warmed to reflux and allowed to stir for 1.5 h. The reaction was cooled to 45° C. and stirred for 16 h. A portion of benzyl bromide (45 mL, 0.38 mol) was added, the mixture was warmed to reflux and an additional amount of benzyl bromide was added (45 mL, 0.38 mol). This solution stirred for 24 h at reflux, then was cooled to ambient temperature, filtered through Celite® diatomaceous earth and the residue was washed with CH₂Cl₂. The combined filtrates were concentrated under reduced pressure and excess benzyl bromide was removed via distillation. The distillation residue was purified via column chromatography (SiO₂, 75% hexanes-EtOAc) to give 46.6 g of the title compound (0.2 mol, 52% yield). MS (DCI/NH₃) m/z 234 (M+H)⁺.

### EXAMPLE 1C

### 1-Benzyl-5-ethoxy-2-methoxycarbonyl-3,4-dihydro-2H-pyrrolium Tetrafluoroborate

The product of Example 1B (46.6 g, 0.2 mol) in 200 mL CH₂Cl₂ was added via addition funnel to a solution of Meerwein's reagent (Et₃O⁺BF₄⁻) (Aldrich, 200 mL of 1 M solution in CH₂Cl₂, 0.2 mol) at ambient temperature. The reaction mixture stirred for 18 h then was concentrated and the residue was determined to be a 1.8:1 mixture of starting material to product. This mixture was carried on to the next step without further purification.

### EXAMPLE 1D

### 1-Benzyl-5-nitromethylene-pyrrolidine-2-carboxylic Acid Methyl Ester

To the mixture obtained in Example 1C (0.2 mol) in 130 mL CH₂Cl₂ at ambient temperature was added Et₃N (33.5 mL, 0.24 mol) followed by CH₃NO₂ (13 mL, 0.24 mol). The mixture stirred at ambient temperature for 8 h then was diluted with CH₂Cl₂, the layers were separated and the organic layer was washed with 20 mL 5% H₂SO₄ and 20 mL brine. The organic layer was dried over anhydrous Na₂SO₄, concentrated and purified via column chromatography (SiO₂, 50% hexanes-EtOAc) to give 10.2 g of the title compound (36.9 mmol). MS (DCI/NH₃) m/z 277 (M+H)⁺.

### EXAMPLE 1E

### 8-Benzyl-3,8-diaza-bicyclo[3.2.1]octan-2-one

The product of Example 1D (10.2 g, 36.9 mmol) and 5% Pt/C (2 g) in 200 mL CH₃OH was shaken under a 30 psi atmosphere of H₂ at ambient temperature for 24 h. The mixture was then filtered through Celite®) diatomaceous earth, and concentrated to give 2.88 g (13.3 mmol, 36%) of the title. MS (DCI/NH₃) m/z 217 (M+H)⁺.

### EXAMPLE 1F

### 8-Benzyl-3,8-diaza-bicyclo[3.2.1]octane

The product of Example 1E (2.88 g, 13.3 mmol) in 40 mL THF was added via cannula to a mixture of LiAlH₄ (1.52 g, 39.9 mmol) in 40 mL THF at 0° C. After the addition was complete, the reaction mixture was allowed to warm to ambient temperature and stir for 2 h. The mixture was warmed to reflux and stirred for 1 h. The reaction was cooled to 0° C. then 1.5 mL H₂O, 1.5 mL 15% NaOH and 4.5 mL H₂O were added sequentially to quench the reaction. The material was filtered, the residue was washed with EtOAc, and the filtrate was concentrated under reduced pressure and carried on directly to the next reaction. MS (DCI/NH₃) m/z 203 (M+H)⁺.

### EXAMPLE 1G

### 1-(8-Benzyl-3,8-diaza-bicyclo[3.2.1]oct-3-yl)-2,2,2-trifluoro-ethanone

To the product of Example 1F (2.0 g, 9.8 mmol) in 50 mL CH₂Cl₂ was added Et₃N (7.0 mL, 50 mmol). The mixture was cooled to 0° C. and trifluoroacetic anhydride (3.53 mL, 25 mmol) was added. The ice-bath was removed after the addition was complete and the reaction stirred for 16 h at ambient temperature. The mixture was concentrated under reduced pressure and purified by column chromatography (SiO₂, 50% hexanes-EtOAc) to give 2.5 g of the title compound (8.4 mmol, 86% yield). MS (DCI/NH₃) m/z 299 (M+H)⁺.

### EXAMPLE 1H

### 3-(2,2,2-Trifluoro-acetyl)-3,8-diaza-bicyclo[3.2.1]octane-8-carboxylic Acid Tert-Butyl Ester

To the product of Example 1G (2.5 g, 8.4 mmol) in 20 mL EtOAc, was added di-tert-butyl dicarbonate (2.0 g, 9.22 mmol) and Pd/C (10 wt %, 0.25 g). This mixture was placed under 1 atm. of H₂ via balloon and was allowed to stir for 48 h. The reaction mixture was filtered, concentrated under reduced pressure and purified via column chromatography (SiO₂, 50% hexanes-EtOAc) to give 2 g of the title compound (6.5 mmol, 77% yield). MS (DCI/NH₃) m/z 253 (M+H)⁺.

### EXAMPLE 1I

### 3,8-Diaza-bicyclo[3.2.1]octane-8-carboxylic Acid Tert-Butyl Ester

To the product of Example 1H (2.0 g, 6.5 mmol) in 57 mL CH₃OH and 11 mL H₂O was added 2.8 g K₂CO₃ (20.3 mmol). The mixture stirred for 16 h at ambient temperature then was filtered, concentrated under reduced pressure and purified via column chromatography (SiO₂, 50% hexanes-EtOAc) to give 1.2 g of the title compound (5.65 mmol, 87% yield). MS (DCI/NH₃) m/z 213 (M+H)⁺.

### EXAMPLE 1J

### 3-(6-Phenyl-pyridazin-3-yl)-3,8-diaza-bicyclo[3.2.1]octane-8-carboxylic Acid Tert-Butyl Ester

To the product of Example 11 (1.2 g, 5.65 mmol) in 50 mL toluene was added 3-chloro-6-phenylpyridazine (Aldrich, 1.62 g, 8.48 mmol), 1,3-bis(2,6-di-1-propylphenyl)imidazolium chloride (Strem, 0.144 g, 0.34 mmol), tris(dibenzylideneacetone)dipalladium (0) (Pd₂(dba)₃, Strem, 0.104 g, 0.113 mmol), and Cs₂CO₃ (2.03 g, 6.2 mmol). This mixture was degassed three times with N₂ backflushing then was warmed to 85° C. and stirred for 72 h. The reaction was cooled to ambient temperature, filtered, concentrated under reduced pressure and purified via column chromatography (SiO₂, 50% hexanes-EtOAc) to give 0.45 g of the title compound (1.2 mmol, 22% yield). MS (DCI/NH₃) m/z 367 (M+H)⁺.

### EXAMPLE 1K

### 3-(6-Phenyl-pyridazin-3-yl)-3,8-diaza-bicyclo[3.2.1]octane

The product of Example 1J (0.1 g, 0.27 mmol) in 6 mL CH₂Cl₂ at 0° C. was treated with 4 mL of trifluoroacetic acid (TFA). The ice-bath was removed after addition of the TFA and the mixture stirred for 2 h at ambient temperature then was concentrated under reduced pressure and purified via column chromatography (SiO₂, 1% NH₄OH:9% CH₃OH:90% CH₂Cl₂) to give the title compound which was carried on to the next step without further purification.

### EXAMPLE 1L

### 3-(6-Phenyl-pyridazin-3-yl)-3,8-diaza-bicyclo[3.2.1]octane bis-p-toluenesulfonate

To the product of Example 1K (0.27 mmol) in 5 mL of 10% CH₃OH in EtOAc was added p-toluenesulfonic acid (0.103 g, 0.54 mmol). Filtration of the resulting precipitate gave 0.14 g of the title compound (0.23 mmol, 85% yield).

¹H NMR (MeOH-d₄, 300 MHz) δ 2.15 (m, 4H), 2.35 (s, 6H), 3.53 (m, 1H), 3.58 (m, 1H), 4.32 (m, 3H), 4.37 (m, 1H), 7.22 (m, 4H), 7.64 (m, 3H), 7.68 (m, 4H), 7.93 (m, 2H), 7.97 (d, J=9.8 Hz, 1H), 8.36 (d, J=9.3 Hz, 1H); MS (DCI/NH₃) m/z 267 (M+H)⁺; Anal. Calculated for C₁₆H₁₈N₄.2C₇H₈O₃S: C, 59.00; H, 5.61; N, 9.17. Found: C, 58.83; H, 5.50; N, 8.88.

### EXAMPLE 2

### 8-Methyl-3-(6-phenyl-pyridazin-3-yl)-3,8-diaza-bicyclo[3.2.1]octane

### EXAMPLE 2A

### 8-Methyl-3-(6-phenyl-pyridazin-3-yl)-3,8-diaza-bicyclo[3.2.1]octane

The product of Example 1J (0.35 g, 0.96 mmol) was combined with 4 mL formaldehyde and 8 mL formic acid. This mixture was warmed to 100° C. for 3 h, then was cooled to ambient temperature, concentrated under reduced pressure and purified via column chromatography (SiO₂, 1% NH₄OH: 9% CH₃OH: 90% CH₂Cl₂) to give the title compound 0.19 g (0.68 mmol, 71%). MS (DCI/NH₃) m/z 281 (M+H)⁺.

### EXAMPLE 2B

### 8-Methyl-3-(6-phenyl-pyridazin-3-yl)-3,8-diaza-bicyclo[3.2.1]octane p-toluenesulfonate

To the product of Example 2A (0.19 g, 0.68 mmol) in 5 mL of 10% EtOH in EtOAc, was added 0.13 g of p-toluenesulfonic acid (0.68 mmol). The resulting precipitate was isolated via filtration resulting in 0.29 g of the title compound (0.623 mmol, 92% yield). ¹H NMR (MeOH-d₄, 300 MHz) δ 2.13 (m, 2H), 2.34 (m, 2H), 2.35 (s, 3H), 2.93 (br s, 3H), 3.23 (m, 1H), 3.42 (m, 1H), 4.15 (m, 2H), 4.41 (m, 2H), 7.22 (m, 2H), 7.40 (d, J=9.5 Hz, 1H), 7.49 (m, 3H), 7.69 (m, 2H), 7.93 (m, 2H), 7.97 (d, J=9.5 Hz, 1H); MS (DCI/NH₃) m/z 281 (M+H)⁺; Anal. Calculated for C₁₇H₂₀N₄.C₇H₈O₃S0.7H₂O: C, 61.97; H, 6.37; N, 12.04. Found: C, 62.34; H, 6.17; N, 11.68.

### EXAMPLE 3

### 6-Methyl-3-(6-phenyl-pyridazin-3-yl)-3,6-diaza-bicyclo[3.2.1]octane

### EXAMPLE 3A

### Tert-Butyl 2-Azabicyclo[2.2.1]hept-5-en-2-carboxylate

Aqueous formalin (37%, 114 mL, 1.41 mol) was added to a well-stirred solution of NH₄Cl (85.0 g, 1.59 mol) in water (250 mL). Freshly distilled cyclopentadiene (170 g, 2.58 mol) was added all at once, and the mixture was stirred vigorously at ambient temperature for 17 h. The lower, aqueous phase was separated, and was treated with di-t-butyl dicarbonate (172 g, 0.78 mol). Aqueous 1M NaOH (100 mL) was added to adjust the pH to ˜8, and the mixture was stirred for 7 h at ambient temperature with addition of solid NaOH (40 g total) to maintain pH-8. The mixture was extracted with hexanes (2×200 mL), and the combined organic phase was washed with brine (50 mL), dried over MgSO₄, and concentrated under vacuum. The residue was distilled under vacuum to provide the title compound (bp 80-92° C./10 Torr) as a pale yellow liquid that crystallized on cooling (123 g, 0.63 mol, 45% yield). ¹H NMR (CDCl₃, 300 MHz) δ 1.44 (s, 9H), 1.57 (m, 2H), 2.63 (m, 1H), 3.16 (br s, 1H), 3.31 (dd, J=9, 3 Hz, 1H), 4.55-4.73 (br m, 1H), 6.25-6.41 (br m, 2H). MS (DCI/NH₃) m/z 196 (M+H)⁺.

### EXAMPLE 3B

### 2,4-Diformyl-pyrrolidine-1-carboxylic Acid Tert-Butyl Ester

Through a solution of Example 3A (0.57 g, 2.9 mmol) in 1.5 mL acetic acid and 25 mL CH₂Cl₂ at −78° C. was bubbled 03 until the solution turned blue. O₂ was then flushed through the system for 10 min after which dimethylsulfide (0.54 mL, 7.30 mmol) was added. The mixture was slowly warmed to 20° C. and allowed to stir for 18 h. The solution was concentrated and the crude product was carried on directly to the next reaction. MS (DCI/NH₃) m/z 228 (M+H)⁺.

### EXAMPLE 3C

### 3-Benzyl-3,6-diaza-bicyclo[3.2.1]octane-6-carboxylic Acid Tert-Butyl Ester

To a solution of the crude product of Example 3B (2.92 mmol) in CH₃OH at 0° C. was added benzylamine (0.35 mL, 3.21 mmol) and NaCNBH₃ (1.83 g, 29.2 mmol). The ice-bath was removed and the mixture stirred at 20° C. for 24 h. The solution was cooled to 0° C. and 10 mL EtOAc and 10 mL H₂O were added followed by 5 mL of saturated, aqueous NaHCO₃. The layers were separated and the aqueous layer was extracted with 10 mL EtOAc. The combined organic layers were washed with 5 mL H₂O followed by 5 mL brine, then were dried over anhydrous Na₂SO₄. The mixture was filtered and the filtrate was concentrated and purified via flash column chromatography to give 0.68 g (2.25 mmol, 77% two-step yield) of the title compound. ¹H NMR (CH₃OH-d₄, 300 MHz) δ 1.37 and 1.51 (s, rotamers, 9H), 1.46 (m, 1H), 1.57 (dd, J=11.2, 7.46 Hz, 1H), 1.88 (m, 1H), 1.97 (m, 1H), 2.32 (m, 2H), 2.82 (m, 1H), 3.02 (m, 1H), 3.52 (m, 3H), 3.91 (m, 1H), 7.20 (m, 1H), 7.27 (m, 4H); MS (DCI/NH₃) m/z 303 (M+H)⁺.

### EXAMPLE 3D

### 3,6-Diaza-bicyclo[3.2.1]octane-6-carboxylic Acid Tert-Butyl Ester

To the product of Example 3C (0.553 g, 1.83 mmol) in 50 mL CH₃OH was added 111 mg Pd(OH)₂/C (20 wt %). The mixture was put under 60 psi of H₂, warmed to 50° C. and allowed to stir for 36 h. The solution was then cooled to 20° C., filtered through Celite® diatomaceous earth, and concentrated to give the desired product. ¹H NMR (CH₃OH-d₄, 300 MHz) δ 1.46 and 1.48 (s, rotamers, 9H), 1.78 (dd, J=11.2, 5.43 Hz, 1H), 1.91 (m, 1H), 2.28 (m, 1H), 2.61 (d, J=12.9 Hz, 1H), 2.82 (m, 3H), 3.41 (m, 2H), 3.93 (m, 1H); MS (DCI/NH₃) m/z 213 (M+H)⁺.

### EXAMPLE 3E

### 3-(6-Phenyl-pyridazin-3-yl)-3,6-diaza-bicyclo[3.2.1]octane-6-carboxylic Acid Tert-Butyl Ester

The product of Example 3D (0.87 g, 4.1 mmol), 3-chloro-6-phenylpyridazine (Aldrich, 0.94 g, 4.92 mmol) and triethylamine (1.7 mL, 12.3 mmol) were combined in dry toluene (30 mL) in a sealed tube and warmed to 110° C. for five days. The mixture was cooled to ambient temperature, diluted with CH₂Cl₂ (10 mL) and H₂O (10 mL) and the layers were separated. The aqueous layer was extracted with CH₂Cl₂ (2×5 mL). The combined organics were dried over Na₂SO₄, concentrated under reduced pressure, and purified via column chromatography (SiO₂, 50% hexanes-EtOAc). The title compound was isolated in low yield (0.24 g, 0.655 mmol, 16% yield). MS (DCI/NH₃) m/z 367 (M+H)⁺.

### EXAMPLE 3F

### 6-Methyl-3-(6-phenyl-pyridazin-3-yl)-3,6-diaza-bicyclo[3.2.1]octane

To the product of Example 3E (0.24 g, 0.655 mmol) in 2 mL formaldehyde was added 4 mL formic acid. This mixture was warmed to 100° C. and stirred for 2 h. The solution was concentrated and the crude product was purified via flash column chromatography (SiO₂, 1% NH₄OH:9% CH₃OH:90% CH₂Cl₂) to give the title compound which was carried on directly to the next reaction without further purification. MS (DCI/NH₃) m/z 281 (M+H)⁺.

### EXAMPLE 3G

### 6-Methyl-3-(6-phenyl-pyridazin-3-yl)-3,6-diaza-bicyclo[3.2.1]octane-p-toluenesulfonate

To the product of Example 3F (0.655 mmol) in 5 mL EtOAc was added p-toluenesulfonic acid monohydrate (0.12 g, 0.655 mmol). The resulting precipitate was isolated via filtration to give 0.18 g of the title compound (0.40 mmol, 61% two-step yield). ¹H NMR (MeOH-d₄, 300 MHz) δ 2.15 (m, 1H), 2.34 (s, 3H), 2.48 (m, 1H), 2.95 (s, 3H), 3.22 (m, 3H), 3.31 (m, 1H), 3.80 (m, 1H), 4.12 (m, 2H), 4.60 (m, 1H), 7.21 (m, 2H), 7.39 (d, J=9.5 Hz, 1H), 7.50 (m, 3H), 7.69 (m, 2H), 7.94 (m, 2H), 7.95 (d, J=9.5 Hz, 1H). MS (DCI/NH₃) m/z 281 (M+H)⁺; Anal. Calculated for C₁₇H₂₀N₄.C₇H₈O₃S: C, 63.69; H, 6.24; N, 12.038. Found: C, 63.64; H, 6.22; N, 12.25.

### EXAMPLE 4

### 3-(6-Phenyl-pyridazin-3-yl)-3,8-diaza-bicyclo[4.2.0]octane

### EXAMPLE 4A

### 3-Oxo-piperidine-1,4-dicarboxylic Acid 1-tert-butyl Ester 4-ethyl Ester

A mixture of commercially available ethyl-N-benzyl-3-oxo-4-piperidinecarboxylate hydrochloride (Aldrich, 75.4 g, 0.25 mol), di-t-butyl dicarbonate (58.5 g, 0.27 mol), Et₃N (36 mL, 0.26 mol), and Pd(OH)₂/C (7.5 g, 50% in H₂O) in 660 mL EtOH was put under 60 psi of H₂ and was shaken for 25 min. The mixture was then filtered and the filtrate was concentrated under reduced pressure to provide the title compound which was used in the next step without further purification. MS (DCI/NH₃) m/z 272 (M+H)⁺.

### EXAMPLE 4B

### 5-((1R)-1-Phenyl-ethylamino)-3,6-dihydro-2H-pyridine-1,4-dicarboxylic Acid 1-tert-butyl Ester 4-ethyl Ester

A mixture of the product of Example 4A (72 g, 0.265 mol) and D-(+)-α-methylbenzylamine (Aldrich, 35.9 mL, 0.279 mol) in 750 mL of toluene was combined in a 1 L, round-bottom flask equipped with a Dean-Stark trap. The mixture was refluxed for 36 h with water being removed via the Dean-Stark trap. After cooling to ambient temperature, the solution was concentrated and redissolved in EtOAc. Filtration through silica gel and Celite® diatomaceous earth gave the crude title compound which was carried on directly to the next reaction. MS (DCI/NH₃) m/z 375 (M+H)⁺.

### EXAMPLE 4C

### 3-((1R)-1-Phenyl-ethylamino)-piperidine-1,4-dicarboxylic Acid 1-tert-butyl Ester 4-ethyl Ester

To a mixture of the product of Example 4B (0.265 mol), NaBH(OAc)₃ (280.8 g, 1.33 mol), and 200 g of 4 Å powdered molecular sieves in 900 mL toluene in a 3-neck round bottom flask equipped with an internal thermometer, mechanical stirrer and addition funnel at 0° C. was added acetic acid (303 mL, 5.3 mol) dropwise via the addition funnel. After the addition was complete, the mixture was allowed to warm to ambient temperature and stir for 16 h. The reaction was filtered and concentrated under reduced pressure to remove as much of the acetic acid as possible. The residue was dissolved in 750 mL EtOAc and 500 mL saturated aqueous NaHCO₃ solution was added slowly to neutralize the residual acid. The layers were separated and the aqueous layer was extracted with 2×100 mL EtOAc. The combined organics were dried over Na₂SO₄ and concentrated under reduced pressure to give the title compound which was carried on to the next reaction without further purification. MS (DCI/NH₃) m/z 377 (M+H)⁺.

### EXAMPLE 4D

### 4-Hydroxymethyl-3-((1R)-1-phenyl-ethylamino)-piperidine-1-carboxylic Acid Tert-Butyl Ester

To a slurry of LiAlH₄ (0.292 mol) in 1 L tetrahydrofuran at 0° C. was added the product of Example 4C (0.265 mol) dropwise via addition funnel. The ice-bath was removed after the addition was complete and the mixture stirred at ambient temperature for 1 h. The reaction was quenched by the slow addition of approximately 100 g Na₂SO₄.10H₂O (excess). The mixture stirred for 16 h then was filtered, concentrated under reduced pressure and purified via column chromatography (SiO₂, 33% hexanes-EtOAc) to give 76.5 g of the mixture of isomers (0.23 mol, 86%). MS (DCI/NH₃) m/z 335 (M+H)⁺.

### EXAMPLE 4E

### 8-((1R)-1-Phenyl-ethyl)-3,8-diaza-bicyclo[4.2.0]octane-3-carboxylic Acid Tert-Butyl Ester

To the mixture of isomers from Example 4D (76.5 g, 0.23 mol) in 1.1 L of tetrahydrofuran at 0° C. was added Et₃N (95.8 mL, 0.687 mol) followed by methanesulfonyl chloride (23 mL, 0.30 mol). The ice-bath was removed after the additions were complete and the reaction was allowed to warm to ambient temperature and stir for 1 h. Cs₂CO₃ (excess) was added and the mixture was warmed to 60° C. and stirred for 16 h. The reaction was cooled to ambient temperature, filtered, and the filtrate was washed with 2×100 mL H₂O. The layers were separated and the aqueous layer was extracted with 2×100 mL EtOAc. The combined organics were dried over Na₂SO₄ and concentrated under reduced pressure. The material was purified and the isomers separated via column chromatography (SiO₂, 50% hexanes-EtOAc) to give 30.65 g of the major isomer (97 mmol, 42%) and 16.5 g of the minor isomer (52 mmol, 23%). MS (DCI/NH₃) m/z 317 (M+H)⁺.

### EXAMPLE 4F

### 8-((1R)-1-Phenyl-ethyl)-3,8-diaza-bicyclo[4.2.0]octane

To the minor isomer product of Example 4E (9.3 g, 29.4 mmol) in 40 mL CH₂Cl₂ at 0° C. was added 20 mL trifluoroacetic acid. The ice bath was removed after the addition and the mixture stirred at ambient temperature for 3 h then was concentrated under reduced pressure and the residue was purified via column chromatography (SiO₂, 1% NH₄OH:9% CH₃OH:90% CH₂Cl₂) to give the title compound. MS (DCI/NH₃) m/z 217 (M+H)⁺.

### EXAMPLE 4G

### 2,2,2-Trifluoro-1-[8-(1-phenyl-ethyl)-3,8-diaza-bicyclo[4.2.0]oct-3-yl]-ethanone

To the product of Example 4F (29.4 mmol) in 210 mL tetrahydrofuran (THF) at −30° C. was added triethylamine (5.15 mL, 36.8 mmol) followed by trifluoroacetic anhydride (TFAA, 4.36 mL, 30.9 mmol). The mixture was warmed to −10° C. and stirred for 30 min. The reaction was quenched with 50 mL saturated, aqueous NaHCO₃ then was diluted with 100 mL H₂O and 100 mL EtOAc. The layers were separated and the aqueous layer was extracted 2×50 mL EtOAc. The combined organic layers were dried over Na₂SO₄, filtered through silica gel and Celite® diatomaceous earth with EtOAc and the filtrate was concentrated under reduced pressure to give 8.8 g of the title compound (28.2 mmol, 96% two-step yield). MS (DCI/NH₃) m/z 313 (M+H)⁺.

### EXAMPLE 4H

### 3-(2,2,2-Trifluoro-acetyl)-3,8-diaza-bicyclo[4.2.0]octane-8-carboxylic Acid Tert-Butyl Ester

A mixture of the product of Example 4G (8.8 g, 28.2 mmol), di-t-butyl dicarbonate (6.15 g, 28.2 mmol), and 2.21 g of 20% Pd(OH)₂/C in 100 mL CH₃OH was shaken under 60 psi of H₂ for 5 h at 50° C. then for 9.5 h at ambient temperature. The reaction was filtered and concentrated under reduced pressure. ¹H-NMR indicated the presence of a bis-di-t-butyl dicarbamide-3,8-diaza-bicyclo[4.2.0]octane side product which carried on to the next step along with the crude product. MS (DCI/NH₃) m/z 326 (M+NH₄)⁺.

### EXAMPLE 4I

### 3,8-Diaza-bicyclo[4.2.0]octane-8-carboxylic Acid Tert-Butyl Ester

To the crude product of Example 4H (˜28.2 mmol) in 140 mL CH₃OH and 30 mL H₂O was added 4.7 g K₂CO₃ (33.8 mmol). The mixture stirred at ambient temperature for 16 h then was diluted with a 100 mL of a solution of 1% NH₄OH: 9% CH₃OH:90% CH₂Cl₂ and filtered through Celite® diatomaceous earth and silica gel. The filtrate was concentrated under reduced pressure and purified via column chromatography (SiO₂, 1% NH₄OH:9% CH₃OH:90% CH₂Cl₂) to give 3.3 g of the title compound (15.6 mmol, 55% yield). MS (DCI/NH₃) m/z 213 (M+H)⁺.

### EXAMPLE 4J

### 3-(6-Phenyl-pyridazin-3-yl)-3,8-diaza-bicyclo[4.2.0]octane-8-carboxylic Acid Tert-Butyl Ester

The product of Example 4I (0.28 g, 1.32 mmol), 3-chloro-6-phenylpyridazine (Aldrich, 0.28 g, 1.45 mmol), and Et₃N (0.28 mL, 1.98 mmol) were combined in 10 mL toluene in a pressure tube. The mixture was warmed to 98° C. and stirred for 46 h when thin layer chromatography indicated there was remaining starting material. An additional 0.28 mL of Et₃N (1.98 mmol) was added and the mixture stirred for an additional 24 h. The reaction mixture was cooled to ambient temperature and concentrated under reduced pressure. The residue was dissolved in 20 mL CH₂Cl₂, washed with 10 mL H₂O and 5 mL saturated NaCl, dried over Na₂SO₄ and concentrated under reduced pressure. Purification via column chromatography (SiO₂, 80% EtOAc-hexanes) gave 86 mg of the title compound (0.23 mmol, 18% yield). MS (DCI/NH₃) m/z 367 (M+H)⁺.

### EXAMPLE 4K

### 3-(6-Phenyl-pyridazin-3-yl)-3,8-diaza-bicyclo[4.2.0]octane Trifluoroacetate

The product of Example 4J (0.86 mg, 0.23 mmol) in 3 mL CH₂Cl₂ was treated with 3 mL of trifluoroacetic acid (TFA) at ambient temperature. The mixture was allowed to stir for 2 h then was concentrated under reduced pressure. Toluene (5 mL) was added and the material was again concentrated under reduced vacuum. An additional 5 mL of toluene was added and removed under reduced pressure and then the material was triturated with EtOAc and diethyl ether to give 88.8 mg of the title compound. ¹H NMR (CH₃OH-d₄, 300 MHz) δ 2.10 (dq, J=15.3, 4.1 Hz, 1H), 2.31 (m, 1H), 3.27 (m, 1H), 3.72 (ddd, J=12.2, 5.4, 3.7 Hz, 1H), 3.83 (dd, J=15.3, 3.4 Hz, 1H), 4.05 (m, 2H), 4.22 (dd, J=11.2, 9.2 Hz, 1H), 4.61 (dd, J=15.3, 3.1 Hz, 1H), 4.90 (m, 1H), 7.54 (m, 3H), 7.63 (d, J=9.8 Hz, 1H), 7.95 (m, 2H), 8.16 (d, J=9.8 Hz, 1H); MS (DCI/NH₃) m/z 267 (M+H)⁺; Anal. calculated for C₁₆H₁₈N₄.2CF₃CO₂H: C, 48.59; H, 4.08; N, 11.33. Found: C, 48.69; H, 4.34; N, 11.04.

### EXAMPLE 5

### 8-Methyl-3-(6-phenyl-pyridazin-3-yl)-3,8-diaza-bicyclo[4.2.0]octane

### EXAMPLE 5A

### 3-(6-Phenyl-pyridazin-3-yl)-3,8-diaza-bicyclo[4.2.0]octane

To the product of Example 4J (0.37 g, 1.01 mmol) in 10 mL CH₂Cl₂ was added 5 mL trifluoroacetic acid (TFA). The mixture stirred at ambient temperature for 2.5 h then was concentrated under reduced pressure. Toluene (5 mL) was added and the solution was again concentrated under reduced pressure. The residue was purified via flash column chromatography (SiO₂, 12% CH₃OH—CH₂Cl₂ with 1% NH₄OH) to give 0.24 g of the title compound (0.90 mmol, 89% yield). MS (DCI/NH₃) m/z 267 (M+H)⁺.

### EXAMPLE 5B

### 8-Methyl-3-(6-phenyl-pyridazin-3-yl)-3,8-diaza-bicyclo[4.2.0]octane

The product of Example 5A (0.24 g, 0.90 mmol), 3 mL formalin and 6 mL formic acid were combined in a sealed tube and warmed to 100° C. The reaction mixture stirred for 1 h then was cooled to ambient temperature and concentrated under reduced pressure. Purification via flash column chromatography (SiO₂, 12% CH₃OH—CH₂Cl₂ with 1% NH₄OH) gave the title compound (95 mg, 0.339 mmol, 38% yield). MS (DCI/NH₃) m/z 281 (M+H)⁺.

### EXAMPLE 5C

### 8-Methyl-3-(6-phenyl-pyridazin-3-yl)-3,8-diaza-bicyclo[4.2.0]octane p-toluenesulfonate

To the product of Example 5B (95 mg, 0.339 mmol) in 5 mL of 10% EtOH in EtOAc was added p-toluenesulfonic acid (76 mg, 0.4 mmol) in 2 mL of 10% EtOH in EtOAc. The mixture stirred at ambient temperature for 72 h then the solid was isolated via filtration to give 50 mg of the title compound (0.078 mmol, 23% yield). ¹H NMR (CH₃OH-d₄, 300 MHz) δ 2.15 (dq, J=14.9, 4.8 Hz, 1H), 2.29 (s, 6H), 2.35 (m, 1H), 3.02 (m, 3H), 3.26 (m, 1H), 3.72 (dt, J=12.9, 4.8 Hz, 1H), 3.84 (dd, J=15.3, 3.1 Hz, 1H), 4.14 (m, 2H), 4.32 (dd, J=11.2, 4.8 Hz, 1H), 4.59 (dd, J=15.6, 2.4 Hz, 1H), 4.79 (dt, J=9.5, 2.7 Hz, 1H), 7.16 (m, 4H), 7.61 (m, 3H), 7.63 (m, 4H), 7.92 (m, 2H), 7.93 (d, J=9.8 Hz, 1H), 8.28 (d, J=10.0 Hz, 1H); MS (DCI/NH₃) m/z 281 (M+H)⁺; Anal. calculated for C₁₇H₂₀N₄.2C₇H₈O₃S.H₂O: C, 57.93; H, 5.96; N, 8.72. Found: C, 57.84; H, 5.75; N, 8.62.

### EXAMPLE 6

### 2-(6-Phenyl-pyridazin-3-yl)-octahydro-pyrrolo[3,4-c]pyrrole

### EXAMPLE 6A

### 5-Benzyl-tetrahydro-pyrrolo[3,4-c]pyrrole-1,3-dione

To the maleimide (80.4 g, 0.83 mol) in 1.5 L of CH₂Cl₂ in a 3-neck, 3-L round bottom flask equipped with an addition funnel, internal thermometer, and N₂ inlet at 0° C. was added trifluoroacetic acid (TFA) (6.4 mL, 83 mmol). Benzyl(methoxymethyl)trimethylsilylmethylamine (261 g, 1.1 mol) in 500 mL CH₂Cl₂ was added dropwise via addition funnel over 3 hours with the reaction temperature being maintained below 5° C. After the addition was complete, the mixture was allowed to warm slowly to ambient temperature and then was stirred for 16 h. The mixture was concentrated and the residue was dissolved in 500 mL CH₂Cl₂ and was washed with 2×50 mL saturated NaHCO₃. The layers were separated and the aqueous layer was extracted 2×25 mL CH₂Cl₂. The combined organics were washed with 25 mL brine, dried over saturated, aqueous Na₂SO₃, and concentrated under reduced pressure to give the title compound which was carried on to the next step without further purification. MS (DCI/NH₃) m/z 231 (M+H)⁺.

### EXAMPLE 6B

### 5-Benzyl-hexahydro-pyrrolo[3,4-c]pyrrole-2-carboxylic Acid Tert-Butyl Ester

To a slurry of LiAlH₄ (25 g, 0.63 mol) in 1 L THF at 0° C. in a 3-L round bottom flask equipped with an addition funnel and an N₂ inlet, was added 48 g (0.19 mmol) of the crude product of Example 6A (0.21 mol) in 500 mL THF dropwise via the addition funnel over 3 h. After the addition was complete, the ice-bath was removed and the mixture stirred at ambient temperature for 30 min before being warmed to reflux and stirred for 4 h. The reaction was cooled to 0° C. and quenched by the slow addition of Na₂SO₄.10H₂O (excess). This mixture stirred for 16 h at ambient temperature then was filtered and the residue was washed with EtOAc. The combined filtrates were concentrated and the residue was dissolved in 500 mL THF. Di-t-butyl dicarbonate (46 g, 0.21 mol) and 100 mL saturated, aqueous NaHCO₃ were added and the mixture stirred for 16 h at ambient temperature. The reaction was quenched with 50 mL H₂O and 250 mL EtOAc was added. The layers were separated, the aqueous layer was extracted 3×50 mL EtOAc, and the combined organic layers were dried over Na₂SO₄ and concentrated under reduced pressure. Purification via column chromatography (SiO₂, 50% hexanes-EtOAc) gave 33.4 g of the title compound (0.11 mol, 53% yield). MS (DCI/NH₃) m/z 303 (M+H)⁺.

### EXAMPLE 6C

### Hexahydro-pyrrolo[3,4-c]pyrrole-2-carboxylic Acid Tert-Butyl Ester

To the product of Example 6B (107.8 g, 0.356 mol) in 250 mL CH₃OH was added 10.8 g of 20% Pd(OH)₂/C, wet. This mixture was hydrogenated for 2.5 h under 60 psi of H₂ at 50° C. The mixture was filtered and concentrated to give 74 g of the title compound (0.35 mmol, 98% yield). MS (DCI/NH₃) m/z 213 (M+H)⁺.

### EXAMPLE 6D

### 5-(6-Phenyl-pyridazin-3-yl)-hexahydro-pyrrolo[3,4-c]pyrrole-2-carboxylic Acid Tert-Butyl Ester

The product of Example 6C (0.272 g, 1.28 mmol), 3-chloro-6-phenylpyridazine (0.268 g, 1.41 mmol), and Et₃N (0.27 mL, 1.92 mmol) in 10 mL of toluene were refluxed for 48 hours. The mixture was cooled, diluted with 10 mL EtOAc, washed with 5 mL H₂O, and the layers were separated. The organic layer was dried over Na₂SO₄, concentrated under reduced pressure and purified via flash column chromatography (SiO₂, 80% EtOAc-hexanes) to give 0.16 g of the title compound (0.44 mmol, 34%). MS (DCI/NH₃) m/z 366 (M+H)⁺.

### EXAMPLE 6E

### 2-(6-Phenyl-pyridazin-3-yl)-octahydro-pyrrolo[3,4-c]pyrrole Trifluoroacetate

To a solution of the product of Example 6D (0.16 g, 0.44 mmol) in 10 mL CH₂Cl₂ was added 5 mL trifluoroacetic acid. The mixture stirred at ambient temperature for 3 h then was concentrated under reduced pressure. The crude material was azeotroped 2×5 mL toluene then 10 mL EtOAc was added and the mixture was stirred until a precipitate formed. The precipitate was isolated via filtration to give 140 mg of the title compound (0.30 mmol, 69% yield). ¹H NMR (CH₃OH-d₄, 300 MHz) δ 3.36 (m, 4H), 3.65 (m, 2H), 3.75 (dd, J=11.5, 3.1 Hz, 2H), 3.89 (m, 2H), 7.46 (d, J=9.5 Hz, 1H), 7.53 (m, 3H), 7.96 (m, 2H), 8.17 (d, J=9.8 Hz, 1H); MS (DCI/NH₃) m/z 267 (M+H)⁺; Anal. calculated for C₁₆H₁₈N₄.1.7CF₃CO₂H: C, 50.63; H, 4.31; N, 12.17. Found: C, 50.50; H, 4.14; N, 12.14.

### EXAMPLE 7

### 2-Methyl-5-(6-phenyl-pyridazin-3-yl)-octahydro-pyrrolo[3,4-c]pyrrole

### EXAMPLE 7A

### 2-(6-Phenyl-pyridazin-3-yl)-octahydro-pyrrolo[3,4-c]pyrrole

To the product of Example 6D (8.63 g, 23.5 mmol) in 50 mL CH₂Cl₂ at 0° C. was added 25 mL trifluoroacetic acid (TFA). The ice-bath was removed after the addition and the mixture stirred at ambient temperature for 4 h. Concentration under reduced pressure followed by purification via column chromatography (SiO₂, 1% NH₄OH:9% CH₃OH:90% CH₂Cl₂) gave quantitative yield of the title compound. MS (DCI/NH₃) m/z 267 (M+H)⁺.

### EXAMPLE 7B

### 2-Methyl-5-(6-phenyl-pyridazin-3-yl)-octahydro-pyrrolo[3,4-c]pyrrole

To the product of Example 7A in 25 mL 1,2-dichloroethane and 50 mL formalin was added 7.5 g NaBH(OAc)₃ (35.3 mmol). This mixture stirred at ambient temperature for 16 h then was quenched with 30 mL saturated, aqueous NaHCO₃. The layers were separated and the aqueous layer was extracted 2×15 mL CH₂Cl₂. The combined organics were dried over Na₂SO₄, concentrated under reduced pressure and purified via column chromatography (SiO₂, 1% NH₄OH:9% CH₃OH:90% CH₂Cl₂) to give 6.17 g of the title compound (22 mmol, 94% yield). MS (DCI/NH₃) m/z 281 (M+H)⁺.

### EXAMPLE 7C

### 2-Methyl-5-(6-phenyl-pyridazin-3-yl)-octahydro-pyrrolo[3,4-c]pyrrole Hydrochloride

To the product of Example 7B (6.17 g, 22 mmol) in 50 mL EtOAc was added 11 mL of 4N HCl in dioxane (44 mmol). The mixture stirred for 1 h then the precipitate was isolated via filtration to give 6.73 g of the title compound (17.7 mmol, 80% yield). ¹H NMR (CH₃OH-d₄, 300 MHz) δ 2.96 and 3.03 (rotamer s, 3H), 3.16 (m, 1H), 3.47 (m, 2H), 3.60 (m, 1H), 3.77 (m, 1H), 3.93 (m, 3H), 4.01 (m, 2H), 7.58 (m, 3H), 7.79 and 7.81 (rotamer d, J=9.4 Hz, 1H), 7.95 (m, 2H), 8.41 and 8.42 (rotamer d, J=9.4 Hz, 1H); MS (DCI/NH₃) m/z 281 (M+H)⁺; Anal. calculated for C₁₇H₂₀N₄.2HCl-1.5H₂O: C, 53.69; H, 6.63; N, 14.73. Found: C, 53.59; H, 6.72; N, 14.96.

### EXAMPLE 8

### 2-(6-m-Tolyl-pyridazin-3-yl)-octahydro-pyrrolo[3,4-c]pyrrole

### EXAMPLE 8A

### 5-(6-Chloro-pyridazin-3-yl)-hexahydro-pyrrolo[3,4-c]pyrrole-2-carboxylic Acid Tert-Butyl Ester

To the product of Example 6C (1.5 g, 7.1 mmol) in 35 mL p-dioxane was added 3,6-dichloropyridazine (Aldrich, 1.37 g, 9.2 mmol), tris(dibenzylideneacetone)dipalladium (0) (Pd₂(dba)₃, Strem, 0.28 g, 0.31 mmol), 1,3-bis(2,6-di-1-propylphenyl)imidazolium chloride (Strem, 0.38 g, 0.90 mmol), and Cs₂CO₃ (6.97 g, 21.2 mmol). This mixture was warmed to 85° C. and stirred for 18 h then was cooled to ambient temperature, filtered, and concentrated under reduced pressure. To the crude material was added 50 mL 80% EtOAc-hexanes and the resulting solids were dried under reduced pressure to give 0.81 g of the title compound (2.5 mmol, 35% yield). MS (DCI/NH₃) m/z 325 (M+H)⁺.

### EXAMPLE 8B

### 5-(6-m-Tolyl-pyridazin-3-yl)-hexahydro-pyrrolo[3,4-c]pyrrole-2-carboxylic Acid Tert-Butyl Ester

To the product of Example 8A (1.45 g, 4.47 mmol) in 50 mL p-dioxane was added m-tolylboronic acid (0.79 g, 5.82 mmol), tris(dibenzylideneacetone)dipalladium (0) (Pd₂(dba)₃, Strem, 0.24 g, 0.26 mmol), 1,3-bis(2,6-di-1-propylphenyl)imidazolium chloride (Strem, 0.312 g, 0.73 mmol), and Cs₂CO₃ (4.4 g, 13.4 mmol). This mixture was warmed to 85° C. and stirred for 20 h. The reaction was then cooled to ambient temperature and concentrated under reduced pressure. Diethyl ether and hexane were added to the crude material and the resulting solid was filtered to give 1.28 g of the title compound (3.37 mmol, 75% yield). MS (DCI/NH₃) m/z 381 (M+H)⁺.

### EXAMPLE 8C

### 2-(6-m-Tolyl-pyridazin-3-yl)-octahydro-pyrrolo[3,4-c]pyrrole Trifluoroacetate

To the product of Example 8B (0.19 g, 0.50 mmol) in 10 mL CH₂Cl₂ was added 5 mL of trifluoroacetic acid. This mixture stirred for 30 min at ambient temperature then was concentrated under reduced pressure. The crude material was azeotroped 2×5 mL toluene then was dissolved in 10% EtOH/EtOAc. Upon stirring at ambient temperature, a precipitate formed. Filtration gave 33.3 mg of the title compound (0.065 mmol, 13% yield). ¹H NMR (CH₃OH-d₄, 300 MHz) δ 2.44 (m, 3H), 3.30 (m, 1H), 3.40 (m, 3H), 3.66 (m, 2H), 3.76 (m, 2H), 3.91 (m, 2H), 7.36 (m, 1H), 7.43 (m, 1H), 7.54 (d, J=9.8 Hz, 1H), 7.70 (m, 1H), 7.79 (m, 1H), 8.22 (d, J=9.8 Hz, 1H); MS (DCI/NH₃) m/z 281 (M+H)⁺; Anal. calculated for C₁₇H₂₀N₄.2CF₃CO₂H: C, 49.61; H, 4.36; N, 11.02. Found: C, 49.88; H, 4.44; N, 11.25.

### EXAMPLE 9

### 2-Methyl-5-(6-m-tolyl-pyridazin-3-yl)-octahydro-pyrrolo[3,4-c]pyrrole Hydrochloride

The product of Example 8B (1.18 g, 3.1 mmol) in 10 mL trifluoroacetic acid (TFA) and 20 mL CH₂Cl₂ was stirred for 90 min at ambient temperature then was concentrated under reduced pressure. The crude TFA salt was azeotroped 2×15 mL toluene then was dissolved in EtOAc. Et₂O was then added dropwise with stirring until a precipitate formed. Filtration gave 1.5 g of the trifluoroacetic acid salt. This material was then dissolved in 25 mL of 37% aqueous formaldehyde and NaBH(OAc)₃ (0.82 g, 3.84 mmol) was added. The reaction stirred at ambient temperature for 18 h then was concentrated under reduced pressure. Saturated, aqueous NaHCO₃ (20 mL) was added and the layers were separated. The aqueous layer was extracted 5×10 mL CH₂Cl₂. The combined organics were washed with 1×10 mL brine then were concentrated under reduced pressure. This crude material was dissolved in 15 mL 10% EtOH-EtOAc and 4 mL of 4 N HCl was added. Et₂O was added and after stirring a precipitate formed. Filtration gave 1.0 g of the title compound (2.5 mmol, 81% yield). ¹H NMR (CH₃OH-d₄, 300 MHz) δ 2.45 (s, 3H), 2.96 and 3.02 (rotamer s, 3H), 3.20 (m, 1H), 3.48 (m, 2H), 3.61 (m, 1H), 3.79 (m, 1H), 3.98 (m, 5H), 7.44 (m, 2H), 7.81 (m, 3H), 8.41 and 8.44 (rotamer d, J=9.8 Hz, 1H); MS (DCI/NH₃) m/z 295 (M+H)⁺; Anal. calculated for C₁₈H₂₂N₄.2.5HCl.0.5H₂O: C, 54.79; H, 6.51; N, 14.20. Found: C, 54.81; H, 6.58; N, 14.24.

### EXAMPLE 10

### 2-[6-(4-Methoxy-phenyl)-pyridazin-3-yl]-octahydro-pyrrolo[3,4-c]pyrrole

### EXAMPLE 10A

### 5-[6-(4-Methoxy-phenyl)-pyridazin-3-yl]-hexahydro-pyrrolo[3,4-c]pyrrole-2-carboxylic Acid Tert-Butyl Ester

The product of Example 8A (0.5 g, 1.54 mmol), p-methoxyphenylboronic acid (Aldrich, 0.47 g, 3.1 mmol), tris(dibenzylideneacetone)dipalladium (0) (Pd₂(dba)₃, Strem, 56 mg, 0.062 mmol), 1,3-bis(2,6-di-1-propylphenyl)imidazolium chloride (Strem, 65 mg, 0.154 mmol) and 2.5 mL of 2N Na₂CO₃ were combined in 25 mL toluene. This mixture was warmed to 85° C. and stirred for 16 h. The mixture was then cooled to ambient temperature, filtered through Celite® diatomaceous earth and concentrated under reduced pressure. Purification of the crude material via column chromatography (SiO₂, 50% hexanes-EtOAc) gave 0.59 g of the title compound (1.49 mmol, 97% yield). MS (DCI/NH₃) m/z 397 (M+H)⁺.

### EXAMPLE 10B

### 2-[6-(4-Methoxy-phenyl)-pyridazin-3-yl]-octahydro-pyrrolo[3,4-c]pyrrole

To the product of Example 10A (0.10 g, 0.25 mmol) in 4 mL CH₂Cl₂ at 0° C. was added 2.5 mL trifluoroacetic acid (TFA). The ice-bath was removed after addition of the TFA and the reaction mixture stirred at ambient temperature for 2 h. Concentration under reduced pressure followed by column chromatography gave 70 mg of the title compound (0.236 mmol, 93% yield).

### EXAMPLE 10C

### 2-[6-(4-Methoxy-phenyl)-pyridazin-3-yl]-octahydro-pyrrolo[3,4-c]pyrrole Hydrochloride

To the product of Example 10B (30 mg, 0.10 mmol) in 10% EtOH-EtOAc was added 0.5 mL 4N HCl (excess). This mixture stirred at ambient temperature for 1 h then the precipitate was isolated via filtration to give 37.8 mg (0.093 mmol, 93%) of the title compound. ¹H NMR (CH₃OH-d₄, 300 MHz) δ 3.35 (m, 2H), 3.43 (m, 2H), 3.66 (m, 2H), 3.77 (m, 2H), 3.88 (s, 3H), 3.97 (m, 2H), 7.11 (m, 2H), 7.72 (d, J=9.8 Hz, 1H), 7.93 (m, 2H), 8.37 (d, J=9.8 Hz, 1H); MS (DCI/NH₃) m/z 297 (M+H)⁺; Anal. calculated for C₁₇H₂₀N₄.3HCl: C, 50.32; H, 5.71; N, 13.81. Found: C, 50.42; H, 6.11; N, 13.71.

### EXAMPLE 11

### 2-(6-Biphenyl-3-yl-pyridin-3-yl)-octahydro-pyrrolo[3,4-c]pyrrole

### EXAMPLE 11A

### 5-Pyridin-3-yl-hexahydro-pyrrolo[3,4-c]pyrrole-2-carboxylic Acid Tert-Butyl Ester

To the product of Example 6C (1.0 g, 4.7 mmol) in 15 mL toluene in a pressure tube was added 3-bromopyridine (0.54 mL, 5.65 mmol), tris(dibenzylideneacetone)dipalladium (0) (Pd₂(dba)₃, Strem, 86 mg, 0.094 mmol), racemic-2,2′-bis(diphenylphosphino)-1,1′-binaphthyl (BINAP, Strem, 0.117 g, 0.19 mmol), and tert-BuONa (0.723 g, 7.52 mmol). This mixture was warmed to 85° C. and stirred for 24 h. The reaction was then cooled to ambient temperature, filtered through Celite® diatomaceous earth, concentrated under reduced pressure and purified via column chromatography (SiO₂, 50% hexanes-EtOAc) to give the title compound (1.08 g, 3.74 mmol, 79% yield). MS (DCI/NH₃) m/z 290 (M+H)⁺.

### EXAMPLE 11B

### 5-(6-Bromo-pyridin-3-yl)-hexahydro-pyrrolo[3,4-c]pyrrole-2-carboxylic Acid Tert-Butyl Ester

To the product of Example 11A (1.0 g, 3.46 mmol) in 40 mL CH₃CN at 0° C. was added N-bromosuccinimide (0.62 g, 3.46 mmol) in 5 mL CH₃CN dropwise over 15 minutes. The mixture was stirred at 0° C. for 30 min then was allowed to warm to ambient temperature. Reaction was quenched with 10 mL H₂O and the layers were separated. The aqueous layer was extracted 2×5 mL CH₂Cl₂ and the combined organics were washed 1×5 mL brine, dried over Na₂SO₄, and concentrated under reduced pressure to give 1.24 g (3.38 mmol, 98% yield) of the title compound. MS (DCI/NH₃) m/z 368 (M+H)⁺.

### EXAMPLE 11C

### 5-(6-Biphenyl-3-yl-pyridin-3-yl)-hexahydro-pyrrolo[3,4-c]pyrrole-2-carboxylic Acid Tert-Butyl Ester

A mixture of the product of Example 11B (0.15 g, 0.41 mmol), 3-biphenylboronic acid (Aldrich, 85 mg, 0.43 mmol), tris(dibenzylideneacetone)dipalladium (0) (Pd₂(dba)₃, Strem, 5.6 mg, 6.1 μmol), Bu₃P (Strem, 50 μL of 10 wt % in hexanes, 17 μmol), and Cs₂CO₃ (0.16 g, 0.49 mmol) in 15 mL p-dioxane in a sealed tube was a warmed to 80° C. for 18 h. The reaction was incomplete at this point, so an equal amount to what had previously been added of 3-biphenylboronic acid, Pd₂(dba)₃, Bu₃P and Cs₂CO₃ were added and the mixture stirred at 85° C. for an additional 24 h. The mixture was cooled to ambient temperature, filtered and concentrated under reduced pressure. Purification via flash column chromatography (SiO₂, 40% hexanes −10% EtOAc −50% CH₂Cl₂) gave 0.11 g of the title compound (0.25 mmol, 61% yield). MS (DCI/NH₃) m/z 442 (M+H)⁺.

### EXAMPLE 11D

### 2-(6-Biphenyl-3-yl-pyridin-3-yl)-octahydro-pyrrolo[3,4-c]pyrrole Trifluoroacetate

To the product of Example 11C (0.11 g, 0.25 mmol) in 10 mL of CH₂Cl₂ was added 5 mL trifluoroacetic acid (TFA). This mixture stirred for 30 min at ambient temperature then was concentrated under reduced pressure. The crude material was azeotroped 2×5 mL toluene, then 10 mL EtOAc was added and the title compound crystallized out of solution as the trifluoroacetate salt (0.13 g, 0.23 mmol, 91% yield). ¹H NMR (CH₃OH-d₄, 300 MHz) δ 3.33 (m, 4H), 3.63 (m, 6H), 7.39 (m, 1H), 7.48 (m, 2H), 7.67 (m, 4H), 7.79 (m, 2H), 8.07 (m, 3H); MS (DCI/NH₃) m/z 342 (M+H)⁺; Anal. calculated for C₂₃H₂₃N₃.2CF₃CO₂H: C, 56.94; H, 4.42; N, 7.38. Found: C, 56.64; H, 4.39; N, 7.09.

### EXAMPLE 12

### 2-(6-Biphenyl-3-yl-pyridin-3-yl)-5-methyl-octahydro-pyrrolo[3,4-c]pyrrole

### EXAMPLE 12A

### 2-(6-Biphenyl-3-yl-pyridin-3-yl)-5-methyl-octahydro-pyrrolo[3,4-c]pyrrole

The product of Example 11D (0.110 g, 0.19 mmol) was converted to the free base by adding 2 mL of a 10% aqueous NaOH solution and 10 mL of a 40% aqueous Na₂CO₃ solution. The free amine was then extracted 4×10 mL CH₂Cl₂, washed 1×5 mL brine, dried over Na₂SO₄, and concentrated under reduced pressure. The free amine (55 mg, 0.16 mmol) and NaBH(OAc)₃ (36 mg, 0.16 mmol) in 3 mL of 37% aqueous formaldehyde was stirred at ambient temperature for 20 h. After this time, the reaction had not gone to completion, so another equivalent of NaBH(OAc)₃ (36 mg, 0.16 mmol) was added and the mixture stirred for an additional hour. The mixture was then quenched with 5 mL saturated, aqueous NaHCO₃, extracted 3×5 mL CH₂Cl₂, dried over Na₂SO₄ and concentrated under reduced pressure. The crude material was purified via flash column chromatography (SiO₂, 5% CH₃OH—CH₂Cl₂) to give 35 mg of the title compound (0.099 mmol, 62% yield). MS (DCI/NH₃) m/z 356 (M+H)⁺.

### EXAMPLE 12B

### 2-(6-Biphenyl-3-yl-pyridin-3-yl)-5-methyl-octahydro-pyrrolo[3,4-c]pyrrole p-toluenesulfonate

To the product of Example 12A (34 mg, 0.096 mmol) in 2 mL EtOAc was added p-toluenesulfonic acid (19 mg, 0.096 mmol) in 10% CH₃OH in EtOAc. Upon stirring at ambient temperature, a precipitate formed. Filtration gave 35 mg of the title compound (0.065 mmol, 68% yield). ¹H NMR (CH₃OH-d₄, 300 MHz) δ 2.34 (s, 3H), 2.96 (s, 3H), 3.35 (m, 6H), 3.62 (m, 4H), 7.21 (m, 2H), 7.36 (m, 2H), 7.46 (m, 2H), 7.53 (m, 1H), 7.63 (ddd, J=7.8, 1.7, 1.0 Hz, 1H), 7.69 (m, 4H), 7.81 (ddd, J=7.8, 1.7, 1.0 Hz, 1H) 7.82 (m, 1H), 8.10 (dd, J=2.0, 1.4 Hz, 1H), 8.15 (br d, J=2.7 Hz, 1H); MS (DCI/NH₃) m/z 356 (M+H)⁺; Anal. calculated for C₂₄H₂₅N₃.C₇H₈O₃S.0.5H₂O: C, 69.38; H, 6.39; N, 7.83. Found: C, 69.24; H, 6.27; N, 7.78.

### EXAMPLE 13

### 2-[6-(3-Trifluoromethyl-phenyl)-pyridin-3-yl]-octahydro-pyrrolo[3,4-c]pyrrole

### EXAMPLE 13A

### 5-(6-Chloro-pyridin-3-yl)-hexahydro-pyrrolo[3,4-c]pyrrole-2-carboxylic Acid Tert-Butyl Ester

The product of 6C (5 g, 23.6 mmol), 5-bromo-2-chloropyridine (Aldrich, 5.02 g, 28.3 mmol), tris(dibenzylideneacetone)dipalladium (0) (Pd₂(dba)₃, Strem, 0.43 g, 0.47 mmol), 2,2′-bis(diphenylphosphino)-1,1′-binaphthyl (BINAP, Strem, 0.59 g, 0.94 mmol) and tert-BuONa (3.63 g, 37.8 mmol) in 50 mL toluene was warmed to 85° C. and allowed to stir for 20 h. The mixture was cooled to ambient temperature, filtered and concentrated under reduced pressure. The crude material was purified via flash column chromatography (SiO₂, 50% hexanes-EtOAc) to give 3.86 g of the title compound (12 mmol, 42% yield) as the major product and 1.15 g of 5-(5-bromopyridin-2-yl)-hexahydro-pyrrolo[3,4-c]pyrrole-2-carboxylic acid tert-butyl ester as the minor product (3.13 mmol, 13.3% yield). MS (DCI/NH₃) m/z 324 (M+H)⁺ for major product and 367 (M+H)⁺ for the minor product.

### EXAMPLE 13B

### 5-[6-(3-Trifluoromethyl-phenyl)-pyridin-3-yl]-hexahydro-pyrrolo[3,4-c]pyrrole-2-carboxylic Acid Tert-Butyl Ester

The major product of Example 13A (0.20 g, 0.62 mmol), 3-(trifluoromethyl)phenylboronic acid (Aldrich, 0.13 g, 0.68 mmol), tris(dibenzylideneacetone)dipalladium (0) (Pd₂(dba)₃, Strem, 9 mg, 9.3 μmol), Bu₃P (Strem, 70 μL of 10 wt % in hexanes, 24 μmol), and Cs₂CO₃ (0.24 g, 0.74 mmol) were combined in 15 mL dioxane in a pressure tube. This mixture was warmed to 95° C. for 18 h after which time the reaction was incomplete and equal amounts to what had been added initially of 3-(trifluoromethyl)phenylboronic acid, Pd₂(dba)₃, Bu₃P, and Cs₂CO₃ were added again. This mixture stirred for an additional 18 h then was cooled to ambient temperature, filtered through Celite® diatomaceous earth and concentrated under reduced pressure. The crude material was purified via flash column chromatography (SiO₂, 70% hexanes-EtOAc) to give 0.11 g of the title compound (0.25 mmol, 41% yield). MS (DCI/NH₃) m/z 434 (M+H)⁺.

### EXAMPLE 13C

### 2-[6-(3-Trifluoromethyl-phenyl)-pyridin-3-yl]-octahydro-pyrrolo[3,4-c]pyrrole Trifluoroacetate

The product of Example 13B (0.11 g, 0.25 mmol) in 3 mL CH₂Cl₂ was treated with 2 mL trifluoroacetic acid (TFA) as described in Example 11D to give 70 mg of the title compound (0.14 mmol, 54% yield). ¹H NMR (CH₃OH-d₄, 300 MHz) δ 3.28 (m, 4H), 3.56 (m, 6H), 7.37 (dd, J=8.8, 3.1 Hz, 1H), 7.67 (m, 2H), 7.87 (d, J=8.8 Hz, 1H), 8.10 (m, 2H), 8.18 (m, 1H); MS (DCI/NH₃) m/z 334 (M+H)⁺; Anal. calculated for C₁₈H₁₈F₃N₃.1.6CF₃CO₂H: C, 49.37; H, 3.83; N, 8.15. Found: C, 49.44; H, 3.71; N, 8.07.

### EXAMPLE 14

### 2-Methyl-5-[6-(3-trifluoromethyl-phenyl)-pyridin-3-yl]-octahydro-pyrrolo[3,4-c]pyrrole

### EXAMPLE 14A

### 2-Methyl-5-[6-(3-trifluoromethyl-phenyl)-pyridin-3-yl]-octahydro-pyrrolo[3,4-c]pyrrole

The product of Example 13C (51 mg, 0.099 mmol) was converted to the corresponding free base and treated with 37% aqueous formaldehyde (4 mL) and NaBH(OAc)₃ (36 mg, 0.16 mmol) as described in Example 12A to give 33.7 mg of the title compound (0.097 mmol, 98% yield). MS (DCI/NH₃) m/z 348 (M+H)⁺.

### EXAMPLE 14B

### 2-Methyl-5-[6-(3-trifluoromethyl-phenyl)-pyridin-3-yl]-octahydro-pyrrolo[3,4-c]pyrrole L-tartrate

To the product of Example 14A (33.7 mg, 0.097 mmol) in 2 mL 10% CH₃OH in EtOAc was added 29 mg of L-tartaric acid (0.194 mmol) in 1 mL 10% CH₃OH in EtOAc. The resulting precipitate was isolated via filtration to give 37.8 mg of the title compound (0.057 mmol, 59% yield). ¹H NMR (CH₃OH-d₄, 300 MHz) δ 2.93 (s, 3H), 3.34 (m, 6H) 3.65 (m, 4H), 4.44 (s, 4H), 7.30 (dd, J=8.8, 3.1 Hz, 1H), 7.63 (m, 2H), 7.81 (d, J=8.8 Hz, 1H), 8.12 (m, 1H), 8.17 (br d, J=3.1 Hz, 1H) 8.19 (m, 1H); MS (DCI/NH₃) m/z 348 (M+H)⁺; Anal. calculated for C₁₉H₂₀F₃N₃.2.1C₄H₆O₆: C, 49.67; H, 4.96; N, 6.34. Found: C, 49.45; H, 5.24; N, 6.08.

### EXAMPLE 15

### 3-[5-(Hexahydro-pyrrolo[3,4-c]pyrrol-2-yl)-pyridin-2-yl]-phenylamine

### EXAMPLE 15A

### 5-[6-(3-Amino-phenyl)-pyridin-3-yl]-hexahydro-pyrrolo[3,4-c]pyrrole-2-carboxylic Acid Tert-Butyl Ester

The major product of Example 13A (0.20 g, 0.62 mmol), 3-aminobenzeneboronic acid (Alfa, 0.17 g, 1.24 mmol), tris(dibenzylideneacetone)dipalladium (0) (Pd₂(dba)₃, Strem, 40 mg, 0.044 mmol), 1,3-bis(2,6-di-1-propylphenyl)imidazolium chloride (Strem, 52 mg, 0.12 mmol), and Cs₂CO₃ (0.61 g, 1.9 mmol) in 15 mL dioxane in a sealed tube were warmed to 85° C. for 18 h. The mixture was cooled to ambient temperature, filtered through Celite® diatomaceous earth, concentrated under reduced pressure and purified via flash column chromatography (SiO₂, 80% EtOAc/hexanes) to give 0.137 g of the title compound (0.36 mmol, 58% yield). MS (DCI/NH₃) m/z 381 (M+H)⁺.

### EXAMPLE 15B

### 3-[5-(Hexahydro-pyrrolo[3,4-c]pyrrol-2-yl)-pyridin-2-yl]-phenylamine Trifluoroacetate

The product of Example 15A (0.137 g, 0.36 mmol) was treated with 3 mL CH₂Cl₂ and 2 mL trifluoroacetic acid (TFA) as in Example 11D to give 0.17 g of the title compound (0.29 mmol, 80% yield). ¹H NMR (CH₃OH-d₄, 300 MHz) δ 3.27 (m, 1H), 3.34 (m, 3H) 3.57 (m, 4H), 3.64 (m, 2H), 7.07 (ddd, J=7.8, 2.4, 1.7 Hz, 1H), 7.37 (m, 2H), 7.42 (m, 1H), 7.62 (dd, J=9.1, 3.1 Hz, 1H), 7.95 (d, J=8.8 Hz, 1H), 8.00 (d, J=2.7 Hz, 1H); MS (DCI/NH₃) m/z 281 (M+H)⁺; Anal. calculated for C₁₇H₂₀N₄.2.7CF₃CO₂H: C, 45.74; H, 3.89; N, 9.52. Found: C, 45.86; H, 3.90; N, 9.69.

### EXAMPLE 16

### 5-(6-Furan-3-yl-pyridin-3-yl)-hexahydro-pyrrolo[3,4-c]pyrrole

### EXAMPLE 16A

### 5-(6-Furan-3-yl-pyridin-3-yl)-hexahydro-pyrrolo[3,4-c]pyrrole-2-carboxylic Acid Tert-Butyl Ester

The product of Example 13A (0.20 g, 0.62 mmol), 3-furylboronic acid (Maybridge, 0.14 g, 1.24 mmol), tris(dibenzylideneacetone)dipalladium (0) (Pd₂(dba)₃, Strem, 40 mg, 0.044 mmol), 1,3-bis(2,6-di-1-propylphenyl)imidazolium chloride (Strem, 52 mg, 0.12 mmol), and Cs₂CO₃ (0.61 g, 1.9 mmol) in 15 mL dioxane and reacted as in Example 15A to give 0.17 g of the title compound (0.48 mmol, 77% yield). MS (DCI/NH₃) m/z 356 (M+H)⁺.

### EXAMPLE 16B

### 5-(6-Fu ran-3-yl-pyridin-3-yl)-hexahydro-pyrrolo[3,4-c]pyrrole Trifluoroacetate

The product of Example 16A (0.17 g, 0.48 mmol) was treated with 7 mL CH₂Cl₂ and 5 mL trifluoroacetic acid (TFA) as in Example 11D to give 0.195 g of the title compound (0.40 mmol, 84% yield). ¹H NMR (CH₃OH-d₄, 300 MHz) δ 3.25 (m, 2H), 3.33 (m, 2H), 3.55 (m, 4H), 3.64 (m, 2H), 6.97 (dd, J=2.0, 1.0 Hz, 1H), 7.60 (dd, J=9.2, 3.1 Hz, 1H), 7.70 (dd, J=1.7, 1.7 Hz, 1H), 7.87 (d, J=9.2 Hz, 1H), 7.91 (d, J=2.7 Hz, 1H), 8.19 (dd, J=1.4, 1.0 Hz, 1H). MS (DCI/NH₃) m/z 256 (M+H)⁺; Anal. calculated for C₁₅H₁₇N₃0.2CF₃CO₂H: C, 47.21; H, 3.96; N, 8.69. Found: C, 47.17; H, 4.01; N, 8.65.

### EXAMPLE 17

### 2-(6-Furan-3-yl-pyridin-3-yl)-5-methyl-octahydro-pyrrolo[3,4-c]pyrrole

### EXAMPLE 17A

### 2-(6-Furan-3-yl-pyridin-3-yl)-5-methyl-octahydro-pyrrolo[3,4-c]pyrrole

The product of Example 16B (0.16 g, 0.33 mmol) was treated with NaBH(OAc)₃ (98 mg, 0.46 mmol) in 5 mL 37% aqueous formaldehyde. This mixture stirred at ambient temperature for 2 h then was quenched with 5 mL NaHCO₃. CH₂Cl₂ (5 mL) was added, the layers were separated, the aqueous layer was extracted 3×5 mL CH₂Cl₂. The combined organics were dried over Na₂SO₄ and concentrated to give the title compound. MS (DCI/NH₃) m/z 269 (M+H)⁺.

### EXAMPLE 17B

### 2-(6-Fu ran-3-yl-pyridin-3-yl)-5-methyl-octahydro-pyrrolo[3,4-c]pyrrole p-toluenesulfonate

The product of Example 17A (0.33 mmol) and p-toluenesulfonic acid (66 mg, 0.33 mmol) were combined as in Example 12B to give 83 mg of the title compound (0.157 mmol, 48% yield). ¹H NMR (CH₃OH-d₄, 300 MHz) δ 2.35 (s, 4H) 2.95 (m, 4H), 3.29 (m, 3H), 3.42 (m, 3H), 3.64 (m, 3H), 6.93 (dd, J=2.0, 1.0 Hz, 1H), 7.21 (m, 3H), 7.47 (m, 1H), 7.64 (dd, J=1.7, 1.7 Hz, 1H), 7.68 (m, 3H), 7.72 (m, 1H), 7.97 (m, 1H), 8.11 (dd, J=1.4, 1.0 Hz, 1H). MS (DCI/NH₃) m/z 270 (M+H)⁺; Anal. calculated for C₁₅H₁₇N₃O.1.5C₇H₈O₂S: C, 60.32; H, 5.92; N, 7.96. Found: C, 60.34; H, 6.00; N, 8.11.

### EXAMPLE 18

### 2-(6-Benzo[b]thiophen-2-yl-pyridin-3-yl)-octahydro-pyrrolo[3,4-c]pyrrole

### EXAMPLE 18A

### 5-(6-Benzo[b]thiophen-2-yl-pyridin-3-yl)-hexahydro-pyrrolo[3,4-c]pyrrole-2-carboxylic Acid Tert-Butyl Ester

The major product of Example 13A (0.20 g, 0.62 mmol), 2-benzothiophene-2-boronic acid (Aldrich, 0.14 g, 1.24 mmol), tris(dibenzylideneacetone)dipalladium (0) (Pd₂(dba)₃, Strem, 20 mg, 0.022 mmol), Bu₃P (Strem, 150 μL of 10 wt % in hexanes, 51 μmol), and Cs₂CO₃ (0.61 g, 1.9 mmol) in 15 mL dioxane and reacted as in Example 13B to give 60 mg of the title compound (0.14 mmol, 23% yield). MS (DCI/NH₃) m/z 422 (M+H)⁺.

### EXAMPLE 18B

### 2-(6-Benzo[b]thiophen-2-yl-pyridin-3-yl)-octahydro-pyrrolo[3,4-c]pyrrole Trifluoroacetate

The product of Example 18A (60 mg, 0.14 mmol) was treated with 5 mL CH₂Cl₂ and 5 mL trifluoroacetic acid (TFA) as in Example 11D to give the title compound. ¹H NMR (CH₃OH-d₄, 300 MHz) δ 3.26 (m, 4H), 3.45 (m, 2H), 3.55 (m, 2H), 3.63 (m, 2H), 7.21 (dd, J=8.8, 3.1 Hz, 1H), 7.32 (m, 2H), 7.73 (br s, 1H), 7.78 (m, 1H), 7.83 (m, 2H), 8.03 (br d, J=2.7 Hz, 1H); MS (DCI/NH₃) m/z 322 (M+H)⁺; Anal. calculated for C₁₉H₁₉N₃S.1.1CF₃CO₂H: C, 56.98; H, 4.53; N, 9.40. Found: C, 57.11; H, 4.44; N, 9.21.

### EXAMPLE 19

### 2-(6-Benzo[b]thiophen-2-yl-pyridin-3-yl)-5-methyl-octahydro-pyrrolo[3,4-c]pyrrole

### EXAMPLE 19A

### 2-(6-Benzo[b]thiophen-2-yl-pyridin-3-yl)-5-methyl-octahydro-pyrrolo[3,4-c]pyrrole

To the product of Example 18B (50 mg, 0.11 mmol) in 5 mL of 37% aqueous formaldehyde was added NaBH(OAc)₃ (27 mg, 0.13 mmol). This mixture stirred at ambient temperature for 18 h, then was quenched with 5 mL saturated, aqueous NaHCO₃. The layers were separated and the aqueous layer was extracted 4×5 mL of CH₂Cl₂. The combined organic layers were dried over Na₂SO₄, concentrated under reduced pressure and purified by flash column chromatography (SiO₂, 1% NH₄OH: 9% CH₃OH:90% CH₂Cl₂) to give 17 mg of the title compound (0.051 mmol, 46% yield) which was carried on directly to the next reaction.

### EXAMPLE 19B

### 2-(6-Benzo[b]thiophen-2-yl-pyridin-3-yl)-5-methyl-octahydro-pyrrolo[3,4-c]pyrrole p-toluenesulfonate

The product of Example 19A (17 mg, 0.051 mmol) and p-toluenesulfonic acid (11 mg, 0.058 mmol) were reacted as in Example 12B to give 12 mg of the title compound (0.021 mmol, 42% yield). ¹H NMR (CH₃OH-d₄, 300 MHz) δ 2.36 (s, 3H), 2.93 and 2.98 (rotamer s, 3H), 3.27 (m, 2H), 3.29 (m, 2H), 3.42 (m, 2H), 3.65 (m, 3H), 3.98 (m, 1H), 7.22 (m, 2H), 7.32 (m, 3H), 7.70 (m, 2H), 7.75 (br s, 1H), 7.78 (m, 1H), 7.84 (dd, J=7.1, 2.0 Hz, 1H), 7.85 (br d, J=8.5 Hz, 1H), 8.07 (br d, J=2.4 Hz, 1H); MS (DCI/NH₃) m/z 322 (M+H)⁺; Anal. calculated for C₂₀H₂₁N₃s.1.2C₇H₈O₃S.H₂O: C, 60.90; H, 5.87; N, 7.50. Found: C, 60.93; H, 5.74; N, 7.31.

### EXAMPLE 20

### 2-(5-Phenyl-pyridin-2-yl)-octahydro-pyrrolo[3,4-c]pyrrole

### EXAMPLE 20A

### 5-(5-Phenyl-pyridin-2-yl)-hexahydro-pyrrolo[3,4-c]pyrrole-2-carboxylic Acid Tert-Butyl Ester

A mixture of 5-(5-bromopyridin-2-yl)-hexahydro-pyrrolo[3,4-c]pyrrole-2-carboxylic acid tert-butyl ester from Example 13A (0.25 g, 0.68 mmol), phenylboronic acid (0.26 g, 1.36 mmol), tris(dibenzylideneacetone)dipalladium (0) (Pd₂(dba)₃, Strem, 40 mg, 0.044 mmol), 1,3-bis(2,6-di-1-propylphenyl)imidazolium chloride (Strem, 52 mg, 0.12 mmol), and Cs₂CO₃ (0.67 g, 1.9 mmol) in 15 mL dioxane were reacted as in Example 15A to give 0.20 g of the title compound (0.55 mmol, 80% yield). MS (DCI/NH₃) m/z 366 (M+H)⁺.

### EXAMPLE 20B

### 2-(5-Phenyl-pyridin-2-yl)-octahydro-pyrrolo[3,4-c]pyrrole Trifluoroacetate

The product of Example 20A (0.20 g, 0.55 mmol) in 5 mL CH₂Cl₂ was treated with 5 mL TFA as described in Example 11D to give 0.233 g of the title compound (0.46 mmol, 84% yield). ¹H NMR (CH₃OH-d₄, 300 MHz) δ 3.33 (br d, J=4.4 Hz, 1H), 3.37 (m, 1H), 3.44 (m, 1H), 3.70 (m, 4H), 3.92 (m, 2H), 7.08 (d, J=9.5 Hz, 1H), 7.52 (m, 3H), 7.65 (m, 2H), 8.18 (br d, J=2.4 Hz, 1H), 8.25 (dd, J=9.5, 2.4 Hz, 1H); MS (DCI/NH₃) m/z 266 (M+H)⁺; Anal. calculated for C₂₀H₂₁N₃S.2.1CF₃CO₂H: C, 50.44; H, 4.21; N, 8.32. Found: C, 50.57; H, 4.38; N, 8.32.

### EXAMPLE 21

### 2-Methyl-5-(5-phenyl-pyridin-2-yl)-octahydro-pyrrolo[3,4-c]pyrrole

### EXAMPLE 21A

### 2-Methyl-5-(5-phenyl-pyridin-2-yl)-octahydro-pyrrolo[3,4-]pyrrole

The product of Example 20B (0.20 g, 0.40 mmol) in 7 mL 37% aqueous formaldehyde was treated with NaBH(OAc)₃ (0.21 g, 0.57 mmol) as described in Example 19A to give 74 mg of the title compound (0.265 mmol, 66% yield).

### EXAMPLE 21B

### 2-Methyl-5-(5-phenyl-pyridin-2-yl)-octahydro-pyrrolo[3,4-c]pyrrole p-toluenesulfonate

The product of Example 21A (74 mg, 0.265 mmol) was treated with p-toluenesulfonic acid (53 mg, 0.28 mmol) as described in Example 12B to give 85 mg of the title compound (0.18 mmol, 69% yield). ¹H NMR (CH₃OH-d₄, 300 MHz) δ 2.35 (s, 3H), 2.96 (s, 3H), 3.22 (m, 2H), 3.43 (m, 3H), 3.66 (m, 4H), 3.98 (m, 1H), 6.82 (d, J=9.2 Hz, 1H), 7.22 (m, 2H), 7.34 (tt, J=7.1, 2.0 Hz, 1H), 7.44 (m, 2H), 7.56 (m, 2H), 7.68 (m, 2H), 7.92 (dd, J=8.8, 2.4 Hz, 1H), 8.32 (d, J=2.0 Hz, 1H); MS (DCI/NH₃) m/z 280 (M+H)⁺; Anal. calculated for C₂₀H₂₁N₃S.C₇H₈O₃S.0.6H₂O: C, 64.94; H, 6.58; N, 9.09. Found: C, 65.00; H, 6.50; N, 8.71.

### EXAMPLE 22

### 2-(2-Phenyl-pyrimidin-5-yl)-octahydro-pyrrolo[3,4-c]pyrrole

### EXAMPLE 22A

### 5-Pyrimidin-5-yl-hexahydro-pyrrolo[3,4-c]pyrrole-2-carboxylic Acid Tert-Butyl Ester

A mixture of the product of Example 6C (2.045 g, 9.63 mmol), 5-bromopyrimidine (1.84 g, 11.6 mmol), tris(dibenzylideneacetone)dipalladium (0) Pd₂(dba)₃, Strem, 0.265 g, 0.29 mmol), racemic-2,2′-bis(diphenylphosphino)-1,1′-binaphthyl (BINAP, Strem, 0.30 g, 0.48 mmol) and tert-BuONa (sodium tert-butoxide, 1.85 g, 19.3 mmol) in 75 mL PhCH₃ was degassed three times with a N₂ back-flush. The mixture was warmed to 85° C., stirred for 48 h then was cooled, filtered and concentrated under reduced pressure. Purification by column chromatography (SiO₂, 50% hexanes-EtOAc) gave 2.68 g of the title compound (9.23 mmol, 95% yield). MS (DCI/NH₃) m/z 291 (M+H)⁺.

### EXAMPLE 22B

### 5-(2-Bromo-pyrimidin-5-yl)-hexahydro-pyrrolo[3,4-c]pyrrole-2-carboxylic Acid Tert-Butyl Ester

To a solution of the product of Example 22A (2.68 g, 9.23 mmol) in 75 mL of CH₃CN at 0° C. was added N-bromosuccinimide (NBS, 1.64 g, 9.23 mmol) in 50 mL CH₃CN portionwise via cannula. The mixture was allowed to warm to ambient temperature and stir for 16 h. The reaction mixture was quenched by the addition of 25 mL H₂O then 50 mL CH₂Cl₂ was added. The layers were separated and the aqueous layer was extracted 3×20 mL CH₂Cl₂. The combined organic layers were washed with 10 mL saturated, aqueous NaCl (brine), then were dried over Na₂SO₄, and concentrated under reduced pressure. Purification via column chromatography (SiO₂, 75% hexanes-EtOAc) gave 1.2 g of the title compound (3.25 mmol, 35% yield). MS (DCI/NH₃) m/z 369, 371 (M+H)⁺.

### EXAMPLE 22C

### 5-(2-Phenyl-pyrimidin-5-yl)-hexahydro-pyrrolo[3,4-c]pyrrole-2-carboxylic Acid Tert-Butyl Ester

The product of Example 22B (0.42 g, 1.14 mmol), phenylboronic acid (Aldrich, 0.28 g, 2.27 mmol), tris(dibenzylideneacetone)dipalladium (0) Pd₂(dba)₃, Strem, 42 mg, 0.046 mmol), 1,3-bis(2,6-di-1-propylphenyl)imidazolium chloride (Strem, 48 mg, 0.11 mmol), and 2 mL of 2M Na₂CO₃ in H₂O were combined in 20 mL PhCH₃. This mixture was degassed three times with a N₂ back-flush then was warmed to 85° C. and allowed to stir for 16 h. The mixture was cooled to ambient temperature, filtered, concentrated under reduced pressure and purified via column chromatography (SiO₂, 50% hexanes:EtOAc) to give 0.28 g of the title compound (0.76 mmol, 67% yield). MS (DCI/NH₃) m/z 367 (M+H)⁺.

### EXAMPLE 22D

### 2-(2-Phenyl-pyrimidin-5-yl)-octahydro-pyrrolo[3,4-c]pyrrole

The product of Example 22C (0.28 g, 0.76 mmol) in 7 mL CH₂Cl₂ was treated with 4 mL trifluoroacetic acid (TFA) as described in Example 1K to give the title compound which was used in the next step without further purification.

### EXAMPLE 22E

### 2-(2-Phenyl-pyrimidin-5-yl)-octahydro-pyrrolo[3,4-c]pyrrole p-toluenesulfonate

The product of Example 22D (55 mg, 0.21 mmol) and p-toluenesulfonic acid (p-TsOH.H₂O, 39 mg, 0.21 mmol) were combined as in Example 1L to give 0.10 g of the title compound (0.21 mmol, 100% yield). ¹H NMR (CH₃OH-d₄, 300 MHz) δ 2.35 (s, 3H), 3.27 (m, 4H), 3.45 (m, 2H), 3.59 (m, 4H), 7.20 (m, 2H), 7.42 (m, 3H), 7.69 (m, 2H), 8.22 (m, 2H), 8.30 (s, 2H); MS (DCI/NH₃) m/z 267 (M+H)⁺; Anal. calculated for C₁₆H₁₈N₄.1.25C₇H₈O₃S: C, 61.73; H, 5.86; N, 11.63; Found: C, 61.47; H, 5.85; N, 11.71.

### EXAMPLE 23

### 2-Methyl-5-(2-phenyl-pyrimidin-5-yl)-octahydro-pyrrolo[3,4-c]pyrrole

### EXAMPLE 23A

### 2-Methyl-5-(2-phenyl-pyrimidin-5-yl)-octahydro-pyrrolo[3,4-c]pyrrole

To the product of Example 22D (0.148 g, 0.55 mmol) in 5 mL HCHO (37% aqueous solution) was added 1 mL 1,2-dichloroethane (for solubility) and 0.165 g of NaBH(OAc)₃ (0.78 mmol). This mixture stirred at ambient temperature for 3 h then was quenched with 5 mL saturated, aqueous NaHCO₃ and diluted with 5 mL CH₂Cl₂. The layers were separated, the aqueous layer was extracted 3×5 mL CH₂Cl₂ and the combined organics were dried over Na₂SO₄, concentrated under reduced pressure and purified via column chromatography (SiO₂, 1% NH₄OH: 9% CH₃OH:90% CH₂Cl₂) to give 0.15 g of the title compound (0.535 mmol, 97% yield). MS (DCI/NH₃) m/z 281 (M+H)⁺.

### EXAMPLE 23B

### 2-Methyl-5-(2-phenyl-pyrimidin-5-yl)-octahydro-pyrrolo[3,4-c]pyrrole p-toluenesulfonate

The product of Example 23A (0.15 g, 0.535 mmol) and p-toluenesulfonic acid (p-TsOH.H₂O, 0.102 g, 0.535 mmol) were combined as in Example 1L to give 0.23 g of the title compound (0.51 mmol, 95% yield). ¹H NMR (CH₃OH-d₄, 300 MHz) δ 2.35 (s, 3H), 2.95 (s, 3H), 3.30 (m, 2H), 3.35 (m, 4H), 3.65 (m, 4H), 7.21 (m, 2H), 7.43 (m, 3H), 7.69 (m, 2H), 8.23 (m, 2H), 8.36 (s, 2H); MS (DCI/NH₃) m/z 281 (M+H)⁺; Anal. calculated for C₁₇H₂₀N₄.C₇H₈O₃S: C, 63.69; H, 6.24; N, 12.38; Found: C, 63.32; H, 6.12; N, 12.07.

### EXAMPLE 24

### Diethyl-(2-{3-[6-(hexahydro-pyrrolo[3,4-c]pyrrol-2-yl)-pyridazin-3-yl]-phenoxy}-ethyl)-amine di-L-tartrate

### EXAMPLE 24A

### 5-[6-(3-Methoxy-phenyl)-pyridazin-3-yl]-hexahydro-pyrrolo[3,4-c]pyrrole-2-carboxylic Acid Tert-Butyl Ester

The product of Example 8A (0.5 g, 1.54 mmol), m-methoxyphenylboronic acid (Aldrich, 0.47 g, 3.1 mmol), tris(dibenzylideneacetone)dipalladium (0) (Pd₂ dba₃, Strem, 56 mg, 0.062 mmol), 1,3-bis(2,6-di-1-propylphenyl)imidazolium chloride (Nolan's catalyst, Strem, 65 mg, 0.154 mmol) and 2.5 mL of 2N Na₂CO₃ were combined in 25 mL toluene and reacted as in Example 10A to give 0.51 g of the title compound (1.29 mmol, 84% yield). MS (DCI/NH₃) m/z 397 (M+H)⁺.

### EXAMPLE 24B

### 3-[6-(Hexahydro-pyrrolo[3,4-c]pyrrol-2-yl)-pyridazin-3-yl]-phenol

To the product of Example 24A (0.40 g, 1.01 mmol) in 50 mL CH₂Cl₂ at −78° C. was added 4.04 mL of a 1M BBr₃ solution (4.8 mmol). This mixture was stirred at −78° C. for 30 min then was warmed to ambient temperature and was allowed to stir for 18 hours. The reaction was quenched via addition of 10 mL H₂O and was concentrated under reduced pressure. Purification of the crude material via column chromatography (SiO₂, 1% NH₄OH:9% CH₃OH:90% CH₂Cl₂) gave 0.26 g of the title compound (0.92 mmol, 91% yield). MS (DCI/NH₃) m/z 283 (M+H)⁺.

### EXAMPLE 24C

### 5-[6-(3-Hydroxy-phenyl)-pyridazin-3-yl]-hexahydro-pyrrolo[3,4-c]pyrrole-2-carboxylic Acid Tert-Butyl Ester

To the product of Example 24A (0.26 g, 0.92 mmol) in 40 mL THF was added 1 mL saturated, aqueous NaHCO₃ solution followed by di-tert-butyl dicarbonate (0.21 g, 0.97 mmol). This mixture stirred at ambient temperature for 4 h then was diluted with 30 mL CH₂Cl₂ and 20 mL H₂O. The layers were separated and the aqueous layer was extracted 2×10 mL CH₂Cl₂. The combined organics were dried over anhydrous Na₂SO₄, concentrated under reduced pressure and purified by column chromatography (SiO₂, 50% hexanes-EtOAc) to give 0.32 g of the title compound (0.84 mmol, 91% yield). MS(DCI/NH₃) m/z 383 (M+H)⁺.

### EXAMPLE 24D

### 5-[6-[3-(2-Diethylamino-ethoxy)-phenyl]-pyridazin-3-yl]-hexahydro-pyrrolo[3,4-c]pyrrole-2-carboxylic Acid Tert-Butyl Ester

To a solution of the product of Example 24C (0.1 g, 0.26 mmol) and N,N-diethylethanolamine (Aldrich, 87 mL, 0.65 mmol) in 10 mL CH₂Cl₂ at 0° C. was added polymer-supported triphenylphosphine (Aldrich, 3 mmol/gram, 0.65 mmol, 0.22 g). The diisopropyl azodicarboxylate (DIAD, Aldrich, 0.13 mL, 0.65 mmol) was added dropwise via syringe and, following the addition, the ice-bath was removed and the reaction mixture stirred at ambient temperature for 2 h. The mixture was then filtered, concentrated and purified via column chromatography (SiO₂, 1% NH₄OH:9% CH₃OH:90% CH₂Cl₂) to give 0.12 g of the title compound (0.25 mmol, 96% yield). MS (DCI/NH₃) m/z 482 (M+H)⁺.

### EXAMPLE 24E

### Diethyl-(2-[3-{6-(hexahydro-pyrrolo[3,4-c]pyrrol-2-yl)-pyridazin-3-yl]-phenoxy}-ethyl)-amine

The product of Example 24D (0.12 g, 0.25 mmol) 3 mL CH₂Cl₂ at 0° C. was added 2 mL trifluoroacetic acid (TFA). The ice-bath was removed after addition of the TFA and the reaction mixture stirred at ambient temperature for 2 h. Concentration under reduced pressure followed by column chromatography gave 60 mg of the title compound (0.16 mmol, 63% yield).

### EXAMPLE 24F

### Diethyl-(2-[3-[6-(hexahydro-pyrrolo[3,4-c]pyrrol-2-yl)-pyridazin-3-yl]-phenoxy]ethyl)-amine di-L-tartrate

To the product of Example 24E (16.8 mg, 0.044 mmol) in 2 mL EtOAc was added L-tartaric acid (8.4 mg, 0.044 mmol) in 10% CH₃OH in EtOAc. The resulting precipitate was isolated via filtration to give 16 mg of the title compound (0.021 mmol, 47% yield). ¹H NMR (CH₃OH-d₄, 300 MHz) δ 1.37 (t, J=7.1 Hz, 6H), 3.34 (m, 8H) 3.69 (m, 9H), 4.40 (s, 4H, tartrate), 4.44 (br t, J=5.1 Hz, 2H), 7.08 (ddd, J=8.1, 2.4, 0.7 Hz, 1H), 7.13 (d, J=9.5 Hz, 1H), 7.44 (dd, J=8.1, 8.1 Hz, 1H), 7.54 (m, 1H), 7.65 (dd, J=1.7, 1.7 Hz, 1H), 7.90 (d, J=9.5 Hz, 1H); MS (DCI/NH₃) m/z 382 (M+H-2.6C₄H₆O₆.0.5H₂O)⁺; Anal. calculated for C₂₂H₃₁N₅O.2.6C₄H₆O₆.0.5H₂O: C, 49.84; H, 6.15; N, 8.97. Found: C, 49.75; H, 5.56; N, 9.05.

### EXAMPLE 25

### Diethyl-(2-{3-[6-(5-methyl-hexahydro-pyrrolo[3,4-c]pyrrol-2-yl)-pyridazin-3]-phenoxy}-ethyl)-amine Difumarate

### EXAMPLE 25A

### Diethyl-(2-{3-[6-(5-methyl-hexahydro-pyrrolo[3,4-c]pyrrol-2-yl)-pyridazin-3-yl]-phenoxy}-ethyl)-amine

The product of Example 24E (43.2 mg, 0.11 mmol) was treated with NaBH(OAc)₃ (36 mg, 0.17 mmol) in 3 mL 36% aqueous formaldehyde. This mixture stirred at ambient temperature for 4 h then was quenched with 5 mL saturated, aqueous NaHCO₃. CH₂Cl₂ (5 mL) was added, the layers were separated, the aqueous layer was extracted 3×5 mL CH₂Cl₂. The combined organics were dried over Na₂SO₄, concentrated and purified to give 40 mg of the title compound (0.10 mmol, 89% yield).

### EXAMPLE 25B

### Diethyl-(2-{3-[6-(5-methyl-hexahydro-pyrrolo[3,4-c]pyrrol-2-yl)-pyridazin-3yl]-phenoxy}-ethyl)-amine-difumarate

To the product of Example 25A (40 mg, 0.10 mmol) in 2 mL of 10% CH₃OH in diethyl ether was added fumaric acid (23.2 mg, 0.20 mmol) in 2 mL of 10% CH₃OH in diethyl ether. The resulting precipitate was isolated via filtration to give 44 mg of the title compound (0.066 mmol, 66% yield). ¹H NMR (CH₃OH-d₄, 300 MHz) δ 1.37 (t, J=7.1 Hz, 6H), 2.89 (s, 3H), 3.30 (m, 4H), 3.35 (q, J=7.1 Hz, 4H), 3.58 (m, 3H); 3.63 (dd, J=4.8, 4.8 Hz, 2H), 3.75 (m, 3H), 4.44 (dd, J=4.8, 4.8 Hz, 2H), 6.7 (s, 4H), 7.09 (ddd, J=8.1, 2.4, 0.7 Hz, 1H), 7.15 (d, J=9.5 Hz, 1H), 7.45 (dd, J=8.1, 8.1 Hz, 1H), 7.54 (m, 1H), 7.65 (dd, J=1.7, 1.7 Hz, 1H), 7.91 (d, J=9.5 Hz, 1H); MS (DCI/NH₃) m/z 396 (M+H-2C₄H₄O₄-1 NH₅O)⁺; Anal. calculated for C₂₃H₃₃N₅O-2C₄H₄O₄.NH₅O: C, 56.18; H, 7.00; N, 12.68. Found: C, 56.40; H, 6.50; N, 12.94.

### EXAMPLE 26

### 2-(5-Phenyl-[1,3,4]thiadiazol-2-yl)-octahydro-pyrrolo[3,4-c]pyrrole

### EXAMPLE 26A

### 2-Bromo-5-phenyl-[1,3,4]thiadiazole

The 2-amino-5-phenyl-1,3,4-thiadiazole sulfate (Aldrich, 2.5 g, 9.08 mmol) and 10 mL 48% aqueous HBr were combined in a 3-neck, 250 mL round-bottomed flask equipped with a stir bar, addition funnel and temperature probe. This mixture was cooled to 5° C. and NaNO₂ (0.69 g, 9.99 mmol) in 10 mL H₂O was added dropwise via addition funnel with the internal temperature being maintained at approximately 5° C. The mixture stirred for 15 min after the addition was complete then CuBr (0.69 g, 4.8 mmol) was added portion-wise with the temperature again being maintained at approximately 5° C. After the addition was complete, the reaction was allowed to warm to ambient temperature and was stirred for 16 h. The mixture was diluted with 20 mL CH₂Cl₂ and 10 mL H₂O. The layers were separated and the organic layer was concentrated under reduced pressure to give 1.63 g of the title compound (6.76 mmol, 74% yield). MS (DCI/NH₃) m/z 241, 243 (M+H)⁺.

### EXAMPLE 26B

### 5-(5-Phenyl-[1,3,4]thiadiazol-2-yl)-hexahydro-pyrrolo[3,4-c]pyrrole-2-carboxylic Acid Tert-Butyl Ester

The products of Examples 6C (0.70 g, 3.32 mmol) and 26A (0.88 g, 3.65 mmol) were combined with tris(dibenzylideneacetone)dipalladium (0) Pd₂(dba)₃, Strem, 0.12 g, 0.13 mmol), racemic-2,2′-bis(diphenylphosphino)-1,1′-binaphthyl (BINAP, Strem, 0.33 g, 0.21 mmol) and Cs₂CO₃ (2.2 g, 6.63 mmol) in 50 mL PhCH₃ and were reacted as in Example 22A to give 0.53 g of the title compound (1.42 mmol, 43% yield). MS (DCI/NH₃) m/z 373 (M+H)⁺.

### EXAMPLE 26C

### 2-(5-Phenyl-[1,3,4]thiadiazol-2-yl)-octahydro-pyrrolo[3,4-c]pyrrole

The product of Example 26B (0.53 g, 1.42 mmol) in 5 mL CH₂Cl₂ was treated with 2.5 mL trifluoroacetic acid (TFA) as described in Example 1K to give the title compound.

### EXAMPLE 26D

### 2-(5-Phenyl-[1,3,4]thiadiazol-2-yl)-octahydro-pyrrolo[3,4-c]pyrrole Fumarate

The product of Example 26C (1.42 mmol) was dissolved in 10 mL of 10% CH₃OH in diethyl ether. Fumaric acid in 5 mL of 10% CH₃OH in diethyl ether was added and a precipitate formed immediately. Mixture stirred at ambient temperature for 1 h then the precipitate was isolated via filtration to give 0.407 g of the title compound (1.03 mmol, 73% yield). ¹H NMR (CH₃OH-d₄, 300 MHz) δ 3.27 (m, 1H), 3.33 (m, 3H), 3.60 (m, 4H), 3.79 (m, 2H), 6.68 (s, 2H), 7.46 (m, 3H), 7.80 (m, 2H); MS (DCI/NH₃) m/z 305 (M+H)⁺; Anal. calculated for C₁₄H₁₆N₄S.C₄H₄O₄.0.3H₂O: C, 54.89; H, 5.27; N, 14.23; Found: C, 54.66; H, 6.10; N, 14.19.

### EXAMPLE 27

### 2-(3-Phenyl-[1,2,4]thiadiazol-5-yl)-octahydro-pyrrolo[3,4-c]pyrrole

### EXAMPLE 27A

### 5-(3-Phenyl-[1,2,4]thiadiazol-5-yl)-hexahydro-pyrrolo[3,4-c]pyrrole-2-carboxylic Acid Tert-Butyl Ester

5-Chloro-3-phenyl-[1,2,4]thiadiazole (0.75 g, 3.81 mmol) was prepared according to literature procedure (Goerdeler, J. et al Chem. Ber. 1957, 90, 182) and was combined with the product of 6C (0.85 g, 4.0 mmol), tris(dibenzylideneacetone)dipalladium (0) Pd₂(dba)₃, Strem, 0.105 g, 0.11 mmol), 1,3-bis(2,6-di-1-propylphenyl)imidazolium chloride (Strem, 97 mg, 0.23 mmol) and tert-BuONa (Aldrich, 0.73 g, 7.6 mmol) in 40 mL PhCH₃. This mixture was degassed three times with N₂ backflush. The reaction was warmed to 85° C. for 18 h then was cooled to ambient temperature, concentrated under reduced pressure and purified via column chromatography (SiO₂, 50% hexanes-EtOAc) to give 1.02 g of the title compound (2.74 mmol, 72% yield). MS (DCI/NH₃) m/z 373 (M+H)⁺.

### EXAMPLE 27B

### 2-(3-Phenyl-[1,2,4]thiadiazol-5-yl)-octahydro-pyrrolo[3,4-c]pyrrole

The product of Example 27A (0.15 g, 0.40 mmol) in 5 mL CH₂Cl₂ was treated with 3 mL trifluoroacetic acid (TFA) as described in Example 1K to give 0.1 g of the title compound (0.37 mmol, 92% yield). Material was carried on directly to the next reaction.

### EXAMPLE 27C

### 2-(3-Phenyl-[1,2,4]thiadiazol-5-yl)-octahydro-pyrrolo[3,4-c]pyrrole p-toluenesulfonate

The product of Example 27B (0.1 g, 0.37 mmol) and p-toluenesulfonic acid (p-TsOH.H₂O, 70 mg, 0.37 mmol) were combined as in Example 1L to give 68 mg of the title compound (0.13 mmol, 77% yield). ¹H NMR (CH₃OH-d₄, 300 MHz) δ 2.36 (s, 3H), 2.38 (m, 1H), 3.36 (m, 3H), 3.63 (m, 4H), 3.84 (m, 2H), 7.22 (m, 2H), 7.43 (m, 3H), 7.70 (m, 2H), 8.14 (m, 2H); MS (DCI/NH₃) m/z 273 (M+H)⁺; Anal. calculated for C₁₄H₁₆N₄S.1.2C₇H₈O₃S: C, 56.17; H, 5.39; N, 11.70; Found: C, 56.28; H, 5.46; N, 11.63.

### EXAMPLE 28

### 2-Methyl-5-(3-phenyl-[1,2,4]thiadiazol-5-yl)-octahydro-pyrrolo[3,4-c]pyrrole

### EXAMPLE 28A

### 2-Methyl-5-(3-phenyl-[1,2,4]thiadiazol-5-yl)-octahydro-pyrrolo[3,4-c]pyrrole

To the product of Example 27A (0.32 g, 0.86 mmol) in 6 mL CH₂Cl₂ at 0° C. was added 4 mL trifluoroacetic acid (TFA). The mixture was allowed to warm to ambient temperature and stir for 2 h. The reaction mixture was then concentrated and the residue was dissolved in 5 mL 37% aqueous HCHO. NaBH(OAc)₃ (0.26 g, 1.2 mmol) was added and the mixture stirred for 5 h at ambient temperature. The mixture was quenched with 5 mL saturated, aqueous NaHCO₃ and 5 mL of CH₂Cl₂ was added. The layers were separated and the aqueous layer was extracted 3×5 mL CH₂Cl₂. The combined organics were dried over Na₂SO₄, concentrated under reduced pressure and purified via column chromatography (SiO₂, 1% NH₄OH: 9% CH₃OH: 90% CH₂Cl₂) to give the title compound which was carried on directly to the next reaction without further purification.

### EXAMPLE 28B

### 2-Methyl-5-(3-phenyl-[1,2,4]thiadiazol-5-yl)-octahydro-pyrrolo[3,4-c]pyrrole Fumarate

The product of Example 28A (0.86 mmol) and fumaric acid (0.1 g, 0.86 mmol) were combined as in Example 26D to give 0.22 g of the title compound (0.55 mmol, 64% yield). ¹H NMR (CH₃OH-d₄, 300 MHz) δ 2.81 (s, 3H), 3.17(dd, J=11.2, 4.8 Hz, 2H), 3.36 (m, 2H), 3.48 (m, 2H), 3.63 (dd, J=11.2, 2.7 Hz, 2H), 3.78 (m, 2H), 6.69 (s, 2H), 7.44 (m, 3H), 8.13 (m, 2H); MS (DCI/NH₃) m/z 287 (M+H)⁺; Anal. calculated for C₁₅H₁₈N₄S.C₄H₄O₄: C, 56.70; H, 5.51; N, 13.92; Found: C, 56.42; H, 5.51; N, 13.71.

### EXAMPLE 29

### 2-(1-Phenyl-1H-pyrazol-4-yl)-octahydro-pyrrolo[3,4-c]pyrrole

### EXAMPLE 29A

### 4-Bromo-1-phenyl-1H-pyrazole

To 1-phenylpyrazole (Aldrich, 1 g, 6.94 mmol) in 10 mL acetic acid was added 1.1 g of Br₂ (Fisher, 6.94 mmol) in 10 mL acetic acid. This mixture was warmed to 100° C. in a pressure tube for 8 h. The material was cooled to ambient temperature, poured into ice and H₂O in a 500 mL beaker and excess saturated, aqueous NaHCO₃ was added until all the acetic acid had been quenched. EtOAc (50 mL) was added and the layers were separated. The aqueous layer was extracted 2×15 mL EtOAc and the combined organics were dried over Na₂SO₄ and concentrated under reduced pressure to give a crude solid. Purification via flash column chromatography (SiO₂, 50% hexanes-EtOAc) gave 1.5 g of the title compound (6.72 mmol, 97% yield). MS (DCI/NH₃) m/z 223, 225 (M+H)⁺.

### EXAMPLE 29B

### 5-(1-Phenyl-1H-pyrazol-4-yl)-hexahydro-pyrrolo[3,4-c]pyrrole-2-carboxylic Acid Tert-Butyl Ester

To the product of Example 6C (0.5 g, 2.4 mmol) in 15 mL toluene in a pressure tube was added the product of 29A (0.68 g, 3.06 mmol), tris(dibenzylideneacetone)dipalladium (0) (Pd₂(dba)₃, Strem, 43 mg, 0.047 mmol), racemic-2,2′-bis(diphenylphosphino)-1,1′-binaphthyl (BINAP, Strem, 59 mg, 0.094 mmol), and tert-BuONa (0.362 g, 3.8 mmol). This mixture was warmed to 85° C. and stirred for 18 h. At this point, the reaction was incomplete, so additional Pd₂(dba)₃ (43 mg, 0.047 mmol) and BINAP (59 mg, 0.094 mmol) were added and the mixture stirred for an additional 24 h. The reaction was cooled to ambient temperature, filtered through Celite® diatomaceous earth, concentrated under reduced pressure and purified via flash column chromatography (SiO₂, 50% hexanes-EtOAc) to give the title compound (40 mg, 0.113 mmol, 5% yield). MS (DCI/NH₃) m/z 355 (M+H)⁺.

### EXAMPLE 29C

### 2-(1-Phenyl-1H-pyrazol-4-yl)-octahydro-pyrrolo[3,4-c]pyrrole p-toluenesulfonate

To the product of Example 29B (40 mg, 0.113 mmol) in 4 mL CH₂Cl₂ was added 2 mL trifluoroacetic acid (TFA). This mixture stirred for 30 min at ambient temperature then was concentrated under reduced pressure. The residue was azeotroped twice with toluene to remove residual TFA and the crude product was purified via flash column chromatography (SiO₂, 1% NH₄OH:9% CH₃OH:90% CH₂Cl₂) to give 26 mg of the corresponding free amine (0.102 mmol, 91% yield). This material was dissolved in 5 mL 10% CH₃OH in EtOAc and 21 mg of p-toluenesulfonic acid in 2 mL 10% CH₃OH in EtOAc was added. Diethyl ether (−2 mL) was added to the resulting clear solution and, upon stirring at ambient temperature, a precipitate formed and was isolated via filtration (42 mg, 0.095 mmol, 93% yield). ¹H NMR (CH₃OH-d₄, 300 MHz) δ 2.35 (s, 3H), 2.98 (m, 2H), 3.18 (m, 2H), 3.23 (m, 2H), 3.35 (brd, J=9.5 Hz, 2H), 3.57 (m, 2H), 7.21 (m, 2H), 7.27 (ft, J=7.1, 1.4 Hz, 1H), 7.45 (m, 3H), 7.68 (m, 4H), 7.80 (d, J=0.7 Hz, 1H); MS (DCI/NH₃) m/z 255 (M+H)⁺; Anal. calculated for C₁₅H₁₈N₄.1.1C₇H₈O₃S: C, 61.44; H, 6.09; N, 12.63; Found: C, 61.04; H, 6.09; N, 12.45.

### EXAMPLE 30

### 2-(2-Methoxy-biphenyl-4-yl)-octahydro-pyrrolo[3,4-c]pyrrole

### EXAMPLE 30A

### 5-(3-Methoxy-phenyl)-hexahydro-pyrrolo[3,4-c]pyrrole-2-carboxylic Acid Tert-Butyl Ester

The product of Example 6C (1.0 g, 4.71 mmol), 3-bromoanisole (1.15 g, 6.12 mmol), tris(dibenzylideneacetone)dipalladium (0) (Pd₂(dba)₃, Strem, 86 mg, 0.094 mmol), racemic-2,2′-bis(diphenylphosphino)-1,1′-binaphthyl (BINAP, Strem, 0.117 g, 0.188 mmol), and tert-BuONa (0.724 g, 7.54 mmol) were combined in 20 mL toluene. This mixture was warmed to 85° C. and stirred for 18 h then was cooled to ambient temperature, filtered and concentrated under reduced pressure. The crude residue was purified via flash column chromatography (SiO₂, 50% hexanes-EtOAc) to give 1.45 g of the title compound (4.6 mmol, 97% yield). MS (DCI/NH₃) m/z 319 (M+H)⁺.

### EXAMPLE 30B

### 5-(4-Iodo-3-methoxy-phenyl)-hexahydro-pyrrolo[3,4-c]pyrrole-2-carboxylic Acid Tert-Butyl Ester

To the product of Example 30A (0.7 g, 2.2 mmol) in 30 mL CH₂Cl₂ at ambient temperature was added 1.16 g of TIOAc (Aldrich, 4.4 mmol) as described in Pirrung, M., et al, JACS, 2001, 123, 3638-3643. This mixture stirred for 5 min then 12 (0.67 g, 2.64 mmol) in 70 mL CH₂Cl₂ was added dropwise. Thallium (I) iodide formed a precipitate in the course of this reaction. This mixture stirred at ambient temperature for 2 h then was filtered. The filtrate was washed 1×15 mL 10% aqueous Na₂S₂O₃, 1×10 mL NaHCO₃, and 1×10 mL saturated, aqueous NaCl (brine). The organic material was concentrated under reduced pressure and purified via flash column chromatography (SiO₂, 50% hexanes-EtOAc) to give 0.68 g of the title compound (1.53 mmol, 70% yield). MS (DCI/NH₃) m/z 445 (M+H)⁺.

### EXAMPLE 30C

### 5-(2-Methoxy-biphenyl-4-yl)-hexahydro-pyrrolo[3,4-c]pyrrole-2-carboxylic Acid Tert-Butyl Ester

The product of Example 30B (0.68 g, 1.53 mmol), phenylboronic acid (Aldrich, 0.59 g, 3.07 mmol), tris(dibenzylideneacetone)dipalladium (0) (Pd₂(dba)₃, Strem, 56 mg, 0.061 mmol), 1,3-bis(2,6-di-1-propylphenyl)imidazolium chloride (Strem, 65 mg, 0.15 mmol), and 4 mL of 2M Na₂CO₃ in H₂O were combined in 20 mL PhCH₃. The mixture was warmed to 85° C. and stirred for 18 h; however, the mixture contained mostly starting material, so additional Pd₂(dba)₃ (56 mg, 0.061 mmol) and 1,3-bis(2,6-di-1-propylphenyl)imidazolium chloride (65 mg, 0.15 mmol) were added and the mixture stirred for another 18 h at 85° C. The reaction was cooled to ambient temperature, filtered, concentrated under reduced pressure and purified via flash column chromatography (SiO₂, 50% hexanes-EtOAc) to give 0.17 g of the title compound (0.43 mmol, 28% yield). MS (DCI/NH₃) m/z 395 (M+H)⁺.

### EXAMPLE 30D

### 2-(2-Methoxy-biphenyl-4-v)-octahydro-pyrrolo[3,4-c]pyrrole Trifluoroacetate

To the product of Example 30C (0.17 g, 0.43 mmol) in 6 mL CH₂Cl₂ was added 3 mL trifluoroacetic acid (TFA) as described in Example 11D to give 0.122 g of the title compound (0.30 mmol, 69% yield). ¹H NMR (CH₃OH-d₄, 300 MHz) δ 3.25 (m, 4H), 3.35 (m, 2H), 3.52 (m, 2H), 3.62 (m, 2H), 3.78 (s, 3H), 6.41 (dd, J=6.8, 2.4 Hz, 1H), 6.42 (s, 1H), 7.18 (m, 2H), 7.31 (m, 2H), 7.42 (m, 2H); MS (DCI/NH₃) m/z 295 (M+H)⁺; Anal. calculated for C₁₉H₂₂N₂O.CF₃CO₂H: C, 61.76; H, 5.68; N, 6.86; Found: C, 62.03; H, 5.91; N, 7.02.

### EXAMPLE 31

### 2-(2-Methoxy-biphenyl-4-yl)-5-methyl-octahydro-pyrrolo[3,4-c]pyrrole

### EXAMPLE 31A

### 2-(2-Methoxy-biphenyl-4-yl)-5-methyl-octahydro-pyrrolo[3,4-c]pyrrole

To the product of Example 30D (0.102 g, 0.25 mmol) in 3 mL 37% aqueous HCHO was added 54 mg NaBH(OAc)₃ (0.25 mmol). This material stirred at ambient temperature for 4 h then was quenched with 5 mL saturated, aqueous NaHCO₃. CH₂Cl₂ (5 mL) was added, the layers separated and the aqueous layer was extracted 3×5 mL CH₂Cl₂. The combined organics were dried over Na₂SO₄, concentrated under reduced pressure and purified via flash column chromatography (SiO₂, 1% NH₄OH:9% CH₃OH:90% CH₂Cl₂) to give 69 mg of the title compound (2.24 mmol, 90% yield) which was carried on to the next reaction without further purification.

### EXAMPLE 31B

### 2-(2-Methoxy-biphenyl-4-yl)-5-methyl-octahydro-pyrrolo[3,4-c]pyrrole p-toluenesulfonate

To the product of Example 31A (69 mg, 0.224 mmol) in 3 mL 10% EtOH in EtOAc was added p-toluenesulfonic acid (p-TsOH.H₂O, 45 mg, 0.24 mmol) in 2 mL 10% EtOH in EtOAc. Diethyl ether (1 mL) was added and the mixture stirred at ambient temperature until a precipitate formed. Filtration yielded 28 mg of the title compound (0.043 mmol, 19% yield). ¹H NMR (CH₃OH-d₄, 300 MHz) δ 2.35 (s, 6H), 2.92 and 2.98 (rotamer s, 3H), 3.19 (m, 3H) 3.38 (m, 3H), 3.63 (m, 3H), 3.77 (s, 3H), 3.98 (m, 1H), 6.50 (m, 1H), 6.52 (s, 1H), 7.16 (m, 1H), 7.22 (m, 5H), 7.32 (m, 2H), 7.42 (m, 2H), 7.70 (m, 4H); MS (DCI/NH₃) m/z 309 (M+H)⁺; Anal. calculated for C₂₀H₂₄N₂O.2C₇H₈O₃S: C, 62.55; H, 6.18; N, 4.29; Found: C, 62.17; H, 5.95; N, 4.18.

### EXAMPLE 32

### 2-Methyl-5-(3-phenyl-isoxazol-5-yl)-octahydro-pyrrolo[3,4-c]pyrrole

### EXAMPLE 32A

### 2-Benzyl-5-methyl-octahydro-pyrrolo[3,4-c]pyrrole

To 3.4 g of LiAlH₄ (90 mmol) in 100 mL diethyl ether at 0° C., was added 5-benzyl-2-methyl-tetrahydro-pyrrolo[3,4-c]pyrrole-1,3-dione (7.3 g, 30 mmol), which was prepared according to literature procedure (Torii, S.; et al. Chemistry Letters, 1996, 9, 747-748), in 50 mL diethyl ether. After the addition was complete, the reaction mixture was warmed to reflux and allowed to stir for 2 h. The mixture was then cooled to ambient temperature and was quenched by the sequential addition of 3.4 mL H₂O, 3.4 mL 15% aqueous NaOH, and 10.2 mL H₂O. The resulting slurry was filtered and the filtrate was dried over anhydrous MgSO₄ and concentrated under reduced pressure to give 5.2 g of the title compound (24 mmol, 80% yield). MS (DCI/NH₃) m/z 217 (M+H)⁺.

### EXAMPLE 32B

### 2-Methyl-octahydro-pyrrolo[3,4-c]pyrrole

The product of Example 32A (5.2 g, 24 mmol) and 2.6 g Pd(OH)₂/C (20%, wet) in 52 mL CH₃OH were placed under 60 psi of H₂ for 4 h at 50° C. The resulting mixture was cooled to ambient temperature, filtered and concentrated under reduced pressure to give 2.5 g of the title compound (19.8 mmol, 83% yield). MS (DCI/NH₃) m/z 127 (M+H)⁺.

### EXAMPLE 32C

### 2-Methyl-5-(3-phenyl-isoxazol-5-yl)-octahydro-pyrrolo[3,4-c]pyrrole

The product of Example 32B (1 g, 7.9 mmol), and 5-chloro-3-phenyl-isoxazole (1.4 g, 7.9 mmol) (prepared according to literature procedure: Dannhardt, G.; Obergrusberger, I. Chemiker-Zeitung 1989, 113, 109-113) in DBU (1.3 g, 8.6 mmol) were warmed to 140-145° C. for 40 min. The reaction mixture was cooled to ambient temperature, CH₂Cl₂ was added, and the crude material was purified by flash column chromatography (SiO₂, 10% CH₃OH—CH₂Cl₂ with 1% NH₄OH) to give 0.54 g of the title compound (2.0 mmol, 25% yield) which was carried on to the next step without further purification.

### EXAMPLE 32D

### 2-Methyl-5-(3-phenyl-isoxazol-5-yl)-octahydro-pyrrolo[3,4-c]pyrrole Fumarate

To the product of Example 32C (0.52 g, 1.93 mmol) in 7 mL 10% CH₃OH in diethyl ether was added fumaric acid (0.224 g, 1.93 mmol) in 5 mL 10% CH₃OH in diethyl ether. The resulting precipitate was isolated to give 0.65 g of the title compound (1.69 mmol, 87% yield). ¹H NMR (CH₃OH-d₄, 300 MHz) δ 2.84 (s, 3H), 3.15-3.32 (m, 3H), 3.42-3.62 (m, 7H), 5.62 (s, 1H), 7.42 (m, 3H), 7.65 (m, 2H), 8.11; MS (DCI/NH₃) m/z 270 (M+H)⁺; Anal. calculated for C₁₆H₁₉N₃O.C₄H₄O₄: C, 62.33; H, 6.01; N, 10.90; Found: C, 62.23; H, 5.93; N, 10.82.

### EXAMPLE 33

### (1S, 5S)-3-(6-Phenyl-pyridazin-3-yl)-3,6-diaza-bicyclo[3.2.0]heptane

### EXAMPLE 33A

### (2,2-Dimethoxy-ethyl)-carbamic Acid Benzyl Ester

Benzyl chloroformate (Aldrich, 231.3 g, 1.3 mol) was added gradually to a mixture of aminoacetaldehyde dimethyl acetal (Aldrich, 152.0 g, 1.3 mol) in toluene (750 mL) and aqueous NaOH (72.8 g, 1.82 mol; in 375 mL of water) at 10-20° C. After the addition was complete, the mixture was stirred at ambient temperature for 4 h. The layers were separated and the organic layer was washed with brine (2×100 mL) and concentrated under reduced pressure to provide the title compound as an oil (281.5 g, 90% yield). ¹H NMR (CDCl₄, 300 MHz) δ 3.33 (t, J=6.0 Hz, 2H), 3.39 (s, 6H), 4.37 (t, J=6.0 Hz, 1H), 5.11 (s, 2H), 7.30 (m, 5H); MS (DCI/NH₃) m/z 257 (M+NH₄)⁺, 240 (M+H)⁺.

### EXAMPLE 33B

### Allyl-(2,2-dimethoxy-ethyl)-carbamic Acid Benzyl Ester

The product of Example 33A (281.0 g, 1.18 mol) in dry toluene (1.0 L) was treated with powdered KOH (291.2 g, 5.20 mol) and triethylbenzylammonium chloride (Aldrich, 4.4 g, 0.02 mol). A solution of allyl bromide (Aldrich, 188.7 g, 1.56 mol) in toluene (300 mL) was then added dropwise over 1 hour at 20-30° C. The mixture was stirred for −18 h at ambient temperature and then water (300 mL) was added over 20 minutes at 20-30° C. The layers were separated and the aqueous phase was extracted with toluene (2×300 mL). The organic phases were combined, washed with brine (2×100 mL), dried (K₂CO₃), filtered and the filtrate concentrated under reduced pressure to provide the title compound as an oil (315.6 g, 1.13 mol, 96%, yield). ¹H NMR (MeOH-d₄, 300 MHz) δ 3.32 (s, 3H) 3.37 (m, 5H), 3.97 (d, J=5.4 Hz, 2H), 4.50-4.40 (m, 1H), 5.15 (m, 4H), 5.75 (m, 1H), 7.23 (m, 5H); MS (DCI/NH₃) m/z 297 (M+NH₄)⁺, 280 (M+H)⁺.

### EXAMPLE 33C

### Allyl-(2-oxo-ethyl)-carbamic Acid Benzyl Ester

The product of Example 33B (314.0 g, 1.125 mol) was treated with formic acid (88%, 350 mL) at room temperature and allowed to stir for 15 hours. Most of the formic acid was removed by concentration under reduced pressure at 40-50° C. The residue was extracted with ethyl acetate (3×500 mL). The extracts were combined and washed with brine until the wash had a pH=6-7. The organic phase was concentrated under reduced pressure to provide the title compound as a slightly yellow oil (260.0 g, 1.12 mmol 99% yield). ¹H NMR (CDCl₃, 300 MHz) δ 3.20 (m, 1H), 3.97 (m, 2H), 4.10 (m, 1H), 5.10 (m, 4H), 5.75 (m, 1H), 7.45 (m, 5H), 9.50 (d, J=6.4 Hz, 1H); MS (DCI/NH₃) m/z 234 (M+H)⁺.

### EXAMPLE 33D

Allyl-(2-hydroxyimino-ethyl)-carbamic Acid Benzyl Ester

The product of Example 33C (260 g, 1.12 mol) in acetonitrile (1.5 L) was treated with sodium acetate trihydrate (170.6 g, 4.41 mol, in 0.75L distilled water) and NH₂OH.hydrochloride (98.0 g, 4.41 mol) under N₂. The mixture was stirred at ambient temperature over 20 hours. The volatiles were removed under reduced pressure and the residue was extracted with ethyl acetate (2×750 mL). The combined organic phases were washed with brine until the wash had a pH=7. The organic phase was concentrated under reduced pressure to provide the title compound as an oil (271 g, 1.09 mol, 98% yield). ¹H NMR (MeOH-d₄, 300 MHz) δ 3.94 (m, 2H), 3.98 (d, J=5.5 Hz, 1H), 4.17 (d, J=4.4 Hz, 1H), 5.30 (m, 4H), 5.60 (m, 1H), 7.40 (m, 5H); MS (DCI/NH₃) m/z 266 (M+NH₄)⁺, 249 (M+H)⁺.

### EXAMPLE 33E

### Benzyl(cis)-3-amino-4-(hydroxymethyl)-1-pyrrolidinecarboxylate

A solution of the product of Example 33D (240 g, 0.97 mol) in xylene (1.0 L) was heated at reflux under N₂ for 10 hours. The resulting brown solution was cooled to 10-15° C. and acetic acid (1.0 L) was added under N₂. Zinc powder (100 g, 1.54 mol) was added gradually, and the gray mixture was stirred at ambient temperature for 3 hours. The mixture was filtered and water (1.0 L) was added to the filtrate. The filtrate was stirred for 10 minutes and the brown organic layer was separated. The aqueous phase was washed well with xylenes (4×400 mL) and then concentrated under reduced pressure to a volume of approximately 200 mL. This residue was adjusted to pH 9-10 by cautious addition of saturated, aqueous Na₂CO₃. The precipitated white solid was removed by filtration and the filtrate was extracted with CHCl₃ (3×600 mL). The combined organic phases were washed with saturated, aqueous Na₂CO₃ solution (2×50 mL) and dried over anhydrous Na₂CO₃. The mixture was filtered through a short column of diatomaceous earth and the filtrate was concentrated under reduced pressure to provide the title compound as a slightly yellow oil (145 g, 0.58 mol, 60% yield). ¹H NMR (MeOH-d₄, 300 MHz) δ 2.40 (m, 1H), 3.30 (m, 2H), 3.80-3.50 (m, 5H), 5.10 (s, 2H), 7.35 (m, 5H); MS (DCI/NH₃) m/z 251 (M+H)⁺.

### EXAMPLE 33F

### Benzyl(cis)-2,2-dimethylhexahydropyrrolo[3,4-d][1,3]oxazine-6(4H)-carboxylate (R)-Mandelate

The product of Example 33E (140 g, 0.56 mol) in dry acetone (150 mL) was treated with 2-methoxypropene (55 mL, 0.57 mol) at ambient temperature for ˜18 h. The reaction mixture was concentrated under reduced pressure and the residue was dissolved in dry acetone (750 mL). (R)-Mandelic acid (85 g, 0.56 mol) was added and the brown solution was stirred at ambient temperature for 48 hours. The precipitate was isolated by filtration and dried under reduced pressure to a mixture of the title compound as a white solid (57.0 g, 0.13 mol, yield, 23%) and the hydrolyzed compound benzyl(cis)-3-amino-4-(hydroxymethyl)-1-pyrrolidinecarboxylate (R)-mandelate. ¹H NMR for title compound (MeOH-D₄, 300 MHz) δ 1.20-1.40 (m, 3H), 2.09 (s, 3H), 3.30 (m, 1H), 3.48-3.75 (m, 6H), 4.20 (m, 1H), 5.10 (m, 3H), 7.25-7.52 (m, 10H); MS (DCI/NH₃) m/z 291 (M+H)⁺ (for the title compound) 251 (M+H)⁺ (for the hydrolyzed product).

### EXAMPLE 33G

### Benzyl(3S,4S)-3-[(tert-butoxycarbonyl)amino]-4-(hydroxymethyl)-1-pyrrolidinecarboxylate

The product of Example 33F (56 g, 127 mmol) in ethanol (50 mL) was treated with 5% aqueous H₂SO₄ (100 mL) at ambient temperature and allowed to stir for 16 hours. The mixture was adjusted to pH ˜10 with 20% aqueous NaOH (50 mL) and then the mixture was treated with di-t-butyl dicarbonate (41.5 g, 190 mmol) in ethanol (50 mL) at 10-20° C. After stirring at ambient temperature for 4 hours, the ethanol was removed under reduced pressure and the residue was extracted with ethyl acetate (3×500 mL). The combined organic phases were washed with brine (2×100 mL) and concentrated under reduced pressure to provide the title compound (43.7 g, 0.125 mol, 98% yield). ¹H NMR (MeOH-d₄, 300 MHz) δ 1.46 (s, 9H), 2.50 (m, 1H), 3.25 (m, 1H), 3.40 (m, 1H), 3.50-3.75 (m, 4H), 4.20 (m, 1H), 5.10 (s, 2H), 7.35 (m, 5H); MS (DCI/NH₃) m/z 368 (M+NH₄)⁺, 351 (M+H)⁺. The enantiopurity of the title compound was determined to be ≧99% ee by HPLC (HPLC conditions: Chiracel AD column; ethanol/hexanes=20/80, flow rate, 1.0 mL/min; uv 220 nm; retention time for the title compound as the more mobile isomer: 10.8 minutes; Retention time for less mobile isomer: 13.9 minutes; reference: JP 2000 026408).

### EXAMPLE 33H

### Benzyl(3S,4S)-3-[(tert-butoxycarbonyl)amino]-4-{[(methylsulfonyl)oxy]methyl}-1-pyrrolidinecarboxylate

The product of Example 33G (43.7 g, 125 mmol) and triethylamine (25.2 g, 250 mmol) in CH₂Cl₂ (600 mL) were treated with methanesulfonyl chloride (12.6 mL, 163 mmol) over 30 minutes at −10° C. The solution was allowed to warm to ambient temperature over 1 hour and was monitored by HPLC. When the reaction was completed, it was quenched with water (100 mL). The layers were separated and the aqueous phase was extracted with CH₂Cl₂ (2×400 mL). The combined organic phases were washed with brine (2×100 mL), dried over Na₂SO₄, filtered and the filtrate concentrated under reduced pressure to provide the title compound as a brown oil (52.0 g, 0.12 mol, 97% yield). ¹H NMR (CDCl₃, 300 MHz) δ 1.46 (s, 9H), 2.80 (m, 1H), 3.08 (s, 3H), 3.40(m, 2H), 3.70 (m, 2H), 4.10 (m, 1H), 4.40 (m, 2H), 4.75 (m, 1H), 5.16 (s, 2H), 7.30 m, 5H); MS (DCI/NH₃) m/z 446 (M+NH₄)⁺, 429 (M+H)⁺. HPLC conditions: HPLC conditions: Zorbax-XDB-C8 column 4.6×250 mm with solvents H₂O (0.2v.% HClO₄)/MeCN (from v.80:20 to 10:90 within 15 min.) at 1.0 mL/Min., UV detection @220 nm. 20/80, flow rate, 1.0 mL/min; uv 220 nm; t_{R}=13.1 minutes.

### EXAMPLE 331

### Benzyl(3S,4S)-3-(amino)-4-{[(methylsulfonyl)oxy]methyl}-1-pyrrolidinecarboxylate Trifluroacetate

The product of Example 33H (43.7 g, 125 mmol) in CH₂Cl₂ (150 mL) was treated with trifluoroacetic acid (50 mL) at ambient temperature and allowed to stir for 1 h. The reaction was monitored with HPLC. After the reaction went to completion, the mixture was concentrated under reduced pressure to give the title compound in quantitative yield. ¹H NMR (CDCl₃, 300 MHz) δ 2.80 (m, 1H), 3.15 (s, 3H), 3.40 (m, 1H), 3.70 (m, 3H), 4.10 (m, 1H), 4.05 (m, 1H), 4.44 (m, 2H), 5.16 (s, 2H), 7.30-7.50 (m, 5H); MS (DCI/NH₃) m/z 329 (M+H—CF₃CO₂H)⁺. HPLC conditions: Zorbax-XDB-C8 column 4.6×250 mm with solvents H₂O (0.2v.% HClO₄)/CH₃CN (from v.80:20 to 10:90 within 15 min.) at 1.0 mL/Min., UV detection @220 nm. 20/80, flow rate, 1.0 mL/min; uv 220 nm; t_{R}=8.2 minutes.

### EXAMPLE 33J

### Benzyl(1S,5S)-3,6-diazabicyclo[3.2.0]heptane-3-carboxylate

The product of Example 331 (125 mmol) was dissolved in ethanol (250 mL) and adjusted to pH ˜12 with 25% aqueous NaOH. The mixture was warmed to 60° C. for 1.5 h and monitored via HPLC. After the reaction went to completion, it was allowed to cool down to ambient temperature and used for the next step with the exception of ˜1 mL which was used for characterization. The ˜1 mL sample was concentrated under reduced pressure to remove most of the ethanol. The residue was extracted with CHCl₃ (2×5 mL). The extracts were combined, washed with brine (3×2 mL) and then passed through a short column of diatomaceous earth. The filtrate was concentrated under reduced pressure to provide the title compound as a yellow oil. ¹H NMR (MeOH-d₄, 300 MHz) δ 3.30-3.16 (m, 3H), 3.36 (m, 1H), 3.82 (m, 3H), 4.55 (m, 1H), 5.20 (s, 2H), 7.36 (m, 5H); MS (DCI/NH₃) m/z 250 (M+NH₄)⁺, 233 (M+H)⁺. HPLC conditions: Zorbax-XDB-C8 column 4.6×250 mm with solvents H₂O (0.2v.% HClO₄)/MeCN (from v.80:20 to 10:90 within 15 min.) at 1.0 mL/Min., UV detection @220 nm. 20/80, flow rate, 1.0 mL/min; uv 220 nm; t_{R}=7.2 min.

### EXAMPLE 33K

### 3-Benzyl, 6-tert-butyl-(1R,5S)-3,6-diazabicyclo[3.2.0]heptane-3,6-dicarboxylate

To the solution of Example 33J (˜125 mmol) was slowly added di-t-butyl dicarbonate (40.9 g, 188 mmol) ethanol (50 mL) solution over 30 min at ambient temperature. The mixture was stirred at ambient temperature for an additional 0.5-1 h with monitoring by HPLC. After the reaction went to completion, it was concentrated under reduced pressure to remove most of the ethanol. The residue was extracted with EtOAc (3×500 mL). The extracts were combined, washed with brine (3×50 mL) and stirred with KHSO₄ (5%, 100 mL) for 10 min. to remove unreacted di-t-butyl dicarbonate. The layers were separated and the organic layer was washed with brine (3×50 mL) and passed through a short column of diatomaceous earth. The filtrate was concentrated under reduced pressure to provide the title compound as a yellow oil (40.2 g, 97% three-step yield). ¹H NMR (MeOH-d₄, 300 MHz) δ 1.4(s, 9H), 3.10(m, 2H), 3.30 (m, 1H), 3.45 (m, 1H), 3.90 (d, J=12.2 Hz, 1H), 4.06 (m, 2H), 4.66 (dd, J=6.4, 2.0 Hz, 1H), 5.16 (s, 2H), 7.36 (m, 5H); MS (DCI/NH₃) m/z 333 (M+H)⁺. HPLC conditions: Zorbax-XDB-C8 column 4.6×250 mm with solvents H₂O (0.2v.% HClO₄)/MeCN (from v.80:20 to 10:90 within 15 min.) at 1.0 mL/Min., UV detection @220 nm. 20/80, flow rate, 1.0 mL/min; uv 220 nm; t_{R}=13.6 minutes.

### EXAMPLE 33L

### tert-Butyl(1R,5S)-3,6-diazabicyclo[3.2.0]heptane-6-carboxylate

The product of Example 33K (40.0 g, 0.120 mol) was dissolved in methanol (400 mL) and treated with Pd/C (10 wt %, 4.0 g) under H₂ at ambient temperature for 10 h. The reaction was monitored with HPLC. After the reaction was complete, the catalyst was removed by filtration through a short column of diatomaceous earth. The filtrate was concentrated under reduced pressure to provide the title compound as a colorless oil (22.8 g, 11.5 mmol, 96% yield). ¹H NMR (MeOH-d₄, 300 MHz) δ 1.43 (s, 9H), 2.47 (dd, J=12.6, 3.8 Hz, 1H), 2.62 (dd, J=12.2, 5.7 Hz, 1H), 2.96 (m, 1H), 3.05 (d, J=12.2 Hz, 1H), 3.22 (d, J=12.5 Hz, 1H), 3.45 (m, 1H), 3.95 (m, 1H), 4.63 (dd, J=6.1, 3.7 Hz, 1H); MS (DCI/NH₃) m/z 199 (M+H)⁺. HPLC conditions: Zorbax-XDB-C8 column 4.6×250 mm with solvents H₂O (0.2v.% HClO₄)/MeCN (from v.80:20 to 10:90 within 15 min.) at 1.0 mL/Min., UV detection @ 220 nm. 20/80, flow rate, 1.0 mL/min; uv 220 nm; t_{R}=8.6 minutes.

### EXAMPLE 33M

### (1R, 5S)-3-(6-Phenyl-pyridazin-3-yl)-3,6-diaza-bicyclo[3.2.0]heptane-6-carboxylic Acid Tert-Butyl Ester

The product of Example 33L (0.2 g, 1 mmol) was coupled with 3-chloro-6-phenyl-pyridazine (Aldrich, 0.29 g, 1.5 mmol) catalyzed by Pd₂(dba)₃ (Strem, 18 mg, 0.02 mmol) and 1,3-bis(2,6-di-1-propylphenyl)imidazolium chloride (26 mg, 0.06 mmol) with Cs₂CO₃ (330 mg, 1 mmol) in toluene (dry, 10 mL) at 110° C. under N₂ over 70 hours. After the reaction went to completion, the mixture was cooled down to ambient temperature and quenched with 5 mL of water. The layers were separated and the aqueous layer was extracted with EtOAc (3×10 mL). The extracts were combined and concentrated under reduced pressure. The residue was purified by chromatography (SiO₂, 50% EtOAc: hexane; R_{f}. 0.40) giving the title compound as a brown oil (150 mg, 0.42 mmol, 43% yield). ¹H NMR (MeOH-d₄, 300 MHz) δ 1.45 (s, 9H), 3.26 (m, 2H), 3.36 (m, 1H), 3.61 (m, 1H), 4.10 (m, 2H), 4.30 (m, 1H), 4.89 (m, 1H), 7.24 (d, J=8.5 Hz, 1H), 7.40-7.53 (m, 3H), 7.90 (d, J=9.4 Hz, 1H), 7.92-7.80 (m, 2H); MS (DCI/NH₃) m/z 353 (M+H)⁺.

### EXAMPLE 33N

### (1S, 5S)-3-(6-Phenyl-pyridazin-3-yl)-3,6-diaza-bicyclo[3.2.0]heptane p-toluenesulfonate

The product of Example 33M (150 mg, 0.42 mmol) was treated with TsOH.H₂O (162 mg, 0.86 mmol) at reflux for 2 h. The title compound was obtained as a solid by filtration (230 mg, 0.39 mmol, 90% yield). ¹H NMR (MeOH-d₄, 300 MHz) δ 2.24 (s, 6H), 3.70 (m, 2H), 3.81 (dd, J=13.9, 5.7 Hz, 1H), 3.88 (dd, J=13.2, 4.0 Hz, 1H), 4.32 (m, 2H), 4.60 (d, J=13.9 Hz, 1H), 5.22 (m, 1H), 7.19 (d, J=7.8 Hz, 4H), 7.62 (m, 3H), 7.67 (d, J=8 Hz, 4H), 7.90-8.02 (m, 3H), 8.40 (d, J=9.5 Hz, 1H); MS (DCI/NH₃) m/z 253 (M+H)⁺; Anal. calculated for C₁₅H₁₆N₄.2C₇H₈SO₃: C, 58.37; H, 5.41; N, 9.39. Found: C, 58.27; H, 5.29; N, 9.21.

### EXAMPLE 34

### (1S, 5S)-6-Methyl-3-(6-phenyl-pyridazin-3-yl)-3,6-diaza-bicyclo[3.2.0]heptane

### EXAMPLE 34A

### (1S, 5S)-6-Methyl-3-(6-phenyl-pyridazin-3-yl)-3,6-diaza-bicyclo[3.2.0]heptane

The product of Example 33M (100 mg, 0.17 mmol) was treated with formalin (30%, 1 mL) and NaBH(OAc)₃ (0.23 g, 1 mmol) in CH₃CN (5 mL) at ambient temperature for ˜18 h. The mixture was quenched with saturated, aqueous Na₂CO₃ (5 mL) and extracted with CHCl₃/I-PrOH (10:1) (3×10 mL). The extracts were combined and concentrated under reduced pressure. The residue was purified by chromatography (SiO₂, 90:10:2 CH₂Cl₂: MeOH:NH₄OH, R_{f}. 0.20) giving the title compound as a brown oil (40 mg, 0.15 mmol, 88% yield). ¹H NMR (MeOH-d₄, 300 MHz) δ 2.45 (s, 3H), 3.30-3.44 (m, 3H), 3.60 (dd, J=11.2, 8.5 Hz, 1H), 4.00 (m, 2H), 4.10 (m, 1H), 4.85 (m, 1H), 7.17 (d, J=9.5 Hz, 1H), 7.39-7.51 (m, 3H), 7.88 (d, J=9.5 Hz, 1H), 7.90-8.02 (m, 2H); MS (DCI/NH₃) m/z 267(M+H)⁺.

### EXAMPLE 34B

### (1S, 5S)-6-Methyl-3-(6-phenyl-pyridazin-3-yl)-3,6-diaza-bicyclo[3.2.0]heptane Fumarate

The product of Example 34A (40 mg, 0.15 mmol) was treated with fumaric acid (21 mg, 0.18 mmol) in i-PrOH (5 mL) for ˜18 h. The title compound was obtained as a white solid (51 mg, 0.12 mmol, 82% yield). ¹H NMR (MeOH-d₄, 300 MHz) δ 2.88 (s, 3H), 3.37-3.60 (m, 3H), 3.89 (dd, J=11.2, 4.7 Hz, 1H), 4.00-4.15 (m, 2H), 4.48 (d, J=13.6 Hz, 1H), 4.90 (m, 1H), 6.69 (s, 2.4H), 7.32 (d, J=9.5 Hz, 1H), 7.39-7.53 (m, 3H), 7.90-8.02 (m, 3H); MS (DCI/NH₃) m/z 267 (M+H)⁺; Anal. calculated for C₁₆H₁₈N₄.1.2C₄H₄O₄.0.2H₂O: C, 61.05; H, 5.71; N, 13.69. Found: C, 60.84; H, 5.38; N, 13.93.

### EXAMPLE 35

### (1R, 5S)-6-(6-Phenyl-pyridazin-3-yl)-3,6-diaza-bicyclo[3.2.0]heptane

### EXAMPLE 35A

### (1S, 5S)-6-(6-Phenyl-pyridazin-3-yl)-3,6-diaza-bicyclo[3.2.0]heptane-3-carboxylic Acid Benzyl Ester

The product of 33J (230 mg, 1 mmol) was coupled with 3-chloro-6-phenyl-pyridazine (Aldrich, 280 mg, 1.5 mmol) as described in Example 33M. The title compound was obtained as an oil (340 mg, 0.88 mmol, 88% yield). ¹H NMR (MeOH-d₄, 300 MHz) δ 3.26 (dd, J=12.8, 4.0 Hz, 1H), 3.31-3.45 (m, 2H), 3.77 (dd, J=8.1, 3.4 Hz, 1H), 4.01 (d, J=11.1 Hz, 1H), 4.20-4.30 (m, 2H), 4.99 (dd, J=6.1, 4.5 Hz, 1H), 5.00-5.22 (m, 2H), 6.88 (d, J=9.5 Hz, 1H), 7.10-7.50 (m, 8H), 7.83 (d, J=9.5 Hz, 1H), 7.88-7.98 (m, 2H); m/z 387 (M+H)⁺.

### EXAMPLE 35B

### (1S, 5S)-6-(6-Phenyl-pyridazin-3-yl)-3,6-diaza-bicyclo[3.2.0]heptane

The product of Example 35A (340 mg, 0.88 mmol) was treated with Pd/C (10 wt %, 100 mg) in EtOH (200 proof, 20 mL) at ambient temperature under 1 atm. of H₂ for ˜18 h. The catalyst was filtered off and the filtrate was concentrated under reduced pressure to give the title compound as a solid (180 mg, 81% yield): ¹H NMR (MeOH-d₄, 300 MHz) δ 2.61 (dd, J=12.8, 3.8 Hz, 1H), 2.76 (dd, J=12.5, 6.0 Hz, 1H), 3.19 (d, J=12.5 Hz, 1H), 3.24 (m, 1H), 3.41 (d, J=13.1 Hz, 1H), 3.77 (dd, J=8.7, 4.0 Hz, 1H), 4.20 (t, J=8.1 Hz, 1H), 4.96 (dd, J=6.2, 2.5 Hz, 1H), 6.88 (d, J=9.4 Hz, 1H), 7.38-7.49 (m, 3H), 7.82 (d, J=9.4 Hz, 1H), 7.90 (m, 2H); MS (DCI/NH₃) m/z 253 (M+H)⁺.

### EXAMPLE 35C

### (1 S 5S)-6-(6-Phenyl-pyridazin-3-yl)-3,6-diaza-bicyclo[3.2.0]heptane Fumarate

The product of Example 35B (50 mg, 0.2 mmol) was treated with fumaric acid (25 mg, 0.22 mmol) in EtOAc/MeOH (10/1) (5 mL) at ambient temperature for 18 h. The title compound was obtained as a solid (60 mg, 0.16 mmol, 80% yield): ¹H NMR (MeOH-d₄, 300 MHz) 63.25 (dd, J=13.2, 4.0 Hz, 1H), 2.38 (dd, J=12.2, 7.0 Hz, 1H), 3.50 (m, 1H), 3.74 (d, J=12.3 Hz, 1H), 3.87 (d, J=12.9 Hz, 1H), 3.90 (dd, J=8.6, 3.4 Hz, 1H), 4.29 (t, J=8.3 Hz, 1H), 5.21 (dd, J=6.4, 3.6 Hz, 1H), 6.67 (s, 2H), 6.99 (d, J=9.5 Hz, 1H), 7.38-7.52 (m, 3H), 7.90-7.99 (m, 3H); MS (DCI/NH₃) m/z 253 (M+H)⁺; Anal. calculated for C₁₅H₁₆N₄.C₄H₄O₄.0.3H₂O: C, 61.05; H, 5.55; N, 14.99. Found: C, 61.27; H, 5.55; N, 14.63.

### EXAMPLE 36

### (1R, 5S)-3-Methyl-6-(6-phenyl-pyridazin-3-yl)-3,6-diaza-bicyclo[3.2.0]heptane

### EXAMPLE 36A

### (1R, 5S)-3-Methyl-6-(6-phenyl-pyridazin-3-yl)-3,6-diaza-bicyclo[3.2.0]heptane

The product of Example 35B (0.28 mmol) was coupled with formalin following the procedure of Example 34A. The title compound was obtained as a solid (50 mg, 0.19 mmol, 67% yield). ¹H NMR (MeOH-d₄, 300 MHz) δ 2.32 (dd, J=12.6, 4.1 Hz, 1H), 2.41 (dd, J=10.9, 6.4 Hz, 1H), 2.55 (s, 3H), 3.26-3.35 (m, 2H), 3.55 (d, J=10.6 Hz, 1H), 3.89 (dd, J=8.4, 4.0 Hz, 1H), 4.22 (t, J=8.1 Hz, 1H), 5.00(dd, J=6.8, 2.7 Hz, 1H), 6.88 (d, J=9.2 Hz, 1H), 7.38-7.50 (m, 3H), 7.84 (d, J=9.5 Hz, 1H), 7.93 (m, 2H); MS (DCI/NH₃) m/z 267 (M+H)⁺.

### EXAMPLE 36B

### (1R, 5S)-3-Methyl-6-(6-phenyl-pyridazin-3-yl)-3,6-diaza-bicyclo[3.2.0]heptane Fumarate

The product of Example 36A (50 mg, 0.19 mmol) was treated with fumaric acid (25 mg, 0.22 mmol) in EtOAc/MeOH (10/1) (5 mL) at ambient temperature for ˜18 h. The title compound was obtained as a solid (86 mg, 0.19 mmol, 100% yield). ¹H NMR (MeOH-d₄, 300 MHz) δ 3.00 (s, 3H), 3.12 (dd, J=12.2, 3.7 Hz, 1H), 3.20 (dd, J=12.2, 7.5 Hz, 1H), 3.50 (m, 1H), 3.89 (d, J=12.2 Hz, 1H), 3.93 (dd, J=8.4, 3.4 Hz, 1H), 4.01 (d, J=12.2 Hz, 1H), 4.29 (t, J=8.2 Hz, 1H), 5.21 (dd, J=7.1, 3.7 Hz, 1H), 6.69 (s, 3H), 7.01 (d, J=9.2 Hz, 1H), 7.38-7.52 (m, 3H), 7.90-7.99 (m, 3H); MS (DCI/NH₃) m/z 267 (M+H)⁺; Anal. calculated for C₁₆H₁₈N₄.1.5C₄H₄O₄.0.5H₂O: C, 58.79; H, 5.61; N, 12.47. Found: C, 58.86; H, 5.21; N, 12.25.

### EXAMPLE 37

### (1R, 5R)-3-(6-Phenyl-pyridazin-3-yl)-3,6-diaza-bicyclo[3.2.0]heptane

### EXAMPLE 37A

### Benzyl(cis)-2,2-dimethylhexahydropyrrolo[3,4-d][1,3]oxazine-6(4H)-carboxylate (S)-Mandelate

The product of Example 33E was treated with (S)-mandelic acid following the procedure of Example 33F to provide the title compound.

### EXAMPLE 37B

### Benzyl(3R,4R)-3-[(tert-butoxycarbonyl)amino]-4-(hydroxymethyl)-1-pyrrolidinecarboxylate

The product of Example 37A was treated with 5% aqueous sulfuric acid and di-tert-butyl dicarbonate following the procedure of Example 33G to provide the title compound.

### EXAMPLE 37C

### Benzyl(3R,4R)-3-[(tert-butoxycarbonyl)amino]-4-{[(methylsulfonyl)oxy]methyl}-1-pyrrolidinecarboxylate

The product of Example 37B was treated with methanesulfonyl chloride and triethylamine following the procedure of Example 33H to provide the title compound.

### EXAMPLE 37D

### Benzyl(3R,4R)-3-(amino)-4-{[(methylsulfonyl)oxy]methyl-}-1-pyrrolidinecarboxylate Trifluroacetate

The product of Example 37C was treated with trifluoroacetic acid following the procedure of Example 331 to provide the title compound.

### EXAMPLE 37E

### Benzyl(1R,5R)-3,6-diazabicyclo[3.2.0]heptane-3-carboxylate

The product of Example 37D was treated with 25% aqueous sodium hydroxide following the procedure of Example 33J to provide the title compound. ¹H NMR (MeOH-d₄, 300 MHz) δ 3.30-3.16 (m, 3H), 3.36 (m, 1H), 3.82 (m, 3H), 4.55 (m, 1H), 5.20 (s, 2H), 7.36 (m, 5H); MS (DCI/NH₃) m/z 250 (M+NH₄)⁺, 233 (M+H)⁺.

### EXAMPLE 37F

### 3-Benzyl, 6-tert-butyl-(1S,5R)-3,6-diazabicyclo[3.2.0]heptane-3,6-dicarboxylate

The product of Example 37E was treated with di-tert-butyl dicarbonate following the procedure of Example 33K to provide the title compound.

### EXAMPLE 37G

### Tert-Butyl(1S,5R)-3,6-diazabicyclo[3.2.0]heptane-6-carboxylate

The product of Example 37F was treated with Pd/C under a hydrogen atmosphere following the procedure of Example 33L to provide the title compound. ¹H NMR (MeOH-d₄, 300 MHz) 1.43 (s, 9H), 2.47 (dd, J=12.6, 3.8 Hz, 1H), 2.62 (dd, J=12.2, 5.7 Hz, 1H), 2.96 (m, 1H), 3.05 (d, J=12.2 Hz, 1H), 3.22 (d, J=12.5 Hz, 1H), 3.45 (m, 1H), 3.95 (m, 1H), 4.63 (dd, J=6.1, 3.7 Hz, 1H); MS (DCI/NH₃) m/z 199 (M+H)⁺.

### EXAMPLE 37H

### (1S, 5R)-3-(6-Phenyl-pyridazin-3-yl)-3,6-diaza-bicyclo[3.2.0]heptane-6-carboxylic Acid Tert-Butyl Ester

The product of Example 37G (200 mg, 1 mmol) was coupled with 3-chloro-6-phenyl-pyridazine following the procedure described in Example 33M. ¹H NMR (MeOH-d₄, 300 MHz) δ 1.45 (s, 9H), 3.26 (m, 2H), 3.36 (m, 1H), 3.61 (m, 1H), 4.10 (m, 2H), 4.30 (m, 1H), 4.89 (m, 1H), 7.22 (d, J=8.5 Hz, 1H), 7.40-7.53 (m, 3H), 7.90 (d, J=9.4 Hz, 1H), 7.92-7.80 (m, 2H); MS (DCI/NH₃) m/z 353 (M+H)⁺.

### EXAMPLE 37I

### (1R, 5R) 3-(6-Phenyl-pyridazin-3-yl)-3,6-diaza-bicyclo[3.2.0]heptane p-toluenesulfonate

The product of Example 37H (180 mg, 0.5 mmol) was treated with p-toluenesulfonic acid monohydrate (114 mg, 0.6 mmol) in EtOAc (10 mL) at 80° C. over 2h. The title compound was obtained as a solid (190 mg, 0.75 mmol, 90% yield). ¹H NMR (MeOH-d₄, 300 MHz) δ 2.23 (s, 3H), 3.34 (m, 1H), 3.70 (m, 1H), 3.81 (dd, J=13.9, 3.8 Hz, 1H), 3.86 (m, 1H), 4.32 (m, 2H), 4.58 (d, J=13.6 Hz, 1H), 5.22 (m, 1H), 7.20 (d, J=8.1 Hz, 2H), 7.62 (m, 3H), 7.67 (d, J=8.5 Hz, 2H), 7.80 (m, 3H), 8.42 (d, J=9.9 Hz, 1H); MS (DCI/NH₃) m/z 253 (M+H)⁺.

### EXAMPLE 38

### (1R, 5R)-6-Methyl-3-(6-phenyl-pyridazin-3-yl)-3,6-diaza-bicyclo[3.2.0]heptane

### EXAMPLE 38A

### (1R, 5R)-6-Methyl-3-(6-phenyl-pyridazin-3-yl)-3,6-diaza-bicyclo[3.2.0]heptane

The product of Example 371 was treated with formalin following the procedure of Example 34A to provide the title compound.

### EXAMPLE 38B

### (1R, 5R)-6-Methyl-3-(6-phenyl-pyridazin-3-yl)-3,6-diaza-bicyclo[3.2.0]heptane p-toluenesulfonate

The product of Example 38A was treated with p-toluenesulfonic acid monohydrate to provide the title compound. ¹H NMR (MeOH-d₄, 300 MHz) δ 2.36 (s, 3H), 2.54 (s, 3.6H), 3.32 (m, 2H), 3.40-3.60 (m, 3H), 4.02 (dd, J=11.4, 2.2 Hz, 1H), 4.12 (d, J=12.3 Hz, 1H), 4.28 (m, 1H), 7.15-7.25 (m, 3H), 7.35-7.60 (m, 3.4H), 7.70 (d, J=8.3 Hz, 1H), 7.90-8.00 (m, 4.4H); MS (DCI/NH₃) m/z 267 (M+H)⁺; Anal. calculated for C₁₆H₁₈N₄.1.2C₇H₈SO₃.0.5H₂O: C, 60.80; H, 5.98; N, 11.62. Found: C, 61.07; H, 6.29; N, 11.52.

### EXAMPLE 39

### (1R, 5R)-3-(6-Benzo[1,3]dioxol-5-yl-pyridazin-3-yl)-3,6-diaza-bicyclo[3.2.0]heptane

### EXAMPLE 39A

### (1S, 5R)-3-(6-Chloro-pyridazin-3-yl)-3,6-diaza-bicyclo[3.2.0]heptane-6-carboxylic Acid Tert-Butyl Ester

The product of Example 37G (2.0 g, 10 mmol) was coupled with 3,6-dichloropyridazine (Aldrich, 2.23 g, 15 mmol) following the procedure of Example 33M. The title compound was obtained as an oil (2.4 g, 7.69 mmol, 77% yield). ¹H NMR (MeOH-d₄, 300 MHz) δ 1.45 (s, 9H), 3.19 (dd, J=11.8, 4.4 Hz, 1H), 3.23-3.34 (m, 2H), 3.58 (m, 1H), 3.99-4.20 (m, 3H), 4.87 (m, 1H), 7.18 (d, J=9.5 Hz, 1H), 7.45 (dd, J=9.5 Hz, 1H); MS (DCI/NH₃) m/z 311 (M+H)⁺, 313 (M+H)⁺.

### EXAMPLE 39B

### (1S, 5R)-3-(6-Benzo[1,3]dioxol-5-yl-pyridazin-3-yl)-3,6-diaza-bicyclo[3.2.0]heptane-6-carboxylic Acid Tert-Butyl Ester

The product of Example 39A (200 mg, 0.64 mmol) was coupled with benzo[1,3]dioxole-5-boronic acid (166 mg, 1 mmol) catalyzed by Pd₂(dba)₃ (Strem, 18 mg, 0.02 mmol) and 1,3-bis(2,6-di-1-propylphenyl)imidazolium chloride (26 mg, 0.06 mmol) with Na₂CO₃ (2M, 2 mL, 4 mmol) in toluene (8 mL) at 110° C. under N₂ over 15 hours. After the reaction went to completion, the mixture was cooled down to ambient temperature and was diluted with 50 mL EtOAc. The solution was washed with water (2×5 mL). The organic layer was concentrated under reduced pressure and the residue was purified by column chromatography (SiO₂, 50% EtOAc-hexane; R_{f}. 0.40) giving the title compound as a brown oil (220 mg, 0.55 mmol, 88% yield). ¹H NMR (MeOH-d₄, 300 MHz) δ 1.45 (s, 9H), 3.21 (dd, J=11.8, 4.4 Hz, 1H), 3.33 (m, 1H), 3.60 (m, 1H), 4.00-4.15 (m, 3H), 4.22 (m, 1H), 4.89 (m, 1H), 6.05 (s, 2H), 6.92 (d, J=8.2 Hz, 1H), 7.18 (d, J=9.5 Hz, 1H), 7.41 (dd, J=8.1, 1.7 Hz, 1H), 7.49 (d, J=1.7 Hz, 1H), 7.81 (d, J=9.5 Hz, 1H); MS (DCI/NH₃) m/z 397 (M+H)⁺.

### EXAMPLE OF 39C

### (1R, 5R)-3-(6-Benzo[1,3]dioxol-5-yl-pyridazin-3-yl)-3,6-diaza-bicyclo[3.2.0]heptane p-toluenesulfonate

The product of Example 39B (220 mg, 0.55 mmol) was treated with TsOH.H₂O (230 mg, 1.21 mmol) in EtOAc (20 mL) at 80° C. over 2 h. The title compound was obtained as a solid (280 mg, 0.44 mmol, 79% yield). ¹H NMR (MeOH-d₄, 300 MHz) δ 2.33 (s, 6H), 3.55-3.70 (m, 2H), 3.78 (dd, J=12.1, 5.8 Hz, 1H), 3.88 (dd, J=11.9, 5.1 Hz, 1H), 4.20-4.36 (m, 2H), 4.56 (d, J=12.9 Hz, 1H), 5.21 (t, J=5.8 Hz, 1H), 6.11 (s, 2H), 7.04 (d, J=8.2 Hz, 1H), 7.20 (d, J=8.1 Hz, 4H), 7.46-7.52 (m, 2H), 7.65 (d, J=8.4 Hz, 4H), 7.88 (d, J=9.9 Hz, 1H), 8.33 (d, J=9.8 Hz, 1H); MS (DCI/NH₃) m/z 297 (M+H)⁺; Anal. calculated for C₁₆H₁₆N₄O₂.2C₇H₈SO₃: C, 56.24; H, 5.03; N, 8.74. Found: C, 56.01; H, 4.94; N, 8.51.

### EXAMPLE 40

### (1R, 5R)-3-(6-Benzo[1,3]dioxol-5-yl-pyridazin-3-yl)-6-methyl-3,6-diaza-bicyclo[3.2.0]heptane

### EXAMPLE 40A

### (1R, 5R)-3-(6-Benzo[1,3]dioxol-5-yl-pyridazin-3-yl)-6-methyl-3,6-diaza-bicyclo[3.2.0]heptane

The product of Example 39B (200 mg, 0.31 mmol) was treated with formalin following the procedure of Example 34A. The title compound was obtained as a solid (90 mg, 0.28 mmol, 93% yield). ¹H NMR (MeOH-d₄, 300 MHz) δ 2.49 (s, 3H), 3.28 (m, 1H), 3.39-3.57 (m, 3H), 4.00 (dd, J=11.2, 2.1 Hz, 1H), 4.05 (d, J=12.5 Hz, 1H), 4.19 (dd, J=6.7, 4.4 Hz, 1H), 4.82 (m, 1H), 6.01(s, 2H), 6.93 (d, J=8.1 Hz, 1H), 7.15 (d, J=9.5 Hz, 4H), 7.3 (dd, J=8.2, 1.7 Hz 1H), 7.48 (d, J=1.3 Hz, 1H), 7.83 (d, J=9.4 Hz, 1H); MS (DCI/NH₃) m/z 311 (M+H)⁺.

### EXAMPLE 40B

### (1R, 5R)-3-(6-Benzo[1,3]dioxol-5-yl-pyridazin-3-yl)-6-methyl-3,6-diaza-bicyclo[3.2.0]heptane p-toluenesulfonate

The product of Example 40A (90 mg, 0.28 mmol) was treated with TsOH.H₂O (76 mg, 0.4 mmol) in EtOAc (10 mL) at ambient temperature for ˜18 h. The title compound was obtained as a solid (140 mg, 0.21 mmol, 54% yield). ¹H NMR (MeOH-d₄, 300 MHz) δ 2.31 (s, 6H), 3.04 (s, 3H), 3.60-3.72 (m, 2H), 3.72 (dd, J=13.9, 5.4 Hz, 1H), 4.19-4.28 (m, 3H), 4.61 (d, J=13.9 Hz, 1H), 5.07 (m, 1H), 6.10 (s, 2H), 7.04 (d, J=7.8 Hz, 1H), 7.18 (d, J=7.8 Hz, 4H), 7.48 (m, 2H), 7.64 (d, J=8.1 Hz, 4H), 7.75 (d, J=9.5 Hz, 1H), 8.22 (d, J=9.8 Hz, 1H); MS (DCI/NH₃) m/z 311 (M+H)⁺; Anal. calculated for C₁₇H₁₈N₄O₂.2C₇H₈SO₃: C, 56.87; H, 5.23; N, 8.56. Found: C, 56.85; H, 5.37; N, 8.74.

### EXAMPLE 41

### (1R, 5R)-1-{4-[5-(3,6-Diaza-bicyclo[3.2.0]hept-3-yl)-pyridin-2-yl-phenyl}-ethanone

### EXAMPLE 41A

### (1S, 5R)-3-(6-Chloro-pyridin-3-yl)-3,6-diaza-bicyclo[3.2.0]heptane-6-carboxylic Acid Tert-Butyl Ester

The product of Example 37G (2.0 g, 10 mmol), was coupled with 5-Bromo-2-chloro-pyridine (Aldrich, 2.3 g, 12 mmol) catalyzed by Pd₂(dba)₃ (Strem, 90 mg, 0.1 mmol) and racemic-2,2′-bis(diphenylphosphino)-1,1′-binaphthyl (BINAP, Strem, 200 mg, 0.3 mmol) with t-BuONa (1.0 g, 10 mmol) in toluene (30 mL) at 110° C. under N₂ for ˜18 h. After the reaction went to completion, the mixture was cooled to ambient temperature and quenched with 50 mL of water. The layers were separated and the aqueous layer was extracted with CHCl₃ (3×25 mL). The extracts were combined and concentrated. The residue was purified by chromatography (SiO₂, 90:10:2 CH₂Cl₂: MeOH:NH₄OH, R_{f}. 0.20) and the title compound was obtained as an oil (2.0 g, 6.4 mmol, 65% yield). ¹H NMR (MeOH-d₄, 300 MHz) δ 1.44 (s, 9H), 2.84-2.94 (m, 1H), 2.98 (dd, J=6.4 Hz, J=10.5 Hz, 1H), 3.16-3.30 (m, 1H), 3.51-3.67 (m, 1H), 3.76 (d, J=9.8 Hz, 1H), 3.84-3.99 (m, 1H), 4.02-4.15 (m, 1H), 7.24-7.29 (m, 2H), 7.85 (s, 1H); MS (DCI/NH₃) m/z 310 (M+H)⁺, 312 (M+H)⁺.

### EXAMPLE 41B

### (1S, 5R)-3-[6-(4-Acetyl-phenyl)-pyridin-3-yl]-3,6-diaza-bicyclo[3.2.0]heptane-6-carboxylic Acid Tert-Butyl Ester

The product of Example 41A (200 mg, 0.65 mmol) was coupled with 4-acetylphenylboronic acid (Aldrich, 213 mg, 1.3 mmol) following the procedure of Example 39B. The title compound was obtained as an oil (170 mg, 0.43 mmol, 67% yield). ¹H NMR (MeOH-d₄, 300 MHz) δ 1.46 (s, 9H), 2.63 (s, 3H), 2.94-3.04 (m, 1H), 3.09 (dd, J=6.8 Hz, J=10.5 Hz, 1H), 3.56-3.70 (m, 1H), 3.88 (d, J=10.5 Hz, 1H), 3.96-4.19 (m, 2H), 7.33 (dd, J=2.7 Hz, J=8.8 Hz, 1H), 7.82 (d, J=8.5 Hz, 1H), 7.99-8.13 (m, 5H), 8.21 (d, J=2.7 Hz, 1H); MS (DCI/NH₃) m/z 394 (M+H)⁺.

### EXAMPLE 41C

### (1R, 5R)-1-{4-[5-(3,6-Diaza-bicyclo[3.2.0]hept-3-yl)-pyridin-2-yl]-phenyl}-ethanone p-toluenesulfonate

The product of Example 41B (170 mg, 0.43 mmol) was treated with p-TsOH.H₂O (163 mg, 0.86 mmol) in EtOAc (10 mL) at 80° C. over 10 hours. The title was obtained as a solid (191 mg, 0.30 mmol, 66% yield). ¹H NMR (MeOH-d₄, 300 MHz) δ 2.35 (s, 6H), 2.67 (s, 3H), 3.43 (dd, J=5.4 Hz, J=12.9 Hz, 1H), 3.55-3.67 (m, 1H), 4.09 (d, J=11.2 Hz, 1H), 4.26-4.37 (m, 2H), 5.14 (t, J=6.5 Hz, 1H), 7.21 (d, J=7.8 Hz, 4H), 7.68 (d, J=8.1 Hz, 4H), 7.95-8.01 (m, 3H), 8.17-8.22 (m, 3H), 8.28 (d, J=3.1 Hz, 1H); MS (DCI/NH₃) m/z 294 (M+H)⁺; Anal. calculated for C₁₈H₁₉N₃O₀₂C₇H₈O₃S: C, 60.26; H, 5.53; N, 6.59. Found: C, 59.98; H, 5.35, N, 6.51.

### EXAMPLE 42

### (1R, 5R)-1-{4-[5-(6-Methyl-3,6-diaza-bicyclo[3.2.0]hept-3-yl)-pyridin-2-yl]-phenyl}-ethanone

### EXAMPLE 42A

### (1R, 5R)-1-[4-[5-(6-Methyl-3,6-diaza-bicyclo[3.2.0]hept-3-yl)-pyridin-2-yl]-phenyl]-ethanone

The product of Example 41C (150 mg, 0.24 mmol) was treated with formalin following the procedure of Example 34A. The title compound was obtained as a yellow oil (74 mg, 0.24 mmol, 100% yield). ¹H NMR (MeOH-d₄, 300 MHz) δ 2.40 (s, 3H), 2.63 (s, 3H), 3.07 (dd, J=4.4 Hz, J=10.9 Hz, 1H), 3.20-3.40 (m, 4H), 3.80 (m, 2H), 4.03 (dd, J=4.4 Hz, J=6.5 Hz, 1H), 7.25 (dd, J=3.1 Hz, J=8.8 Hz, 1H), 7.82 (d, J=8.8 Hz, 1H), 7.98-8.10 (m, 4H), 8.15 (d, J=3.1 Hz, 1H); MS (DCI/NH₃) m/z 308 (M+H)⁺.

### EXAMPLE 42B

### (1R, 5R)-1-4-[5-(6-Methyl-3,6-diaza-bicyclo[3.2.0]hept-3-yl)-pyridin-2-yl]-phenyl-ethanone p-toluenesulfonate

The product of Example 42A (74 mg, 0.24 mmol) was treated with p-TsOH.H₂O (95 mg, 0.5 mmol) in EtOAc (10 mL) at ambient temperature over 16 hours. The title compound was obtained as a white solid (157 mg, 0.24 mmol, 100% yield). ¹H NMR (MeOH-d₄, 300 MHz) δ 2.33 (s, 6H), 2.67 (s, 3H), 3.04 (s, 3H), 3.33-3.50 (m, 2H), 3.57-3.70 (m, 1H), 4.05-4.22 (m, 3H), 4.41 (d, J=12.9 Hz, 1H), 5.02 (t, J=4.7 Hz, 1H), 7.21 (d, J=8.1 Hz, 4H), 7.67 (d, J=8.1 Hz, 4H), 7.92-8.05 (m, 3H), 8.15-8.30 (m, 4H); MS (DCI/NH₃) m/z 308 (M+H)⁺; Anal. calculated for C₁₉H₂₁N₃O.2C₇H₈O₃S: C, 60.81; H, 5.72; N, 6.45. Found: C, 60.51; H, 5.73; N 6.24.

### EXAMPLE 43

### 6a-Methyl-5-(6-m-tolyl-pyridin-3-yl)-octahydro-pyrrolo[3,4-b]pyrrole

### EXAMPLE 43A

### Allyl-(2-hydroxy-propyl)-carbamic Acid Benzyl Ester

The product of Example 33C (13.2 g, 56.6 mmol) in THF (100 mL) was treated with MeMgBr (3M in THF, 24.5 mL, 73.5 mmol) at −78° C. over 2 hours. The mixture was then warmed to ambient temperature. The reaction was quenched with saturated, aqueous NH₄Cl solution (50 mL) at 0° C., the layers were separated and the aqueous layer was extracted with EtOAc (3×200 mL). The organic layers were combined and concentrated under reduced pressure. The residues were purified by column chromatography (SiO₂, 40% hexanes-ethyl acetate) to give the title compound (6.48 g, 26 mmol, 46% yield). ¹H NMR (CDC₃, 300 MHz) δ 1.16 (d, J=6.4 Hz, 3H), 3.14-3.41 (m, 2H), 3.83-4.09 (m, 3H), 5.02-5.22 (m, 4H), 5.69-5.90 (m, 1H), 7.20-7.40 (m, 5H); MS (DCI/NH₃) m/z 250 (M+H)⁺, 267 (M+NH₄)⁺.

### EXAMPLE 43B

### Allyl-(2-oxo-propyl)-carbamic Acid Benzyl Ester

Dimethylsulfoxide (DMSO, 4.7 g, 60.1 mmol) was added slowly into a solution of oxalyl chloride (3.82 g, 30.1 mmol) in CH₂Cl₂ (150 mL) at −78° C. After the addition was complete, the mixture was stirred for 15 minutes. The product of Example 43A (6.25 g, 25.1 mmol) in CH₂Cl₂ (20 mL) was added to the above mixture at −78° C. After the mixture was stirred for 30 minutes, triethylamine (12.6 g, 125 mmol) was added. The reaction mixture was then warmed slowly to ambient temperature. After the reaction was complete, it was quenched with water (10 mL). The layers were separated and the aqueous layer was extracted with EtOAc (3×200 ML). The extracts were combined and concentrated under reduced pressure. The residue was purified by column chromatography (SiO₂, 40% hexanes-ethyl acetate) to give the title compound (4.3 g, 17.4 mmol, 70% yield). ¹H NMR (CDCl₃, 300 MHz) δ 2.05 (s, 1.4H), 2.14 (s, 1.6H), 3.91-4.08 (m, 4H), 5.06-5.21 (m, 4H), 5.68-5.86 (m, 1H), 7.25-7.40 (m, 5H); MS (DCI/NH₃) m/z 248 (M+H)⁺, 265 (M+NH₄)⁺.

### EXAMPLE 43C

### 1-Benzyl-6a-methyl-hexahydro-pyrrolo[3,4-b]pyrrole-5-carboxylic Acid Benzyl Ester

The product of Example 43B (3.0 g, 12.1 mmol) was treated with benzylaminoacetic acid (Aldrich, 2.0 g, 12.1 mmol) in toluene (50 mL) at 110° C. over 2 days. The toluene was removed under reduced pressure and the residue was purified by column chromatography (SiO₂, 40% hexanes-ethyl acetate) to give the title compound (2.8 g, 8.0 mmol, 66% yield). ¹H NMR (MeOH-d₄, 300 MHz) δ 1.23 (s, 3H), 1.49-1.64 (m, 1H), 1.93-2.10 (m, 1H), 2.36-2.51 (m, 1H), 2.56-2.67 (m, 1H), 2.73-2.87 (m, 1H), 3.10 (d, J=11.5 Hz, 1H), 3.32-3.41 (m, 1H), 3.52 (d, J=13.2 Hz, 1H), 3.58-3.78 (m, 3H), 5.03-5.22 (m, 2H), 7.14-7.42 (m, 10H); MS (DCI/NH₃) m/z 351 (M+H)⁺.

### EXAMPLE 43D

### 1-Benzyl-6a-methyl-octahydro-pyrrolo[3,4-b]pyrrole

The product of Example 43C (1.7 g, 4.85 mmol) was treated with Pd/C (10 wt %, 300 mg) i-PrOH (50 mL) at ambient temperature under 1 atm of H₂ for 18 h. After the reaction went completion, the catalyst was filtered off and the filtrate was concentrated under reduced pressure to give the title compound (0.7 g, 3.2 mmol, 66% yield). ¹H NMR (MeOH-d₄, 300 MHz) δ 1.23 (s, 3H), 1.32-1.46 (m, 1H), 1.94-2.07 (m, 1H), 2.23-2.39 (m, 2H), 2.46-2.56 (m, 1H), 2.66-2.75 (m, 2H), 2.95-3.04 (m, 2H), 3.62 (d, J=12.9 Hz, 1H), 3.73 (d, J=12.9 Hz, 1H), 7.13-7.37 (m, 5H); MS (DCI/NH₃) m/z 217 (M+H)⁺.

### EXAMPLE 43E

### 1-Benzyl-6a-methyl-hexahydro-pyrrolo[3,4-b]pyrrole-5-carboxylic Acid Tert-Butyl Ester

The product of Example 43D (700 mg, 3.24 mmol) was treated with di-tert-butyl dicarbonate (706 mg, 3.24 mmol) and Et₃N (2 mL) in CH₂Cl₂ (10 mL) for 16 hours. The mixture was then concentrated under reduced pressure and purified by column chromatography (SiO₂, 40% hexanes-ethyl acetate) to give the title compound (1.02 g, 3.24 mmol, 100% yield). ¹H NMR (MeOH-d₄, 300 MHz) δ 1.22 (s, 3H), 1.47 (s, 9H), 1.49-1.62 (m, 1H), 1.94-2.11 (m, 1H), 2.34-2.46 (m, 1H), 2.57-2.68 (m, 1H), 2.73-2.87 (m, 1H), 3.02 (d, J=111.5 Hz, 1H), 3.21-3.27 (m, 1H), 3.50-3.74 (m, 4H), 7.15-7.32 (m, 5H); MS (DCI/NH₃) m/z 317 (M+H)⁺.

### EXAMPLE 43F

### 6a-Methyl-hexahydro-pyrrolo[3,4-b]pyrrole-1,5-dicarboxylic Acid 1-benzyl Ester 5-Tert-Butyl Ester

The product of Example 43E (1.02 g, 3.24 mmol) was treated with Pd/C (10 wt %, 100 mg) in MeOH (50 mL) under 1 atm. H₂ at 50° C. for 16 hours. The reaction mixture was cooled to ambient temperature. The catalyst was filtered off and the filtrate was concentrated under reduced pressure. The residue was treated with CbzCl (0.5 mL, 3.5 mmol) and Et₃N (3 mL) in CH₂Cl₂ (20 mL) at 0° C. for 2 h. After the reaction was complete, it was quenched with water (5 mL) and extracted with CH₂Cl₂ (3×20 mL). The extracts were combined and concentrated under reduced pressure. The residue was purified by column chromatography (SiO₂, 40% hexanes-ethyl acetate) to give the title compound (0.87 g, 2.42 mmol, 75% yield). ¹H NMR (MeOH-d₄, 300 MHz) δ 1.36-1.50 (m, 13H), 1.67-1.80 (m, 1H), 1.98-2.14 (m, 1H), 2.53-2.68 (m, 1H), 3.14-3.32 (m, 2H), 3.49-3.68 (m, 3H), 5.09 (s, 2H), 7.22-7.42 (m, 5H).

### EXAMPLE 43G

### 6a-Methyl-hexahydro-pyrrolo[3,4-b]pyrrole-1-carboxylic Acid Benzyl Ester

The product of Example 43F (0.8 g, 2.22 mmol) was treated with TFA (5 mL) in CH₂Cl₂ (10 mL) at ambient temperature for 1 h. The mixture was then concentrated under reduced pressure and the residue was purified by column chromatography (SiO₂, 90:9:1 CH₂Cl₂: MeOH:NH₄OH) to give the title compound (0.32 g, 1.23 mmol, 55% yield). ¹H NMR (MeOH-d₄, 300 MHz) δ 1.42, 1.47 (s, 3H, rotamers), 1.63-1.75 (m, 1H), 1.98-2.13 (m, 1H), 2.37-2.52 (m, 1H), 2.62-2.76 (m, 2H), 3.00-3.12 (m, 1H), 3.26, 3.47 (d, J=12.6 Hz, 1H, rotamers), 3.53-3.62 (m, 2H), 5.08, 5.13 (s, 2H, rotamers), 7.25-7.42 (m, 5H); MS (DCI/NH₃) m/z 261 (M+H)⁺.

### EXAMPLE 43H

### 5-(6-Chloro-pyridin-3-yl)-6a-methyl-hexahydro-pyrrolo[3,4-b]pyrrole-1-carboxylic Acid Benzyl Ester

The product of Example 43G (200 mg, 0.77 mmol) was coupled with 5-bromo-2-chloro-pyridine (Aldrich, 210 mg, 1.09 mmol) following the procedure of Example 41A. The title compound was obtained as an oil (199 mg, 0.53 mmol, yield 70%). ¹H NMR (MeOH-d₄, 300 MHz) δ 1.52, 1.56 (s, 2H, rotamers), 1.76-1.91 (m, 1H), 2.05-2.24 (m, 1H), 2.71-2.84 (m, 1H), 3.16-3.27 (m, 2H), 3.46-3.68 (m, 3H), 3.78, 4.07 (d, J=10.2 Hz, 1H, rotamers), 5.06-5.30 (m, 2H), 6.87-7.66 (m, 8H); MS (DCI/NH₃) m/z 374 (M+H)⁺, 372 (M+H)⁺.

### EXAMPLE 431

### 6a-Methyl-5-(6-m-tolyl-pyridin-3-yl)-hexahydro-pyrrolo[3,4-b]pyrrole-1-carboxylic Acid Benzyl Ester

The product of Example 43H (186 mg, 0.50 mmol) was coupled with m-tolylboronic acid (100 mg, 0.74 mmol) following the procedure of Example 39B to give 120 mg of the title compound (0.28 mmol, 56% yield). ¹H NMR (MeOH-d₄, 300 MHz) δ 1.55, 1.59 (s, 3H, rotamers), 1.88 (m, 1H), 2.16 (m, 1H), 2.40 (s, 3H), 2.80 (m, 1H), 3.51-3.73 (m, 3H), 3.88 (d, J=10.5 Hz, 0.35H), 4.15 (d, J=10.5 Hz, 0.65H), 5.08-5.31 (m, 2H), 6.94-7.18 (m, 2H), 7.23-7.48 (m, 6H), 7.54-7.68 (m, 3H), 7.80-7.98 (m, 1H); MS (DCI/NH₃) m/z 428 (M+H)⁺.

### EXAMPLE 43J

### 6a-Methyl-5-(6-m-tolyl-pyridin-3-yl)-octahydro-pyrrolo[3,4-b]pyrrole Fumarate

The product of Example 431 (120 mg, 0.28 mmol) was treated with Pd/C (10 wt %, 50 mg) in MeOH (10 mL) under 1 atm. H₂ at ambient temperature for 18 h. The catalyst was removed by filtration and the filtrate was concentrated under reduced pressure. The residue (52 mg, 1.8 mmol, 64% yield free base) was treated with fumaric acid (25 mg, 0.21 mmol) in EtOAc/MeOH (10:1) (10 mL) at ambient temperature for ˜18 h. The title product was obtained as a solid (58 mg, 1.4 mmol, 77% yield). ¹H NMR (MeOH-d₄, 300 MHz) δ 1.66 (s, 3H), 2.06-2.13 (m, 1H), 2.40 (s, 3H), 2.44-2.54 (m, 1H), 2.84-2.93 (m, 1H), 3.35-3.62 (m, 4H), 4.00 (d, J=11.2 Hz, 1H), 6.69 (s, 2.2H), 7.19-7.34 (m, 3H), 7.59-7.72 (m, 3H), 8.09 (d, J=2.4 Hz, 1H); MS (DCI/NH₃) m/z 294 (M+H)⁺; Anal. calculated for C₁₉H₂₃N₃.1.1C₄H₄O₄, C, 66.74; H, 6.56; N, 9.98. Found: C, 66.77; H, 6.59; N, 9.76.

### EXAMPLE 44

### 2-(5-Phenyl-thiazol-2-yl)-octahydro-pyrrolo[3,4-c]pyrrole

### EXAMPLE 44A

### 5-(5-Bromo-thiazol-2-yl)-hexahydro-pyrrolo[3,4-c]pyrrole-2-carboxylic Acid Tert-Butyl Ester

To the product of Example 6C (1.0 g, 4.2 mmol) in N,N-diisopropylethylamine (1.5 mL, 8.4 mmol) was added 2,5-dibromothiazole (Aldrich, 0.89 g, 4.2 mmol). This mixture was warmed to 110° C. and stirred for 2 h. The reaction mixture was cooled to ambient temperature, concentrated under reduced pressure, and purified by column chromatography (SiO₂, 20-40% ethyl acetate-hexanes gradient) to afford 1.1 g of the title compound (2.9 mmol, 69% yield). ¹H NMR (CDCl₃, 300 MHz) δ 1.46 (s, 9H), 2.97-3.09 (m, 2H), 3.21-3.41 (m, 4H), 3.60-3.71 (m, 4H), 7.09 (s, 1H); MS (DCI/NH₃) m/z 376 (M+H)⁺.

### EXAMPLE 44B

### 5-(5-Phenyl-thiazol-2-yl)-hexahydro-pyrrolo[3,4-c]pyrrole-2-carboxylic Acid Tert-Butyl Ester

To the product of Example 44A (0.55 g, 1.46 mmol) in 10 mL dioxane was added phenylboronic acid (0.196 g, 1.53 mmol), t-Bu₃P (Strem, 0.080 g, 0.15 mmol), and Cs₂CO₃ (0.95 g, 2.9 mmol). The mixture was warmed to 80° C. and stirred for 12 hours. The reaction mixture was cooled to ambient temperature, concentrated under reduced pressure, and purified by column chromatography (SiO₂, 50% hexanes/ethyl acetate) to afford 0.28 g of the title compound (0.76 mmol, 52% yield). ¹H NMR (CDCl₃, 300 MHz) δ 1.46 (s, 9H), 3.00-3.17 (m, 2H), 3.24-3.54 (m, 4H), 3.61-3.72 (m, 2H), 3.74-3.86 (m, 2H), 7.22 (m, 1H), 7.34 (t, J=7.6 Hz, 2H), 7.42(d, J=8.5 Hz, 3H); MS (DCI/NH₃) m/z 372 (M+H)⁺.

### EXAMPLE 44C

### 2-(5-Phenyl-thiazol-2-yl)-octahydro-pyrrolo[3,4-c]pyrrole Trifluoroacetate

The product of Example 44B (0.28 g, 0.75 mmol) in 3 mL of CH₂Cl₂ was treated with 3 mL of trifluoroacetic acid. The solution was stirred for 12 hours and then concentrated under reduced pressure. The residue was triturated with ethyl acetate and diethyl ether and then dried under reduced pressure to afford 0.23 g of title compound (0.59 mmol, 79% yield). ¹H NMR (MeOH-d₄, 300 MHz) δ 2.36 (s, 3H), 2.95 (s, 3H) 3.33-3.49 (m, 4H), 3.58-3.71 (m, 4H), 3.83-3.93 (m, 2H), 7.31-7.46 (m, 3H), 7.51-7.57 (m, 2H), 7.68 (s, 1H); MS (DCI/NH₃) m/z 272 (M+H)⁺.

### EXAMPLE 44D

### 2-(5-Phenyl-thiazol-2-yl)-octahydro-pyrrolo[3,4-c]pyrrole p-toluenesulfonate

The product in Example 44C (0.046 g, 0.17 mmol) was treated with 2 mL of saturated aqueous potassium carbonate. The solution stirred for 4 hours and was then diluted with methylene chloride. The layers were separated, and the aqueous phase was extracted twice with methylene chloride. The combined organic extracts were dried over potassium carbonate, filtered and concentrated under reduced pressure. This residue was processed as described in Example 45B to provide 0.011 g of title compound (0.025 mmol, 15% yield). ¹H NMR (MeOH-d₄, 300 MHz) δ 3.57-3.67 (m, 3H), 3.74-3.84 (m, 2H), 7.22 (d, 2H, J=7.8 Hz), 7.29 (m, 1H), 7.35-7.42 (m, 2H), 7.47-7.52 (m, 2H), 7.56 (s, 1H), 7.70 (m, 2H); MS (DCI/NH₃) m/z 272 (M+H)⁺.

### EXAMPLE 45

### 2-Methyl-5-(5-phenyl-thiazol-2-yl)-octahydro-pyrrolo[3,4-c]pyrrole

### EXAMPLE 45A

### 2-Methyl-5-(5-phenyl-thiazol-2-yl)-octahydro-pyrrolo[3,4-c]pyrrole

To the product of Example 44C (0.18 g, 0.47 mmol) in 2 mL of aqueous formaldehyde (37%) at 0° C. was added sodium triacetoxyborohydride (0.20 g, 0.94 mmol). The reaction mixture was stirred at ambient temperature for 6 h. The mixture was then diluted with ethyl acetate and washed with saturated, aqueous NaHCO₃ (2×5 mL) and saturated, aqueous NaCl (1×5 mL). The organic extract was dried over MgSO₄, filtered, and concentrated under reduced pressure. Purification by column chromatography (SiO₂, 0-20% methanol-CH₂Cl₂ gradient) afforded 0.035 g of the title compound (0.12 mmol, 25% yield). ¹H NMR (CDCl₃, 400 MHz) δ 2.38 (s, 3H), 2.52 (d, J=8.9 Hz, 2H), 2.79 (s, 2H), 3.06 (s, 2H), 3.42 (d, J=10.1 Hz, 2H), 3.67 (m, 2H), 7.19 (t, J=7.4 Hz, 1H), 7.32 (t, J=6.8 Hz, 2H), 7.41-7.42 (m, 3H); MS (DCI/NH₃) m/z 286 (M+H)⁺.

### EXAMPLE 45B

### 2-Methyl-5-(5-phenyl-thiazol-2-yl)-octahydro-pyrrolo[3,4-c]pyrrole p-toluenesulfonate

To the product of Example 45A (0.035 g, 0.12 mmol) in 2 mL of ethyl acetate was added p-toluenesulfonic acid monohydrate (0.023 g, 0.12 mmol). The solution stirred for 12 hours and then was concentrated under reduced pressure. The residue was triturated with ethyl acetate and diethyl ether and then dried under reduced pressure to give 0.12 g of the title compound (0.025 mmol, 17% yield). ¹H NMR (MeOH-d₄, 300 MHz) δ 2.36 (s, 3H), 2.95 (s, 3H), 3.33-3.43 (m, 2H), 3.57-3.65 (m, 4H), 7.24 (t, J=8.0 Hz, 3H), 7.35 (t, J=7.6 Hz, 2H), 7.45-7.50 (m, 3H), 7.7 (d, J=8.1 Hz, 2H); MS (DCI/NH₃) m/z 286 (M+H)⁺. Anal. calculated for C₁₆H₁₉N₃S.C₇H₈O₃S: C, 60.37; H, 5.95; N, 9.18. Found: C, 60.04; H, 6.04; N, 9.15.

### EXAMPLE 46

### Determination of Biological Activity

To determine the effectiveness of representative compounds of this invention as α7 nAChRs, the compounds of the invention were evaluated according to the [3H]-methyllycaconitine (MLA) binding assay and considering the [3H]-cytisine binding assay, which were performed as described below.

[3H]-Cytisine Binding

Binding conditions were modified from the procedures described in Pabreza L A, Dhawan, S, Kellar K J, [³H]-Cytisine Binding to Nicotinic Cholinergic Receptors in Brain, Mol. Pharm. 39: 9-12, 1991. Membrane enriched fractions from rat brain minus cerebellum (ABS Inc., Wilmington, Del.) were slowly thawed at 4° C., washed and resuspended in 30 volumes of BSS-Tris buffer (120 mM NaCl/5 mM KCl/2 mM CaCl₂/2 mM MgCl₂/50 mM Tris-Cl, pH 7.4, 4° C.). Samples containing 100-200 μg of protein and 0.75 nM [3H]-cytisine (30 Cᵢ/mmol; Perkin Elmer/NEN Life Science Products, Boston, Mass.) were incubated in a final volume of 500 μL for 75 minutes at 4° C. Seven log-dilution concentrations of each compound were tested in duplicate. Non-specific binding was determined in the presence of 10 μM (−)-nicotine. Bound radioactivity was isolated by vacuum filtration onto prewetted glass fiber filter plates (Millipore, Bedford, Mass.) using a 96-well filtration apparatus (Packard Instruments, Meriden, Conn.) and were then rapidly rinsed with 2 mL of ice-cold BSS buffer (120 mM NaCl/5 mM KCl/2 mM CaCl₂/2 mM MgCl₂). Packard MicroScint-20® scintillation cocktail (40 μL) was added to each well and radioactivity determined using a Packard TopCount® instrument. The IC₅₀ values were determined by nonlinear regression in Microsoft Excel® software. Kᵢ values were calculated from the IC₅₀s using the Cheng-Prusoff equation, where Kᵢ=IC₅₀/1+[Ligand]/K_{D}].

[3H1-Methyllycaconitine (MLA) Binding

Binding conditions were similar to those for [3H]-cytisine binding. Membrane enriched fractions from rat brain minus cerebellum (ABS Inc., Wilmington, Del.) were slowly thawed at 4° C., washed and resuspended in 30 volumes of BSS-Tris buffer (120 mM NaCl, 5 mM KCl, 2 mM CaCl₂, 2 mM MgCl₂, and 50 mM Tris-Cl, pH 7.4, 22° C.). Samples containing 100-200 μg of protein, 5 nM [3H]-MLA (25 Cᵢ/mmol; Perkin Elmer/NEN Life Science Products, Boston, Mass.) and 0.1% bovine serum albumin (BSA, Millipore, Bedford, Mass.) were incubated in a final volume of 500 μL for 60 minutes at 22° C. Seven log-dilution concentrations of each compound were tested in duplicate. Non-specific binding was determined in the presence of 10 μM MLA. Bound radioactivity was isolated by vacuum filtration onto glass fiber filter plates prewetted with 2% BSA using a 96-well filtration apparatus (Packard Instruments, Meriden, Conn.) and were then rapidly rinsed with 2 mL of ice-cold BSS. Packard MicroScint-20° scintillation cocktail (40 μL) was added to each well and radioactivity was determined using a Packard TopCount® instrument. The IC₅₀ values were determined by nonlinear regression in Microsoft Excel® software. Kᵢ values were calculated from the IC₅₀s using the Cheng-Prusoff equation, where Kᵢ=IC₅₀/1+[Ligand]/K_{D}].

Compounds of the invention had Kᵢ values of from about 1 nanomolar to about 10 micromolar when tested by the MLA assay, many having a Kᵢ of less than 1 micromolar. [3H]-Cytisine binding values of compounds of the invention ranged from about 50 nanomolar to at least 100 micromolar. The determination of preferred compounds typically considered the Kᵢ value as measured by MLA assay in view of the Kᵢ value as measured by [3H]-cytisine binding, such that in the formula D=K_{i MLA}/K_{i 3H-cytisine}, D is about 50. Preferred compounds typically exhibited greater potency at α7 receptors compared to α4β2 receptors.

Compounds of the invention are α7 nAChRs ligands that modulate function of α7 nAChRs by altering the activity of the receptor. The compounds can be inverse agonists that inhibit the basal activity of the receptor or antagonists that completely block the action of receptor-activating agonists. The compounds also can be partial agonists that partially block or partially activate the α7 nAChR receptor or agonists that activate the receptor.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

1. A compound of the formula (I):
Z-Ar₁—Ar₂ (I)
or a pharmaceutically acceptable salt, ester, amide, or prodrug thereof, wherein:
Z is a diazabicyclic amine of the formula: 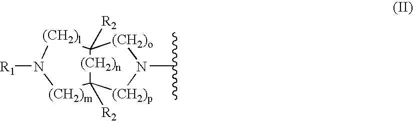
Ar₁ is a 5- or 6-membered aromatic ring of the formula: 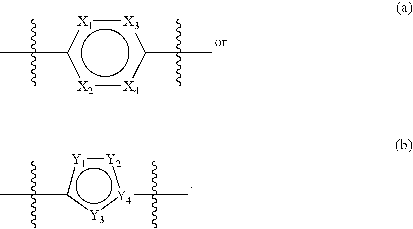
Ar₂ is selected from the group consisting of an unsubstituted or substituted 5- or 6-membered heteroaryl ring; unsubstituted or substituted bicyclic heteroaryl ring; 3,4-(methylenedioxy)phenyl; and phenyl substituted with 0, 1, 2, or 3 substituents in the meta- or para-positions; provided that when Y₁ is O or S, Y₂ is N, Y₃ is —CR₃ and R₃ is hydrogen, and Y₄ is C, then Ar₂ is not 5-tetrazolyl;
X₁, X₂, X₃, and X₄ are each independently selected from the group consisting of N and —CR₃, provided that R₃ is not hydrogen at least in one occurrence when X₁, X₂, X₃, and X₄ are all —CR₃;
Y₁, Y₂, and Y₃ are each independently selected from the group consisting of N, O, S, and —CR₃;
Y₄ is C or N, provided that when Y₄ is C at least one of Y₁, Y₂, and Y₃, is other than —CR₃;
l, m, n, o, and p are each independently selected from 0, 1, or 2, provided that the sum total of l, m, n, o, and p is 3, 4, or 5;
R₁ is independently selected from the group consisting of hydrogen, alkyl, and alkoxycarbonyl;
R₂ at each occurrence is independently selected from the group consisting of hydrogen and alkyl; and
R₃ at each occurrence is independently selected from the group consisting of hydrogen and alkyl.

2. The compound of claim 1, wherein Z is selected from the group consisting of:

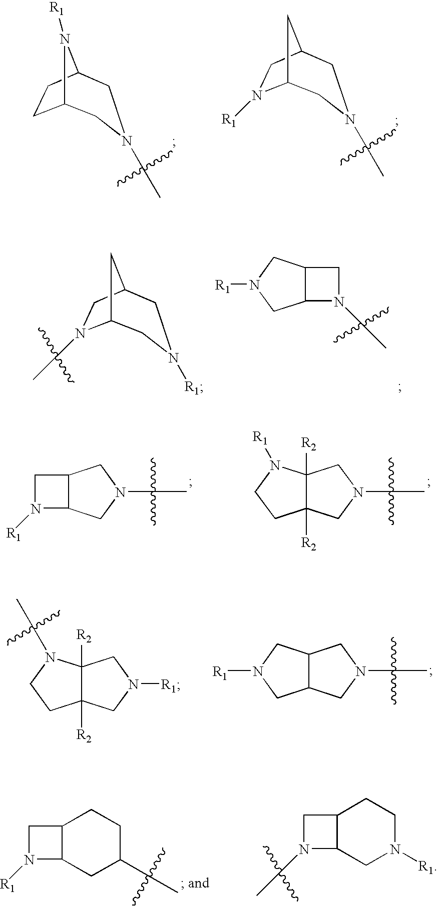

3. The compound of claim 1, wherein Ar₁ is selected from the group consisting of isoxazolyl, oxadiazolyl, pyrazolyl, pyridazinyl, pyridinyl, pyrimidinyl, thiadiazolyl, thiazolyl, thienyl, and phenyl substituted with 0 or 1 alkoxy substitutent.

4. The compound of claim 1, wherein Ar₁ is selected from the group consisting of:

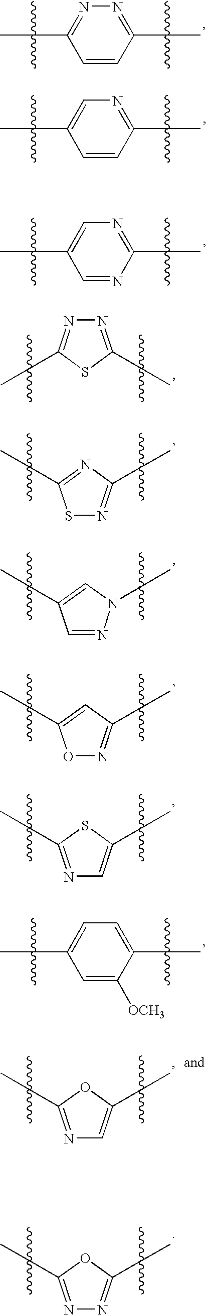

5. The compound of claim 1, wherein Ar₂ is selected from the group consisting of furyl; thienyl; pyridyl; benzothiophenyl; 3,4-(methylenedioxy)phenyl; and phenyl substituted with 0, 1, or 2 substituents selected from the group consisting of alkoxy, alkoxycarbonyl, alkyl, alkylcarbonyl, carboxy, halogen, haloalkyl, —NR_{A}R_{B}, (NR_{A}R_{B})alkyl, (NR_{A}R_{B})alkoxy, and phenyl.

6. The compound of claim 1, wherein Ar₂ is selected from the group consisting of: 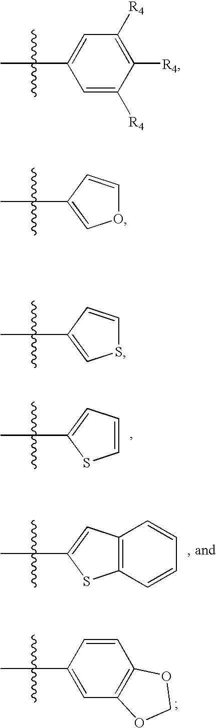
wherein R₄ at each occurrence is independently selected from the group consisting of hydrogen, alkoxy, alkoxycarbonyl, alkyl, alkylcarbonyl, carboxy, halogen, haloalkyl, —NR_{A}R_{B}, (NR_{A}R_{B})alkyl, (NR_{A}R_{B})alkoxy, and phenyl.

7. The compound of claim 6, wherein Ar₂ is selected from the group consisting of phenyl, m-methylphenyl, p-methoxyphenyl, m-trifluoromethylphenyl, and m-aminophenyl.

8. The compound of claim 1, wherein Z is 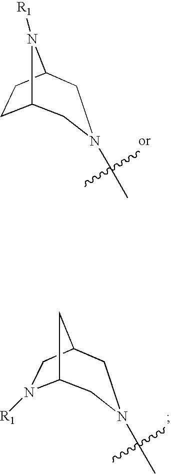
Ar₁ is pyridazinyl; and
Ar₂ is as defined in claim 1.

9. The compound of claim 8, wherein Ar₂ is phenyl or phenyl substituted with a substituent selected from the group consisting of alkyl, alkoxy, haloalkyl, —NR_{A}R_{B}, and phenyl.

10. The compound of claim 1, wherein Z is 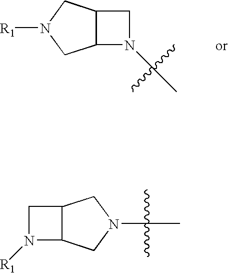
Ar₁ is pyridazinyl or pyridinyl; and
Ar₂ is as defined in claim 1.

11. The compound of claim 10, wherein Ar₂ is 3,4-(methylenedioxy)phenyl, phenyl, or phenyl substituted with 0, 1, or 2 substituents selected from the group consisting of alkyl and alkylcarbonyl.

12. The compound of claim 1, wherein Z is 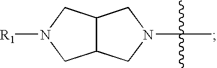
Ar₁ is pyridazinyl; and
Ar₂ is as defined in claim 1.

13. The compound of claim 12, wherein Ar₂ is phenyl or phenyl substituted with a substituent selected from the group consisting of alkyl, alkoxy, haloalkyl, —NR_{A}R_{B}, and phenyl.

14. The compound of claim 1, wherein Z is 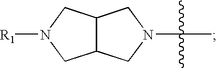
Ar₁ is pyridinyl; and
Ar₂ is defined in claim 1.

15. The compound of claim 14, wherein Ar₂ is heteroaryl or bicyclic heteroaryl, provided that Ar₂ is not 1-pyrrolyl or 1-indolyl.

16. The compound of claim 14, wherein Ar₂ is furyl, benzothiophenyl, phenyl, or phenyl substituted with a substituent selected from the group consisting of alkyl, alkoxy, haloalkyl, —NR_{A}R_{B}, and phenyl.

17. The compound of claim 1, wherein Z is 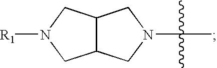
Ar₁ is either isoxazolyl, oxadiazolyl, pyrazolyl, pyrimidinyl, thiadiazolyl, or thiazolyl; and
Ar₂ is as defined in claim 1.

18. The compound of claim 17, wherein Ar₂ is phenyl or phenyl substituted with a substituent selected from the group consisting of alkyl, alkoxy, haloalkyl, —NR_{A}R_{B}, and phenyl.

19. The compound of claim 1, wherein Z is 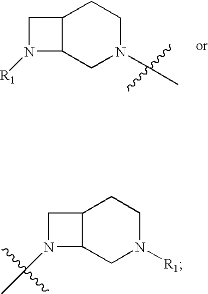
Ar₁ is pyridazinyl, pyrimidinyl, or thiazolyl; and
Ar₂ is as defined in claim 1.

20. The compound of claim 19, wherein Ar₂ is phenyl, phenyl substituted with alkylcarbonyl, or 3,4-(methylenedioxy)phenyl.

21. The compound of claim 1, wherein n is 0.

22. The compound of claim 1, or a pharmaceutically acceptable salt, ester, amide, or prodrug thereof, selected from the group consisting of:
3-(6-phenyl-pyridazin-3-yl)-3,8-diaza-bicyclo[3.2.1]octane;
8-methyl-3-(6-phenyl-pyridazin-3-yl)-3,8-diaza-bicyclo[3.2.1]octane;
6-methyl-3-(6-phenyl-pyridazin-3-yl)-3,6-diaza-bicyclo[3.2.1]octane;
3-(6-phenyl-pyridazin-3-yl)-3,8-diaza-bicyclo[4.2.0]octane;
8-methyl-3-(6-phenyl-pyridazin-3-yl)-3,8-diaza-bicyclo[4.2.0]octane;
2-(6-phenyl-pyridazin-3-yl)-octahydro-pyrrolo[3,4-c]pyrrole;
2-methyl-5-(6-phenyl-pyridazin-3-yl)-octahydro-pyrrolo[3,4-c]pyrrole;
2-(6-m-tolyl-pyridazin-3-yl)-octahydro-pyrrolo[3,4-c]pyrrole;
2-methyl-5-(6-m-tolyl-pyridazin-3-yl)-octahydro-pyrrolo[3,4-c]pyrrole;
2-[6-(4-methoxy-phenyl)-pyridazin-3-yl]-octahydro-pyrrolo[3,4-c]pyrrole;
2-(6-biphenyl-3-yl-pyridin-3-yl)-octahydro-pyrrolo[3,4-c]pyrrole;
2-(6-biphenyl-3-yl-pyridin-3-yl)-5-methyl-octahydro-pyrrolo[3,4-c]pyrrole;
2-[6-(3-trifluoromethyl-phenyl)-pyridin-3-yl]-octahydro-pyrrolo[3,4-c]pyrrole;
2-methyl-5-[6-(3-trifluoromethyl-phenyl)-pyridin-3-yl]-octahydro-pyrrolo[3,4-c]pyrrole;
3-[5-(hexahydro-pyrrolo[3,4-c]pyrrol-2-yl)-pyridin-2-yl]-phenylamine;
5-(6-furan-3-yl-pyridin-3-yl)-hexahydro-pyrrolo[3,4-c]pyrrole;
2-(6-furan-3-yl-pyridin-3-yl)-5-methyl-octahydro-pyrrolo[3,4-c]pyrrole;
2-(6-benzo[b]thiophen-2-yl-pyridin-3-yl)-octahydro-pyrrolo[3,4-c]pyrrole;
2-(6-benzo[b]thiophen-2-yl-pyridin-3-yl)-5-methyl-octahydro-pyrrolo[3,4-c]pyrrole;
2-(5-phenyl-pyridin-2-yl)-octahydro-pyrrolo[3,4-c]pyrrole;
2-methyl-5-(5-phenyl-pyridin-2-yl)-octahydro-pyrrolo[3,4-c]pyrrole;
2-(2-phenyl-pyrimidin-5-yl)-octahydro-pyrrolo[3,4-c]pyrrole;
2-methyl-5-(2-phenyl-pyrimidin-5-yl)-octahydro-pyrrolo[3,4-c]pyrrole;
diethyl-(2-{3-[6-(hexahydro-pyrrolo[3,4-c]pyrrol-2-yl)-pyridazin-3-yl]-phenoxy}-ethyl)-amine;
diethyl-(2-{3-[6-(5-methyl-hexahydro-pyrrolo[3,4-c]pyrrol-2-yl)-pyridazin-3-yl]-phenoxy}-ethyl)-amine;
2-(5-phenyl-[1,3,4]thiadiazol-2-yl)-octahydro-pyrrolo[3,4-c]pyrrole;
2-(3-phenyl-[1,2,4]thiadiazol-5-yl)-octahydro-pyrrolo[3,4-c]pyrrole;
2-methyl-5-(3-phenyl-[1,2,4]thiadiazol-5-yl)-octahydro-pyrrolo[3,4-c]pyrrole;
2-(1-phenyl-1H-pyrazol-4-yl)-octahydro-pyrrolo[3,4-c]pyrrole;
2-(2-methoxy-biphenyl-4-yl)-octahydro-pyrrolo[3,4-c]pyrrole;
2-(2-methoxy-biphenyl-4-yl)-5-methyl-octahydro-pyrrolo[3,4-c]pyrrole;
2-methyl-5-(3-phenyl-isoxazol-5-yl)-octahydro-pyrrolo[3,4-c]pyrrole;
(1S, 5S)-3-(6-phenyl-pyridazin-3-yl)-3,6-diaza-bicyclo[3.2.0]heptane;
(1S, 5S)-6-methyl-3-(6-phenyl-pyridazin-3-yl)-3,6-diaza-bicyclo[3.2.0]heptane;
(1R, 5S)-6-(6-phenyl-pyridazin-3-yl)-3,6-diaza-bicyclo[3.2.0]heptane;
(1R, 5S)-3-methyl-6-(6-phenyl-pyridazin-3-yl)-3,6-diaza-bicyclo[3.2.0]heptane;
(1R, 5R)-3-(6-phenyl-pyridazin-3-yl)-3,6-diaza-bicyclo[3.2.0]heptane;
(1R, 5R)-6-methyl-3-(6-phenyl-pyridazin-3-yl)-3,6-diaza-bicyclo[3.2.0]heptane;
(1R, 5R)-3-(6-benzo[1,3]dioxol-5-yl-pyridazin-3-yl)-3,6-diaza-bicyclo[3.2.0]heptane;
(1R, 5R)-3-(6-benzo[1,3]dioxol-5-yl-pyridazin-3-yl)-6-methyl-3,6-diaza-bicyclo[3.2.0]heptane;
(1R, 5R)-1-{4-[5-(3,6-diaza-bicyclo[3.2.0]hept-3-yl)-pyridin-2-yl]-phenyl}-ethanone;
(1R, 5R)-1-{4-[5-(6-methyl-3,6-diaza-bicyclo[3.2.0]hept-3-yl)-pyridin-2-yl]-phenyl}-ethanone;
6a-methyl-5-(6-m-tolyl-pyridin-3-yl)-octahydro-pyrrolo[3,4-b]pyrrole;
2-(5-phenyl-thiazol-2-yl)-octahydro-pyrrolo[3,4-c]pyrrole; and
2-methyl-5-(5-phenyl-thiazol-2-yl)-octahydro-pyrrolo[3,4-c]pyrrole.

23. A pharmaceutical composition comprising a therapeutically effective amount of a compound of claim 1 in combination with a pharmaceutically acceptable carrier.

24. A method of selectively modulating the effects of α7 nicotinic acetylcholine receptors in a mammal comprising administering an effective amount of a compound of claim 1.

25. A method for treating a condition or disorder modulated by an α7 nicotinic acetylcholine receptor comprising the step of administering a compound of claim 1.

26. The method according to claim 25, wherein the condition or disorder is selected from the group consisting of attention deficit disorder, attention deficit hyperactivity disorder (ADHD), Alzheimer's disease (AD), mild cognitive impairment, senile dementia, AIDS dementia, Pick's Disease, dementia associated with Lewy bodies, and dementia associated with Down's syndrome.

27. The method according to claim 25, wherein the condition or disorder is selected from the group consisting of a cognitive disorder, neurodegeneration, and schizophrenia.

28. The method according to claim 25, further comprising administering a compound of claim 1 in combination with an atypical antipsychotic.
